SEQUENCE LISTING (53 sequences)

SEQ ID NO: 1 - DNA, 80 bp, synthetic construct
  source 1..80: mol_type = other DNA
agcacacattgctggttctgctcacgtctgactacaggcattccgtctgtctcttatacacatctccgagcccacgagac

SEQ ID NO: 2 - DNA, 80 bp, synthetic construct
  source 1..80: mol_type = other DNA
agcacacattgctggttctgctcacgtcaacctagtgcgacgatacctgtctcttatacacatctccgagcccacgagac

SEQ ID NO: 3 - DNA, 80 bp, synthetic construct
  source 1..80: mol_type = other DNA
agcacacattgctggttctgctcacgccgaggtctgtcacagtgtactgtctcttatacacatctccgagcccacgagac

SEQ ID NO: 4 - DNA, 80 bp, synthetic construct
  source 1..80: mol_type = other DNA
agcacacattgctggttctgctcacggtgaaccgtatctgacggcactgtctcttatacacatctccgagcccacgagac

SEQ ID NO: 5 - DNA, 80 bp, synthetic construct
  source 1..80: mol_type = other DNA
agcacacattgctggttctgctcacgtcccgttgtattagccgccgctgtctcttatacacatctccgagcccacgagac

SEQ ID NO: 6 - DNA, 24 bp, synthetic construct
  source 1..24: mol_type = other DNA
  modified_base 1: mod_base = OTHER; note = The 5' terminus is modified with 6-Carboxyfluorescein.
cacgtctgactacaggcattccgt

SEQ ID NO: 7 - DNA, 24 bp, synthetic construct
  source 1..24: mol_type = other DNA
  modified_base 1: mod_base = OTHER; note = The 5' terminus is modified with a VIC fluorophore.
cacgtcaacctagtgcgacgatac

SEQ ID NO: 8 - DNA, 24 bp, synthetic construct
  source 1..24: mol_type = other DNA
  modified_base 1: mod_base = OTHER; note = The 5' terminus is modified with an ABY fluorophore.
cacgccgaggtctgtcacagtgta

SEQ ID NO: 9 - DNA, 24 bp, synthetic construct
  source 1..24: mol_type = other DNA
  modified_base 1: mod_base = OTHER; note = The 5' terminus is modified with a JUN fluorophore.
cacggtgaaccgtatctgacggca

SEQ ID NO: 10 - DNA, 24 bp, synthetic construct
  source 1..24: mol_type = other DNA
  modified_base 1: mod_base = OTHER; note = The 5' terminus is modified with a cyanine-5 fluorophore.
cacgtcccgttgtattagccgccg

SEQ ID NO: 11 - DNA, 26 bp, synthetic construct
  source 1..26: mol_type = other DNA
agcacacattgctggttctgctcacg

SEQ ID NO: 12 - DNA, 20 bp, synthetic construct
  source 1..20: mol_type = other DNA
gtctcgtgggctcggagatg

SEQ ID NO: 13 - DNA, 178 bp, synthetic construct
  source 1..178: mol_type = other DNA
aatgatacggcgaccaccgagatctacactcgtcggcagcgtcagatgtgtataagagacagnnnnagcacacattgctggttctgctcacgnnnnnnnnnnnnnnnnnnnnctgtctcttatacacatctccgagcccacgagacnnnnnnnnatctcgtatgccgtcttctgcttg

SEQ ID NO: 14 - DNA, 92 bp, synthetic construct
  source 1..92: mol_type = other DNA
aatgatacggcgaccaccgagatctacactcgtcggcagcgtcagatgtgtataagagacagnnnnagcacacattgctggttctgctcacg

SEQ ID NO: 15 - DNA, 80 bp, synthetic construct
  source 1..80: mol_type = other DNA
agcacacattgctggttctgctcacgnnnnnnnnnnnnnnnnnnnnctgtctcttatacacatctccgagcccacgagac

SEQ ID NO: 16 - DNA, 66 bp, synthetic construct
  source 1..66: mol_type = other DNA
ctgtctcttatacacatctccgagcccacgagacnnnnnnnnatctcgtatgccgtcttctgcttg

SEQ ID NO: 17 - DNA, 21 bp, synthetic construct
  source 1..21: mol_type = other DNA
agcacacattgctggttctgc

SEQ ID NO: 18 - DNA, 25 bp, synthetic construct
  source 1..25: mol_type = other DNA
aatgatacggcgaccaccgagatct

SEQ ID NO: 19 - DNA, 24 bp, synthetic construct
  source 1..24: mol_type = other DNA
caagcagaagacggcatacgagat

SEQ ID NO: 20 - DNA, 68 bp, synthetic construct
  source 1..68: mol_type = other DNA
caagcagaagacggcatacgagatcgctcagttcgtctcgtgggctcggagatgtgtataagagacag

SEQ ID NO: 21 - DNA, 68 bp, synthetic construct
  source 1..68: mol_type = other DNA
  modified_base 1: mod_base = OTHER; note = The 5' terminus is modified with a JUN fluorophore.
caagcagaagacggcatacgagatatatgagacggtctcgtgggctcggagatgtgtataagagacag

SEQ ID NO: 22 - DNA, 68 bp, synthetic construct
  source 1..68: mol_type = other DNA
caagcagaagacggcatacgagatcttatggaatgtctcgtgggctcggagatgtgtataagagacag

SEQ ID NO: 23 - DNA, 68 bp, synthetic construct
  source 1..68: mol_type = other DNA
caagcagaagacggcatacgagattaatctcgtcgtctcgtgggctcggagatgtgtataagagacag

SEQ ID NO: 24 - DNA, 68 bp, synthetic construct
  source 1..68: mol_type = other DNA
caagcagaagacggcatacgagatgcgcgatgttgtctcgtgggctcggagatgtgtataagagacag

SEQ ID NO: 25 - DNA, 116 bp, synthetic construct
  source 1..116: mol_type = other DNA
aatgatacggcgaccaccgagatctacactcgtggagcgtcgtcggcagcgtcagatgtgtataagagacagaggtcagatactgtctcttatacacatctccgagcccacgagac

SEQ ID NO: 26 - DNA, 116 bp, synthetic construct
  source 1..116: mol_type = other DNA
aatgatacggcgaccaccgagatctacacacattatccttcgtcggcagcgtcagatgtgtataagagacaggacgagattactgtctcttatacacatctccgagcccacgagac

SEQ ID NO: 27 - DNA, 116 bp, synthetic construct
  source 1..116: mol_type = other DNA
aatgatacggcgaccaccgagatctacactcgtggagcgtcgtcggcagcgtcagatgtgtataagagacagaacatcgcgcctgtctcttatacacatctccgagcccacgagac

SEQ ID NO: 28 - DNA, 116 bp, synthetic construct
  source 1..116: mol_type = other DNA
aatgatacggcgaccaccgagatctacacgtccacttgttcgtcggcagcgtcagatgtgtataagagacagctagtgctctctgtctcttatacacatctccgagcccacgagac

SEQ ID NO: 29 - DNA, 116 bp, synthetic construct
  source 1..116: mol_type = other DNA
aatgatacggcgaccaccgagatctacactggaacagtatcgtcggcagcgtcagatgtgtataagagacaggatcaaggcactgtctcttatacacatctccgagcccacgagac

SEQ ID NO: 30 - DNA, 116 bp, synthetic construct
  source 1..116: mol_type = other DNA
aatgatacggcgaccaccgagatctacacgttgatagtgtcgtcggcagcgtcagatgtgtataagagacaggactgagtagctgtctcttatacacatctccgagcccacgagac

SEQ ID NO: 31 - DNA, 116 bp, synthetic construct
  source 1..116: mol_type = other DNA
aatgatacggcgaccaccgagatctacacaccagcgacatcgtcggcagcgtcagatgtgtataagagacagagtcagacgactgtctcttatacacatctccgagcccacgagac

SEQ ID NO: 32 - DNA, 116 bp, synthetic construct
  source 1..116: mol_type = other DNA
aatgatacggcgaccaccgagatctacacgtgtggcgcttcgtcggcagcgtcagatgtgtataagagacaggagtcataggctgtctcttatacacatctccgagcccacgagac

SEQ ID NO: 33 - DNA, 116 bp, synthetic construct
  source 1..116: mol_type = other DNA
aatgatacggcgaccaccgagatctacacatcacgaaggtcgtcggcagcgtcagatgtgtataagagacagcttgccattactgtctcttatacacatctccgagcccacgagac

SEQ ID NO: 34 - DNA, 80 bp, synthetic construct
  source 1..80: mol_type = other DNA
agcacacattgctggttctgctcacgtatcgcccaagagcgcagtcctgtctcttatacacatctccgagcccacgagac

SEQ ID NO: 35 - DNA, 80 bp, synthetic construct
  source 1..80: mol_type = other DNA
agcacacattgctggttctgctcacgacgcaggatctaagccagcgctgtctcttatacacatctccgagcccacgagac

SEQ ID NO: 36 - DNA, 68 bp, synthetic construct
  source 1..68: mol_type = other DNA
caagcagaagacggcatacgagattatctgacctgtctcgtgggctcggagatgtgtataagagacag

SEQ ID NO: 37 - DNA, 68 bp, synthetic construct
  source 1..68: mol_type = other DNA
caagcagaagacggcatacgagatagagcactaggtctcgtgggctcggagatgtgtataagagacag

SEQ ID NO: 38 - DNA, 68 bp, synthetic construct
  source 1..68: mol_type = other DNA
caagcagaagacggcatacgagattgccttgatcgtctcgtgggctcggagatgtgtataagagacag

SEQ ID NO: 39 - DNA, 68 bp, synthetic construct
  source 1..68: mol_type = other DNA
caagcagaagacggcatacgagatctactcagtcgtctcgtgggctcggagatgtgtataagagacag

SEQ ID NO: 40 - DNA, 68 bp, synthetic construct
  source 1..68: mol_type = other DNA
caagcagaagacggcatacgagattcgtctgactgtctcgtgggctcggagatgtgtataagagacag

SEQ ID NO: 41 - DNA, 68 bp, synthetic construct
  source 1..68: mol_type = other DNA
caagcagaagacggcatacgagatgaacatacgggtctcgtgggctcggagatgtgtataagagacag

SEQ ID NO: 42 - DNA, 68 bp, synthetic construct
  source 1..68: mol_type = other DNA
caagcagaagacggcatacgagatcctatgactcgtctcgtgggctcggagatgtgtataagagacag

SEQ ID NO: 43 - DNA, 68 bp, synthetic construct
  source 1..68: mol_type = other DNA
caagcagaagacggcatacgagattaatggcaaggtctcgtgggctcggagatgtgtataagagacag

SEQ ID NO: 44 - DNA, 68 bp, synthetic construct
  source 1..68: mol_type = other DNA
caagcagaagacggcatacgagatgtgccgcttcgtctcgtgggctcggagatgtgtataagagacag

SEQ ID NO: 45 - DNA, 68 bp, synthetic construct
  source 1..68: mol_type = other DNA
caagcagaagacggcatacgagatcggcaatggagtctcgtgggctcggagatgtgtataagagacag

SEQ ID NO: 46 - DNA, 68 bp, synthetic construct
  source 1..68: mol_type = other DNA
caagcagaagacggcatacgagatgccgtaaccggtctcgtgggctcggagatgtgtataagagacag

SEQ ID NO: 47 - DNA, 68 bp, synthetic construct
  source 1..68: mol_type = other DNA
caagcagaagacggcatacgagataaccattctcgtctcgtgggctcggagatgtgtataagagacag

SEQ ID NO: 48 - DNA, 68 bp, synthetic construct
  source 1..68: mol_type = other DNA
caagcagaagacggcatacgagatggttgcctctgtctcgtgggctcggagatgtgtataagagacag

SEQ ID NO: 49 - DNA, 68 bp, synthetic construct
  source 1..68: mol_type = other DNA
caagcagaagacggcatacgagatctaatgatgggtctcgtgggctcggagatgtgtataagagacag

SEQ ID NO: 50 - DNA, 68 bp, synthetic construct
  source 1..68: mol_type = other DNA
caagcagaagacggcatacgagattcggcctatcgtctcgtgggctcggagatgtgtataagagacag

SEQ ID NO: 51 - DNA, 24 bp, synthetic construct
  source 1..24: mol_type = other DNA
cacgtatcgcccaagagcgcagtc

SEQ ID NO: 52 - DNA, 24 bp, synthetic construct
  source 1..24: mol_type = other DNA
  modified_base 1: mod_base = OTHER; note = The 5' terminus is modified with a cyanine-5 fluorophore.
cacgacgcaggatctaagccagcg

SEQ ID NO: 53 - DNA, 29 bp, synthetic construct
  source 1..29: mol_type = other DNA
aatgatacggcgaccaccgagatctacac

(19) United States
(12) Patent Application Publication
(10) Pub. No.: US 2026/0258475 A1
(43) Pub. Date: Sep. 3, 2026

(54) ANALYTE QUANTIFICATION WITH BEAD AND IMMUNO qPCR-BASED ELISA (BIQ-ELISA)

(71) Applicant: RayBiotech Life, Inc., Peachtree Corners, GA (US)

(72) Inventors: Ying-Qing Mao, Peachtree Corners, GA (US); Ruo-Pan Huang, Peachtree Corners, GA (US); Tao Wang, Peachtree Corners, GA (US); Xuelin Wang, Peachtree Corners, GA (US)

(21) Appl. No.: 19/462,372

(22) Filed: Jan. 28, 2026

Related U.S. Application Data
(60) Provisional application No. 63/765,018, filed on Feb. 28, 2025.

(51) Int. Cl. C12Q 1/6804; G01N 33/68

(52) U.S. Cl. CPC C12Q 1/6804; G01N 33/6863; G01N 33/6866; G01N 2333/525; G01N 2333/54

(57) ABSTRACT

Disclosed are kits and methods for detecting target analytes in a sample using modified magnetic particles and nucleic acid barcodes. The kits include magnetic particles conjugated to a first binding agent and a second binding agent conjugated to a nucleic acid barcode, with the magnetic particles and binding agents along with target antigens form immune complex. The nucleic acid barcode is amplified using qPCR and/or sequenced using Next Generation Sequencing (NGS). Also disclosed are methods for forming immune complexes, performing signal amplification, and determining analyte concentrations.

### CROSS-REFERENCE TO RELATED APPLICATIONS

This application claims priority to and the benefit of U.S. Provisional Application No. 63/765,018, filed on Feb. 28, 2025, which is incorporated herein and by reference in its entirety.

### REFERENCE TO THE SEQUENCE LISTING

The Sequence Listing submitted as an XML file named “RAYB_100_US_ST26.xml,” created on Jan. 9, 2026, and having a size of 54,240 bytes is hereby incorporated by reference pursuant to 37 C.F.R. § 1.834(c)(1).

### FIELD OF THE INVENTION

The disclosed invention is generally directed towards detection of analytes using binding agents, as well as related kits and methods of using thereof.

### BACKGROUND OF THE INVENTION

Traditional ELISA has remained the gold standard for protein detection for several decades due to the high affinity and specificity of antibody-antigen recognition. ELISA can detect picomolar level of protein concentrations, making it suitable for detecting small amounts of proteins in complex samples like blood or urine. However, many biomarkers of interest exist at ultralow concentrations (in the low picomolar or femtomolar range), which often fall below the limit of detection (LOD) of current diagnostic methods. As a result, proteins present at femtomolar concentrations can escape detection, creating an important gap in diagnostic capabilities. This limitation is especially problematic in the early detection of diseases like cancer or neurological disorders, where early-stage biomarkers are often present in minute quantities.

One way to boost detection sensitivity is through immune-PCR. Instead of directly labeling the detection antibody with an enzyme as in ELISA, antibodies are linked to a DNA tag, which serves as a template for PCR amplification. Taking advantage of the high specificity of antibody-based protein detection with the amplification power of PCR, Immune-PCR can detect extremely low levels of biomarkers. Two of the most widely used methods in this category are the Proximity Ligation Assay (PLA) and the Proximity Extension Assay (PEA). In both systems, two antibodies are designed to target different epitopes of the same antigen, each conjugated to distinct DNA oligonucleotides. When these antibodies bind to the same target protein, the DNA strands come into close proximity, triggering a reaction that facilitates signal amplification via PCR. PLA utilizes external linker and DNA ligase to join the two oligonucleotide strands into a single DNA sequence for PCR amplification. Whereas PEA relies on direct hybridization and DNA polymerase to extend the overlapping sequences.

Both PLA and PEA took the approach of homogeneous assay format. Homogeneous assays do not require any washing or separation steps; all reactions occur in the same solution phase, which makes the protocol faster and easier to perform, and can be easily automated. However, due to the presence of unbound molecules in the same solution, homogeneous assays tend to have high background noise and lower sensitivity than solid-phase assays. On the contrary, traditional protein detection adopts the solid surface assay approach, where one of the assay components (e.g., an antibody or antigen) onto a solid phase, like a microplate or beads. The solid surface allows for thorough washing to remove unbound or non-specifically bound molecules, resulting in lower background noise and increased sensitivity. Solid phase PEA of fixing the immunocomplex through a third antigen binding antibody has been proved to boost the detection sensitivity to SIMOA (Single Molecule Array) level. SIMOA achieves its extraordinary sensitivity by isolating individual enzyme-labeled immunocomplexes into thousands of microscopic wells, each well effectively acting as a tiny independent assay. This isolation allows SIMOA to detect extremely low concentrations of proteins, reaching sensitivity levels in the femtogram per milliliter (fg/mL) range. This sensitivity can be up to 1000 times higher than traditional ELISA, making SIMOA one of the most advanced technologies for detecting low-abundance proteins in research and clinical diagnostics. Microscopic beads have been extensively used as the solid-phase support for immunoassays due to the many advantages they offer, including enhanced sensitivity through increased surface area, faster reaction kinetics due to uniform suspension in the liquid phase, reduced sample volume, multiplexing capabilities, and automation compatibility. Magnetic beads provide the additional benefit of simplifying the separation of bound antibodies or antigens from complex mixtures by applying a magnetic field. This easy separation minimizes the need for multiple washing steps, reduces background noise, and further enhances the assay's sensitivity and specificity. Two of the most widely used bead-based immunoassays are the Luminex Bead-Based Multiplex Assays and the Flow Cytometry-Based Bead Assays. Luminex xMAP Assays utilize uniquely colored beads for multiplexing and a common fluorescent dye for signal detection, allowing simultaneous measurement of multiple analytes within the same sample. Like Luminex, BD Biosciences' Cytometric Bead Array (CBA) employs fluorescently labeled beads for multiplexing but utilizes the precision of flow cytometry for the readout. Both xMAP and CBA offer similar detection sensitivity to ELISA but with multiplexing capabilities.

There remains a need for compositions and tests that can detect low concentrations of analytes with high sensitivity and specificity.

Therefore, it is the object of the present invention to provide kits and methods of using the kits that can detect low concentrations of analytes with high sensitivity and specificity.

### BRIEF SUMMARY OF THE INVENTION

Kits for detection of one or more types of target analyte in a sample, and methods of use thereof, are provided.

In some forms, the kit is used to detect one type of analyte in a sample and it includes a pair of binding agents specific for the analyte, which bind to different epitopes on the analyte or to different parts of the analyte. In these forms, the kit includes a first reagent including magnetic particles, modified with a plurality of a first binding agent specific for the analyte; and a second reagent including a plurality of a second binding agent specific for the analyte, each conjugated to a nucleic acid barcode. The first binding agent can be bound to the particle via any suitable interactions, such as via an affinity binding pair (e.g., a biotin-streptavidin binding pair). The magnetic particles preferably have a size in the micrometer range. In some forms, the kit is used for detecting more than one type of analyte in a sample, and it includes first binding agents specific for each type of analyte and second binding agents specific for each type of analyte. In these forms, second binding agents specific for different types of analytes are labeled with different nucleic acid barcodes, such that each nucleic acid barcode corresponds to one specific type of analyte.

The first and second binding agents include any binding agent pair that specifically bind to the analyte of interest and preferably show reduced or no cross reactivity with other analytes. When the particles are modified with a first member of an affinity binding pair, a second member of the affinity binding pair is also provided in the kit in any suitable form. For example, a labeling reagent containing the second member is provided as part of the kit, where the labeling reagent can react with a functional group of the analyte(s) of interest to label the analyte(s) with the second member (also referred to herein as “labeled analyte(s)”). The labeled analyte(s) can then bind with the first member of the affinity binding pair modified on the particles. The second binding agent is conjugated to a DNA barcode which allows subsequent identification of the analyte is bound.

In some forms, the kit is used to detect one or more types of analytes in a sample and it includes only one binding agent specific for the analyte or each analyte (when more than one type of analytes are being detected), which binds to an epitope on the analyte or each analyte. In these forms, the kit includes a first reagent including magnetic particles, modified with a plurality of a member (e.g., streptavidin) of an affinity binding pair; and a second reagent including a plurality of a second binding agent specific for the analyte or each analyte, each conjugated to a nucleic acid barcode. When more than one type of analyte is being detected, the second binding agent specific for each analyte contains a unique nucleic acid barcode conjugated thereto, corresponding to the specific type of analyte. In these forms, the kit can optionally include a protein labeling reagent that reacts with all proteins in the sample and universally labels the proteins with a complementary member (e.g., biotin) of the affinity binding pair to facilitate binding with the modified magnetic particles.

In some forms, particles other than magnetic particles in the first reagent can be used. Such particles include, but are not limited to, polystyrene beads, silica beads, latex beads, hydrogel beads, gold nanoparticles (AuNPs), quantum dots, or glass beads. These particles are modified with a plurality of a first binding agent specific for the analyte or a plurality of a first member of a pair of affinity binding pairs.

Also provided are methods of detecting one or more types of analytes in a sample using the disclosed kits. In some forms, the method includes contacting a sample with the first and second reagent to form an immunocomplex of the analyte(s) of interest in the sample, with its first and second binding agents. In some other forms, the method includes labeling proteins in the sample with a member of an affinity binding pair and then contacting the sample with the first reagent containing magnetic particles modified with a complementary member of the affinity binding pair and the second reagent to form an immunocomplex between the analyte(s) and the second binding agent in the second reagent. In either of these forms, the method can further include releasing the immunocomplex from the magnetic particle using a suitable elution buffer and using the DNA barcode as the template for PCR (for example qPCR) and optionally, NGS (Next Generation Sequencing) to identify the analyte(s), based on the DNA barcodes.

### BRIEF DESCRIPTION OF THE DRAWINGS

FIGS. 1A-1C illustrate a schematic representation of the workflow for the Bead and Immuno qPCR-based ELISA (BIQ-ELISA) assay, where “CAB” refers to capture antibody; and “ONA” refers to oligonucleotide acid; DAB refers to detection antibody.

FIG. 2 is a plot comparing Single Molecule Array (SIMOA) technology with BIQ ELISA for detecting TNFa, where the X-axis represents patient sample numbers and the Y-axis represents amounts (pg/ml) of TNF-alpha detected.

FIG. 3 is a schematic of primer design for BIQ system. P5, P7, Rd1SP, Rd2SP, and i7 are Illumina sequencing components; SP_F: Unique BIQ-ELISA sequencing primer, with 4 random bp at 5′-end for NGS, and 4 common bp at 3-end for TaqMan; ONA: 20 bp unique DNA barcode; matching TaqMan probes are designed based upon this. Primer 2 alone is used for individual BIQ-ELISA. Primer 1 & Primer 3 is used for NGS library construction.

FIGS. 4A-4D shows the results of crosslinking reaction using a crosslinker, BS3. FIG. 4A is a chemical structure of BS3. FIG. 4B are gel images showing protein stain and DNA stain. Lane 1: DSS conjugation via activating DNA; Lane 2: BS3 conjugation via activating DNA; Lane 3: BS3 activated streptavidin; Lane 4: BS3 conjugation via activating streptavidin. FIG. 4C are gel images showing BS3 conjugation with P7 end fragment in 200 mM HEPES. Lane 1: conjugates by P7 end fragment activated by 50× molar excess BS3; Lane 2: conjugates by P7 end fragment activated by 100× molar excess BS3; Lane 3: conjugates by P7 end fragment activated by 250× molar excess BS3; Lane 4: conjugates by P7 end fragment activated by 100× molar excess BS3, 3× molar excess of DNA to streptavidin; Lane 5: conjugates by P7 end fragment activated by 100× molar excess BS3, 6× molar excess of DNA to streptavidin; Lane 6: conjugates by P7 end fragment activated by 100× molar excess BS3, 9× molar excess of DNA to streptavidin. FIG. 4D is a gel image showing mouse IgG conjugation with different DNA ratios. The pH was further evaluated, and the efficiency was greatly improved.

FIGS. 5A-5D shows results of streptavidin beads test. FIGS. 5A-5B represent 1) HV0150 Hi-SurMag Streptavidin, 150 nm, 2 ml, 1 mg/ml (Custom), 2) MV1000-002 MonoMag Streptavidin, 1 um 2 ml (Custom), 3) HV1000-02 HiSur Streptavidin, 1 um, 2 ml (Custom), 4) 2574067 1 mL Dynabeads™ M-280 Streptavidin, 2.8 μm, 5) 01342559 1 mL Dynabeads™ M-270 Streptavidin, 2.8 μm, 6) 01342859 1 mL Dynabeads™ MyOne™ Streptavidin C1, 1 um, 7) 01340664 1 mL Dynabeads™ MyOne™ Streptavidin T1, 1 μm. FIGS. 5C-5D represent 9 different streptavidin beads tested in PCR reactions: 1) HV0150 Hi-SurMag Streptavidin, 150 nm, 2 ml, 1 mg/ml (Custom), 2) MV1000-002 MonoMag Streptavidin, 1 um 2 ml (Custom), 3) HV1000-02 HiSur Streptavidin, 1 um, 2 ml (Custom), 4) 2574067 1 mL Dynabeads™ M-280 Streptavidin, 2.8 μm, 5) 01342559 1 mL Dynabeads™ M-270 Streptavidin, 2.8 μm, 6) 01342859 1 mL Dynabeads™ MyOne™ Streptavidin C1, 1 um, 7) 01340664 1 mL Dynabeads™ MyOne™ Streptavidin T1, 1 um, 8) Bioeast M2800S3-XC 2.8 μm, 9) Beaver 22307-1 1 μm. Dynabeads M-280 is identified as the most suitable for downstream PCR.

FIGS. 6A-6J are plots showing comparison of detection sensitivity between filter plate methods (FIGS. 6A, 6C, 6E, 6G, 6I) and magnetic beads washer methods (FIGS. 6B, 6D, 6F, 6H, 6J) for IL-1b (FIG. 6A-6B), TNFa (FIG. 6C-6D), IL-12p70 (FIG. 6E-6F), IFNg (FIG. 6G-6H), IL-6 (FIG. 6I-6J).

FIG. 7 is a pair of plots showing reactions in 10 ul volume (Sul sample+5 ul bead mix) have the better consistency and lowest CV.

FIG. 8A is a schematic of barcode and primer design for Next Generation Sequencing. FIG. 8B shows bioanalyzer results of the pooled samples. The concentration of the pooled sample is 1.1 ng/ul and is ready for sequencing.

FIG. 9 is a schematic showing the BIQL assay.

FIG. 10A is a bar graph showing the cycle threshold (CT) values for IFNg detection between the BIQ and BIQL assays. FIG. 10B is a bar graph showing the cycle threshold (CT) values for TNFa detection between the BIQ and BIQL assays. FIG. 10C is a bar graph showing the cycle threshold (CT) values for IL-1b detection between the BIQ and BIQL assays. The X-axis of the bar graphs represents serial antigen dilutions (1-8) with 9 as the negative control.

FIG. 11 is a plot showing the CT value for different concentrations of IL-6 between BIQL assays detected with either TaqMan probes or SYBR Green.

FIG. 12 is a schematic showing methods 1~4 for NGS amplicon library construction.

FIG. 13 is a table containing exemplary sequences for DNA barcodes used for the detection of ALPP, CD38, IL-8, SOD1, and VCAN.

FIG. 14 is a table containing a list of primer sequences with different sample indices for the detection of ALPP, CD38, IL-8, SOD1, and VCAN.

FIG. 15 is a table containing a list of sequences including additional Illumina sequencing components: Read 2 Sequencing Primer (Rd2SP, 34 bp), i7 index (8 bp, used for sample indexing), and P7 adapter (24 bp).

FIG. 16 is a table displaying the barcode copy numbers counted by NGS detection using the BIQL assay.

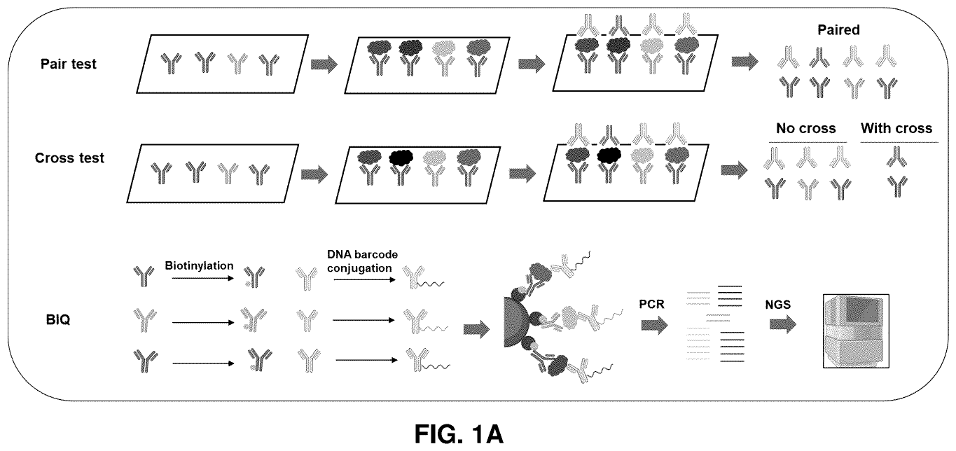

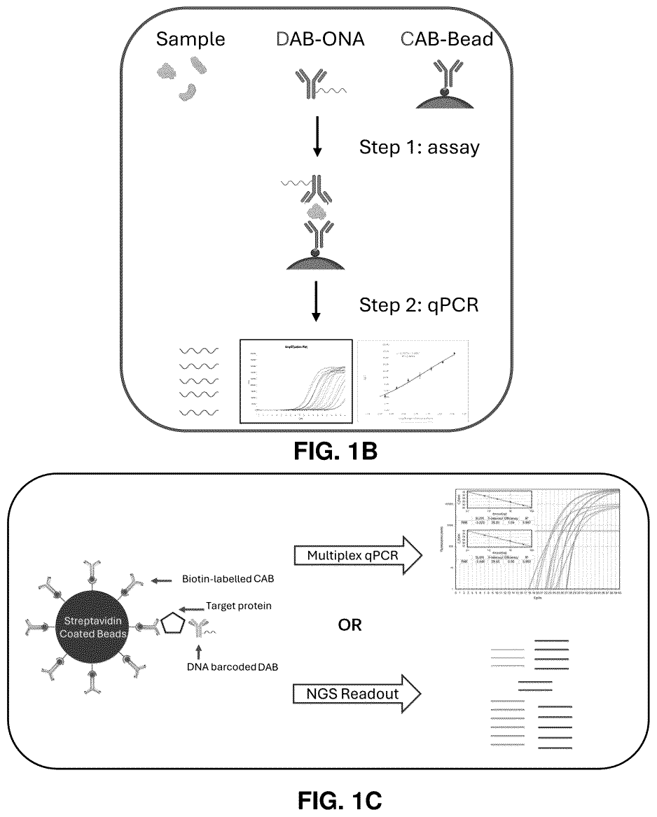

### DETAILED DESCRIPTION OF THE INVENTION

### A. Definitions

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multi-specific antibodies (e.g., bispecific antibodies), and antibody fragments so long as they specifically bind the target antigen. Any specific antibody can be used in the methods and compositions provided herein. Thus, in some forms the term “antibody” encompasses a molecule including at least one variable region from a light chain immunoglobulin molecule and at least one variable region from a heavy chain molecule that in combination form a specific binding site for the target antigen. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia, C. et al. (1987) “Canonical Structures For The Hypervariable Regions Of Immunoglobulins,” J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies of the invention). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass. An “antibody fragment” or “antigen binding fragment” of an antibody is defined as at least a portion of the variable region of the immunoglobulin molecule that binds to its target, i.e., the antigen binding region (also antigen binding domain). In some forms it specifically covers single antibodies and clones thereof and anti-antibody compositions with polyepitopic specificity. The antibody of the present methods and compositions can be monoclonal or polyclonal. An antibody can be in the form of an antigen binding antibody fragment including a Fab fragment, F(ab′) 2 fragment, a single chain variable region, and the like. Fragments of intact molecules can be generated using methods well known in the art and include enzymatic digestion and recombinant means. Thus, the “fragment” may be a recombinant protein, e.g., a fusion protein.

As used herein, any form of the “antigen” can be used to generate an antibody that is specific for the target antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In some forms, the antibody of the methods and compositions herein specifically bind at least a portion of the extracellular domain of the target antigen of interest. In some forms, the binding fragments useful in the present invention are biologically active fragments. As used herein, the term “biologically active” refers to an antibody or antibody fragment that is capable of binding the desired the antigenic epitope and directly or indirectly exerting a biologic effect.

“Bispecific” antibodies are also useful in the present methods and compositions. As used herein, the term “bispecific antibody” refers to an antibody, typically a monoclonal antibody, having binding specificities for at least two different antigenic epitopes. In some forms, the epitopes are from the same antigen. In another form, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The term “specifically binds” or “immuno-specifically binds” refers to the binding of an antibody to its cognate antigen, while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The term “monoclonal antibody” or “mAb” refers to an antibody obtained from a substantially homogeneous population of antibodies, i.e., the individual antibodies within the population are identical except for possible naturally occurring mutations that may be present in a small subset of the antibody molecules.

As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, the terms “oligonucleotide” or a “polynucleotide” are synthetic or isolated nucleic acid polymers including a plurality of nucleotide subunits.

The terms “contact”, “contacting” or “bringing into contact” describe placement in physical association for example, in solid and/or liquid form. For example, contacting or combining can occur in vitro with one or more primers and/or probes and a biological sample (such as a sample including nucleic acids) in solution.

An “aptamer” refers to a nucleic acid molecule that is capable of binding to a particular molecule of interest with high affinity and specificity (Tuerk and Gold, Science 249:505 (1990); Ellington and Szostak, Nature 346:818 (1990)). The binding of a ligand to an aptamer, which is typically RNA, changes the conformation of the aptamer and the nucleic acid within which the aptamer is located. The conformation change inhibits translation of an mRNA in which the aptamer is located, for example, or otherwise interferes with the normal activity of the nucleic acid. Aptamers may also be composed of DNA or may comprise nonnatural nucleotides and nucleotide analogs. An aptamer will most typically have been obtained by in vitro selection for binding of a target molecule. However, in vivo selection of an aptamer is also possible.

“Amplification” or “amplifying” refers to increasing the number of copies of a nucleic acid molecule, such as a gene, fragment of a gene, or other genomic region. The products of an amplification reaction are called amplification products or amplicons.

“Isolated,” “isolating,” “purified,” “purifying,” “enriched,” and “enriching,” when used with respect to nucleic acids of interest (e.g., DNA such as intact or fragmented genomic DNA, amplicons, etc.), indicate that the nucleic acids of interest at some point in time were separated, enriched, sorted, etc., from or with respect to other cellular material to yield a higher proportion of the nucleic acids of interest compared to the other cellular material, contaminates, or active agents such as enzymes, proteins, detergent, cations or anions. “Highly purified,” “highly enriched,” and “highly isolated,” when used with respect to nucleic acids of interest, indicates that the nucleic acids of interest are at least about 70%, about 75%, about 80%, about 85%, about 90% or more, about 95%, about 99% or 99.9% or more purified or isolated from other cellular materials, contaminates, or active agents such as enzymes, proteins, detergent, cations or anions. “Substantially isolated,” “substantially purified,” and “substantially enriched,” when used with respect to nucleic acids of interest, indicate that the nucleic acids of interest are at least about 70%, about 75%, or about 80%, more usually at least 85% or 90%, and sometimes at least 95% or more, for example, 95%, 96%, and up to 100% purified or isolated from other cellular materials, contaminates, or active agents such as enzymes, proteins, detergent, cations or anions.

As used herein, the terms “complement”, “complementary”, and “complementarity” with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5”. The second sequence can be referred to as the reverse complement of the first sequence, and the first sequence can be referred to as the reverse complement of the second sequence.

As used herein, the term “hybridize” refers to a process where two substantially complementary or complementary nucleic acid strands anneal to each other under appropriately stringent conditions to form a duplex or heteroduplex through formation of hydrogen bonds between complementary base pairs.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary or complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA. Primers are typically at least 10, 15, 18, or 30 nucleotides in length or up to about 100, 110, 125, or 200 nucleotides in length. In some forms, primers are between about 15 to about 60 nucleotides in length, and or between about 25 to about 40 nucleotides in length. In some forms, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” or “primer set” refers to a forward and reverse primer pair (i.e., a left and right primer pair) that can be used together to amplify a given region of a nucleic acid of interest.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

Multiplexing, as used herein, refers to a process that allows simultaneous analysis of multiple analytes and/or multiple samples in a single assay or sequencing run. This is achieved through the use of unique molecular identifiers, such as nucleic acid barcodes, that allow for the discrimination and identification of different analytes and/or samples after data collection.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### B. Kits

Described herein are kits for detecting one or more types of target analyte in a sample.

In some forms the kit is a Bead-based Immuno Quantitative Enzyme Linked ImumunoSorbent Assay (BIQ-ELISA) which is a protein detection method that merges the specificity of sandwich-based ELISA, the sensitivity of real-time PCR, and the easy manipulation of magnetic microspheres. In this system, the first binding agent is biotin-labelled and pre-absorbed onto streptavidin-coated beads, while the second binding agent is conjugated with a unique oligonucleotide acid (ONA) barcode for signal amplification.

This technology represents an advancement in protein detection assays, providing an ultrasensitive and highly specific method for quantifying proteins in biological samples. This technology's ability to combine multiple advantages of existing methods into a single, easy-to-use platform makes it an important tool for the research community. The BIQ-ELISA kits can be used for the quantitative measurement of proteins in various sample types including serum, plasma, and cell culture supernatants with femtogram per milliliter detection sensitivity.

The features of the BIQ-ELISA kits include, but not limited to, sensitivity (up to 1,000× more than traditional ELISA); low sample volume (less than 10 μL); specificity (sandwich-based detection); simplicity (two steps processing); efficiency (data generated within 4 hours); Multiplexing (designed for panels of up to five or more targets).

In some forms, the kit does not use sandwich-based protein detection, as the second binding agent is used without a first binding agent. In some forms, the kit without a first binding agent is a Bead-based Immuno-qPCR with biotin-labeled sample (BIQL) kit. In the BIQL kit, biotin is attached to target analytes, which interact directly with streptavidin-modified magnetic particles.

In these forms, the analyte is labeled with a protein labeling reagent that contains a member of an affinity pair. The protein labeling reagent reacts with the analyte to attach the member of an affinity pair to facilitate interaction with magnetic particles that are modified with the complementary member of the affinity pair. In these forms, the second binding agent with a unique ONA barcode as mentioned above is used to bind directly to the target analyte for detection. For example, in such systems, all proteins in the sample are first biotinylated. These biotinylated proteins are immobilized onto streptavidin-coated magnetic beads and then incubated with an array of DNA-barcoded detection antibodies to form highly specific immunocomplexes between the analyte and the second binding agent. Such kits can also be used for the quantitative measurement of proteins in various sample types including serum, plasma, and cell culture supernatants with femtogram per milliliter detection sensitivity. These kits also facilitate analyte detection using low sample volume (less than 10 μL) to detect one or more analytes, more than 100 analytes, or more than 1000 analytes.

Unlike traditional sandwich assays that require a pair of antibodies, this platform leverages direct quantification via qPCR or next-generation sequencing (NGS), using the DNA tags as unique molecular identifiers. For example, BIQL integrates the femtogram-level sensitivity of bead-based immuno-qPCR (BIQ) with the scalable multiplexing power of biotin-labeled sample arrays. By combining direct biotinylation of proteins, magnetic bead capture, and DNA-barcoded antibody detection, BIQL facilitates digital, highly specific quantification of up to 1000+ proteins per run—ideal for clinical biomarker discovery, systems biology, and next-generation diagnostics.

### 1. First Reagent Including Modified Magnetic Particles

i. Structure

Structurally, in some forms, the first reagent includes magnetic particles associated with a binding agent via an affinity binding pair. In some other forms, the first reagent includes magnetic particles modified with a member of an affinity binding pair without a first binding agent.

a. Magnetic Particles

Disclosed herein are modified magnetic particles for solid support. In some forms, the magnetic particles are modified with a first binding agent, which is bound to the magnetic particles via an affinity binding pair. For example, the magnetic particles are first modified with a first member (e.g., streptavidin) of an affinity binding pair on their surface, which then interact with a complementary member (e.g., biotin) of the affinity binding pair on the first binding agent to produce the magnetic particles modified with the first binding agent. In some forms, the magnetic particles are modified with a member (e.g., streptavidin) of an affinity binding pair, which is available for binding with proteins labeled with a complementary member (e.g., biotin) of the affinity binding pair. Magnetic particles modified with a member of an affinity binding pair, such as magnetic particles modified with streptavidin, are commercially available (such as those from Dynabeads, Sera-Mag, MagniSort, BioMag, MagMAX, Pure Proteome, Adembeads, Pierce™, etc.), or prepared using chemistry known in the art (such as those described in Wang, Tao, et al., ACS Omega. 9(49):47951-47963 (2024); Zhang, Z., et al., SCI CHINA SER B 50, 127-134 (2007); Gong, P., et al., J Nanopart Res 15, 1558 (2013)).

In some forms, solid support can include any solid material to which components of the assay can be adhered or coupled. Examples include, but are not limited to, materials such as acrylamide, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates can have any useful form including thin films or membranes, beads, bottles, dishes, fibers, woven fibers, shaped polymers, particles and microparticles. Some forms of substrates are plates and beads. A useful form of beads is magnetic beads.

In some forms, particles other than magnetic particles can be used in the first reagent. Such particles include, but are not limited to, polystyrene beads, silica beads, latex beads, hydrogel beads, gold nanoparticles (AuNPs), quantum dots, or glass beads. These particles are modified to include the binding agent for binding with the analyte.

b. Affinity Binding Pair

In some forms, the members of the affinity binding pair (also referred to herein as “binding pairs”) are selected from biotin, 2-iminobiotin, avidin, streptavidin, neutravidin, glutathione, glutathione s-transferase, maltose, maltose-binding protein, intein, chitin, and chitin-binding protein.

Other suitable binding pairs include, receptor/ligand pairs, antibodies/antigens, natural or synthetic receptor/ligand pairs, hapten/antibody pairs, antigen/antibody pairs, epitope/antibody pairs, mimitope/antibody pairs, aptamer/target molecule pairs, hybridization partners, intercalater/target molecule pairs, and the use of a surface and anchoring reagent bound by electrostatic charge.

For the biotin-avidin affinity binding pair, such as biotin-streptavidin or biotin-NeutrAvidin pair, biotin can be attached to the first binding agent and streptavidin, avidin or NeutrAvidin may be attached to the carrier or surface, or vice versa. In some forms, biotin binds via affinity interactions to biotin-binding compounds, thereby non-covalently conjugating the first binding agent to the carrier or surface. In some forms, other affinity pairs used for immobilizing first binding agent to solid surface include but are not limited to His-tag/Ni²⁺ or Co²⁺, Strep-tag/Strep-Tactin®, Protein A/IgG, Protein G/IgG, SBP-tag/Streptavidin, FLAG-tag/Anti-FLAG antibody resin, HA-tag/Anti-HA antibody resin, Myc-tag/Anti-Myc antibody resin, Halo-tag/HaloTag ligand, SpyTag/SpyCatcher.

In some other forms, when the proteins in a sample are labeled with a member of an affinity pair and thereby directly bind to a complementary member of the affinity pair on the surface of the carrier, the member and complementary member of the affinity pair can be any of those described above, such as biotin and avidin. For example, the proteins in the sample are labeled with biotin, which in turn bind to streptavidin on the surface of a carrier, such as streptavidin modified magnetic particles.

Non-covalent linkage can be carried out through electrostatic interactions, hydrogen bonding interactions, metal coordination, physical adsorption, host-guest interactions, hydrophobic interactions, π-stacking interactions, van der Waals interactions, magnetic interactions, and dipole-dipole interactions.

A preferred non-covalent linkage is provided by the affinity interactions involved receptor-ligand complex formation. Binding of a ligand to its binding partner can occur by intermolecular forces, such as ionic bonds, hydrogen bonds, hydrophobic interactions and Van der Waals forces. Thus, affinity interactions as used herein refers to the combination of non-covalent interactions between a ligand and its binding partner to form a complex.

c. Binding Agent

Disclosed herein are the first and second binding agents that are independently an antibody, an aptamer, or a peptide, or a combination thereof. Typically, the first binding agent is bound to the particle via an affinity binding pair and present on the surface of the particle, facing outward to facilitate binding with an analyte of interest. In some forms, the kit only includes a second binding agent that is specific for an analyte of interest bound to the particle, such as via an affinity binding pair. The second binding agent is typically conjugated to a nucleic acid barcode.

In some forms, suitable first and second binding pairs, such as antibody binding pairs, can be identified using following steps: 1. Identify Suitable Antibody Pairs: Perform dot-blot screening by testing each antibody as both a capture and detection antibody. Rank antibody combinations based on signal intensity, as higher intensity suggests stronger binding affinity to the target protein. Select the highest-affinity pairs for further sensitivity testing; and 2. Check for Cross-Reactivity: Print all capture antibodies on a single array. Test each target protein with its corresponding detection antibody separately. Analyze the array data to determine specificity: Non-cross-reactive pairs will show strong signals only for their intended antigen. Cross-reactive pairs will exhibit significant binding to unintended targets. Select the final antibody pairs that exhibit minimal to no cross-reactivity.

The orientation of the binding agent is generally upward and exposed to the surrounding environment to facilitate binding with target agents, such as analytes. These binding agents are designed and oriented in a manner that increases their ability to interact with and bind to target agents, such as analytes, present in an environment, typically a liquid medium such as buffers, biological fluids, or other aqueous solutions. This spatial arrangement enhances accessibility and interaction between binding agents and target analytes. In some forms, by maintaining an upward facing and exposed configuration, the binding agents are prevented from being obscured by other components, surface structures, or steric hindrance that might otherwise reduce binding efficiency.

### (A) Antibody

In some forms, the first and the second binding agent is each an antibody. In some forms the analyte is selected from the group consisting of antibodies specific for IL-8 (Interleukin-8), ALPP (Alkaline phosphatase for example, placental type (EC 3.1.3.1)), CD38 (Cluster of Differentiation 38), SOD1 (Superoxide dismutase-1), and VCAN (Versican) and the DAB and CAB antibodies are disclosed herein in Table 2.

Thus, in some forms, the IL-8-specific DAB (DNA barcoded) is the anti-IL8 antibody supplied by RayBiotech, (Peachtree Corners, GA) under catalog number 130-10975, and the binding pair antibody. Thus, in some forms, the ALPP-specific DAB (DNA barcoded) is the anti-ALPP antibody supplied by RayBiotech, (Peachtree Corners, GA) under catalog number 130-10518, and the binding pair antibody is supplied under catalog number 130-10519B. Thus, in some forms, the CD38-specific DAB (DNA barcoded) is the anti-CD38 antibody supplied by RayBiotech, (Peachtree Corners, GA) under catalog number 130-10323, and the binding pair antibody is supplied under catalog number 130-10324B. Thus, in some forms, the SOD1-specific DAB (DNA barcoded) is the anti-SOD1 antibody supplied by RayBiotech, (Peachtree Corners, GA) under catalog number 130-10369, and the binding pair antibody is supplied under catalog number 130-10366B. Thus, in some forms, the VCAN-specific DAB (DNA barcoded) is the anti-VCAN antibody supplied by RayBiotech, (Peachtree Corners, GA) under catalog number 130-10778 and 130-10773.

Native antibodies are usually heterotetrameric glycoproteins, composed of two identical light chains (LC) and two identical heavy chains (HC). LC Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H) or VH) followed by a number of constant domains. Each light chain has a variable domain at one end (V(L) or VL) and a constant domain at its other end; the constant domain of the light chain is aligned with the first constant domain of the heavy chain, and the light chain variable domain is aligned with the variable domain of the heavy chain. Particular amino acid residues are understood to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

In the context of antibodies and fragments thereof, the terms “variable region,” “variable sequence,” and the like, are used to describe certain portions of the variable domains that differ in sequence among antibodies and are used in the binding and specificity of each particular antibody for its particular antigen. However, the variability is not evenly distributed through the variable domains of antibodies. It is typically concentrated in three segments called complementarity determining regions (CDRs) or hypervariable (HV) regions both in the light chain and the heavy chain variable domains. The more highly conserved portions of the variable domains are called the framework (FR). The variable domains of native heavy and light chains each comprise four FR regions FR1, FR2, FR3, and FR4), largely adopting a β-sheet configuration, connected by three CDRs (HV1, HV2, HV3), which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). CRDs are typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, MD. (1991)). Residues that form core “hypervariable loops” are typically at approximately residues 26-32 (L1), 50-52 (L2) and 91-96 (L3) in the light chain variable domain and 26-32 (H1), 53-55 (H2) and 96-101 (H3) in the heavy chain variable domain (Chothia and Lesk, 1987, J. Mol. Biol. 196:901-917). The constant domains are not involved directly in binding an antibody to an antigen, but exhibit various effector functions, such as participation of the antibody in antibody-dependent cellular toxicity.

It is well-known that the variable regions of antibodies—and, in particular, the complementarity determining regions (CDRs) of the variable regions—are primarily responsible for the binding and binding specificity of antibodies. It is also well-known that portions of antibodies other than the variable regions (or other than the CDRs) can be substituted, altered, eliminated, etc. without abolishing the binding and binding specificity of the antibodies (or antibody fragments in the case of elimination of portions of the antibody). The well-known modular nature of antibody structure allows extensive substitution, alteration, elimination, etc. of portions of antibodies other than the variable regions (or other than the CDRs) while retaining the binding and binding specificity of the variable regions and CDRs. For example, the antibodies and antibody fragments can be in any form of antibody binding fragments that contains any one or all of the CDR sequences of the B5 antibody and any form of antibody binding fragments that contains the CDR sequences of the B5 antibody. Such principals have been amply demonstrated by production and use of chimeric antibodies, recombinant antibodies, humanized antibodies, and the numerous types of antibody fragments and antibody-derived polypeptides, such as F(ab′)2, fragment antigen-binding (Fab), half antibodies, single-chain variable fragments (scFv), VhH domain, V-NAR domain, VH domain, VL domain, F(ab)3, bis-scFv, diabody, triabody, tetrabody, and minibody (Hollinger and Hudson, Nature Biotech. 23(9):1126-1136 (2005) (and references cited therein), Holliger & Winter, Proc. Natl. Acad. Sci. USA 90, 6444-6448 (1993); Pei et al., Proc. Natl. Acad. Sci. USA 94, 9637-9642 (1997); Iliades et al., FEBS Lett. 409, 437-441 (1997); De Genst et al., J. Biol. Chem. 280, 14114-14121 (2005); De Genst et al., J. Biol. Chem. 279, 53593-53601 (2004); Dooley & Flajnik, Eur. J. Immunol. 35, 936-945 (2005); Streltsov & Nuttall, Immunol. Lett. 97, 159-160 (2005); Streltsov et al., Proc. Natl. Acad. Sci. USA 101, 12444-12449 (2004); Cortez-Retamozo et al., Cancer Res. 64, 2853-2857 (2004); Dottorini et al., Biochemistry 43, 622-628 (2004); Colby et al., J. Mol. Biol. 342, 901-912 (2004); Jespers et al., J. Mol. Biol. 337, 893-903 (2004); Linsley, Nat. Immunol. 6, 231-232 (2005); 37. Casey et al., Br. J. Cancer 86, 1401-1410 (2002); Weir et al., Biochem. Soc. Trans. 30, 512-516 (2002); Dolezal et al., Protein Eng. 16, 47-56 (2003); Power et al., Methods Mol. Biol. 207, 335-350 (2003); Arndt et al., FEBS Lett. 578, 257-261 (2004); Griffiths et al., J. Nucl. Med. 45, 30-39 (2004); Olafsen et al., Protein Eng. Des. Sel. 17, 21-27 (2004); Wittel et al., Nucl. Med. Biol. 32, 157-164 (2005); Le Gall et al., Protein Eng. Des. Sel. 17, 357-366 (2004); Kenanova et al., Cancer Res. 65, 622-631 (2005); Adams et al., Cancer Res. 64, 6200-6206 (2004); Grosse-Hovest et al., Int. J. Cancer; published online 7 Jul. 2005 (interscience.wiley.com/cgi-bin/abstract/110559371/ABSTRACT 120); Holliger et al., Cancer Res. 59, 2909-2916 (1999); Pattersen et al., J. Comput. Chem. 25, 1605-1612 (2004); Olafsen et al., Cancer Res. 65, 5907-5916 (2005); Shen et al., J. Nucl. Med. 46, 642-651 (2005); Nellis et al., Biotechnol. Prog. 21, 221-232 (2005); Ebbinghaus et al., Int. J. Cancer 116, 304-313 (2005); Wong et al., Clin. Cancer Res. 10, 5014-5021 (2004); Hulstein et al., Blood; published online 12 Jul. 2005 (bloodjournal.org/cgi/reprint/2005-03-1153v1)).

The term polyclonal and monoclonal antibodies, includes intact antibodies and functional (e.g., antigen-binding) antibody fragments, including Fab fragments, F(ab′)2 fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, and tetrabodies, tandem di-scFv, tandem tri-scFv. The term also encompasses intact or full-length antibodies, including antibodies of any class or subclass, including IgG and sub classes thereof, IgM, IgE, IgA, and IgD. Thus, although typically discussed in the context of IgG, the target antibody of the Ig-Fc-specific immunoglobulin variable domain can be IgM, IgE, IgA, or IgD.

In some forms, the antibodies are heterodimeric bi- and tri-(or more) specific Ig antibodies and Fc fusion proteins. Exemplary structures include, but are not limited to, IgG, IgM, mono-, di-, tri-, or more scFv-Fcs. For example, bispecific, trispecific, and multispecific formats include, but are not limited to, bispecific and trispecific IgG, IgG-scFv, IgG-dAb, scFv-Fc-scFv, knob-in-hole (KIH)-IgG, κλ-body, KIH0Fc-Fab/scFv, tandem scFv, KIH trispecific, bispecific Fc fusion (N- or C-terminal, with or without KIH).

In some forms, multispecific antibody molecules can include more than one antigen-binding site, where different sites are specific for different antigens. In some forms, multispecific antibody molecules can bind more than one (e.g., two or more) epitopes on the same antigen. In some forms, multispecific antibody molecules include an antigen-binding site specific for a target cell (e.g., cancer cell) and a different antigen-binding site specific for HERV-K Env.

In some forms, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

The disclosure encompasses chimeric antibodies and hybrid antibodies, with dual or multiple antigen or epitope specificities, and fragments, such as F(ab′)2 and the like, including hybrid fragments. Such antibodies and fragments can be made by techniques known in the art and can be screened for specificity and activity according to general methods for producing antibodies and screening antibodies for specificity and activity (see, e.g., Harlow and Lane. Antibodies, A Laboratory Manual. Cold Spring Harbor Publications, New York, (1988), which is hereby incorporated by reference).

The disclosure also encompasses human antibodies and/or humanized antibodies. Many non-human antibodies (e.g., those derived from mice, rats, or rabbits) are naturally antigenic in humans and, thus, can give rise to undesirable immune responses when administered to humans. Therefore, the use of human or humanized antibodies in the methods described herein serves to lessen the chance that an antibody administered to a human will evoke an undesirable immune response.

Human, chimeric, or humanized derivatives of the B5 antibody are particularly preferred for in vivo use in humans, however, murine antibodies or antibodies of other species may be advantageously employed for many uses (for example, in vitro or in situ detection assays, acute in vivo use, etc.). A humanized antibody can comprise amino acid residue substitutions, deletions or additions in one or more non-human CDRs. The humanized antibody derivative may have substantially the same binding, stronger binding or weaker binding when compared to a non-derivative humanized antibody. In specific forms, one, two, three, four, or five amino acid residues of the CDR have been substituted, deleted or added (i.e., mutated). Completely human antibodies are particularly desirable for therapeutic treatment of human subjects.

Human antibodies can be made by a variety of methods known in the art including phage display methods described above using antibody libraries derived from human immunoglobulin sequences (see U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741). Human antibodies can be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., purified B. mallei LPS. Monoclonal antibodies directed against the antigen can be obtained from the immunized, transgenic mice using conventional hybridoma technology (see, e.g., U.S. Pat. No. 5,916,771 and Yokoyama WM. Production of monoclonal antibody supernatant and ascites fluid. Curr Protoc Mol Biol Chapter 11: Unit 11.10; 2008). The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, CA) and Bristol Myers Squibb (New York, NY) can be engaged to provide human antibodies directed against a selected antigen using technology similar to that described above.

A humanized or chimeric version of the B5 antibody can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Preferably, the antibody also includes at least a portion of an immunoglobulin constant region (Fc), typically that of a human immunoglobulin. The constant domains of the antibodies may be selected with respect to the proposed function of the antibody, in particular the effector function which may be required. In some forms, the constant domains of the antibodies are (or comprise) human IgA, IgD, IgE, IgG or IgM domains. In a specific form, human IgG constant domains, especially of the IgG1 and IgG3 isotypes are used, when the humanized antibodies is intended for therapeutic uses and antibody effector functions such as antibody-dependent cell-mediated cytotoxicity (ADCC) and complement-dependent cytotoxicity (CDC) activity are needed. In alternative forms, IgG2 and IgG4 isotypes are used when the antibody is intended for therapeutic purposes and antibody effector function is not required. Disclosed are also antibodies with the Fc constant domains comprising one or more amino acid modifications which alter antibody effector functions such as those disclosed in U.S. Patent Application Publication Nos. 2005/0037000 and 2005/0064514.

In some forms, the antibody contains both the light chain as well as at least the variable domain of a heavy chain. In other forms, the antibody may further include one or more of the CH1, hinge, CH2, CH3, and CH4 regions of the heavy chain. The antibody can be selected from any class of immunoglobulins, including IgM, IgG, IgD, IgA and IgE, and any isotype, including IgG1, IgG2, IgG3 and IgG4. In some forms, the constant domain is a complement fixing constant domain where it is desired that the antibody exhibit cytotoxic activity, and the class is typically IgG1. In other forms, where such cytotoxic activity is not desirable, the constant domain may be of the IgG2 class. The antibody may contain sequences from more than one class or isotype, and selecting particular constant domains to evaluate desired effector functions is within the ordinary skill in the art.

The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including, but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592, 106 and EP 519,596; Padlan, 1991, Molecular Immunology 28(4/5):489-498; Studnicka et al., 1994, Protein Engineering 7(6):805-814; and Roguska et al., 1994, Proc. Natl. Acad. Sci. 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp): 5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

Antibodies as first and/or second binding agents are typically used in the disclosed kits and methods to form immunocomplexes to facilitate detection of the target analyte. In some forms, the immunocomplex is a “sandwich” immunocomplex, which includes a first antibody and a second antibody binding to the analyte, the analyte sandwiched between the first and second antibodies. In some forms, when the analyte is biotinylated and directly binds to the modified particles, only the analyte and a second antibody participate in formation of the immunocomplex.

In some forms, the antibody/antigen used in the present invention is described in the following Table 1.

TABLE 1
Antibody/Antigen pairs (including vendors and catalog numbers)

| Target | DAB (DNA barcoded) | Vendor | CAB (Biotinylated) | Vendor | Antigen | Vendor |
|---|---|---|---|---|---|---|
| IFNg | 502402 | BioLegend | M701B | ThermoFisher | CYT-206 | Prospec |
| IL-12p70 | M122 | ThermoFisher | M121B | ThermoFisher | 219-IL-025/CF | R&D |
| IL-1b | MAB601 | R&D | BAF201 | R&D | 200-01B | ThermoFisher |
| IL-6 | 501102 | BioLegend | 501202 | BioLegend | 206-IL-050 | R&D |
| TNFa | 502802 | BioLegend | 502904 | BioLegend | 210-TA | R&D |

### (B) Other Binding Agents

In some forms, other first and the second binding agent includes an aptamer, such as DNA, RNA or peptide aptamers. Nucleic acid aptamers are short single-stranded DNA or RNA oligos that are capable of binding a ligand (protein, small molecule, or even living cells) with high affinity due to their secondary structure. Most DNA or RNA is capable of forming a secondary structure, however only very rare sequences are capable of binding to a specific target with appreciable affinity. Aptamers, in addition to binding with high affinity, also bind with high specificity, as shown for an aptamer selected to bind theophylline. Aptamers are sometimes referred to as artificial antibodies, but aptamers have several advantages over antibodies, including ease and low cost of production which does not involve animals. Aptamers are less immunogenic than antibodies and are already being used as a therapeutic for humans.

Aptamers are obtained by rigorous selection, in which aptamers are “evolved” from pools of random DNA or RNA or amino acids, leaving few (if any) sequences capable of binding the target out of a high number of starting sequences. The random library is typically flanked by fixed primer regions such that each oligo in the pool contains the sequence 5′-primer1-N20-60-primer2 (reverse complement)-3′, where N is a random base. The primers are used to amplify the library after selection by PCR. The process to generate aptamers by in vitro selection was developed by the Szostak and Gold groups independently in 1990 and the process has become known as systematic evolution of ligands by exponential enrichment (SELEX). The SELEX procedure involves the use of the random library of DNA/RNA sequences being incubated with the target, followed by a partitioning step to remove unbound sequences, then followed by an elution step to recover the binding sequences, and then an amplification step to generate a library of sequences enriched for binding. Over the years, several variants of SELEX have arisen. One variant of SELEX using capillary electrophoresis (CE) allows for SELEX to be performed in a much shorter amount of time due to much more efficient partitioning and the prevention of aptamers binding to the ligand support (the ligand flows freely in buffer). In as little as one round of selection, and almost always less than five, strong binding highly specific aptamers may be selected, as opposed to traditional SELEX which typically takes 10 or more rounds of selection. CE-SELEX generated aptamers can have nM and even pM level disassociation constants.

d. First Reagent without First Binding Agent

In some forms, the first reagent does not include a first binding agent. When a first binding agent is not included, the modified magnetic particles bind to the target analyte directly through interaction between members of an affinity binding pair. In these forms, the magnetic particles are typically modified with a member of an affinity binding pair and the analyte is labeled with the complementary member of the binding pair, where the member and the complementary member can bind to each other via any suitable interactions such as those described above.

The affinity binding pairs used to bind the analyte to the magnetic particles can be any of the affinity binding pairs described above. For example, the affinity binding pair can be biotin or biotin derivatives (e.g., 2-iminobiotin), and biotin-binding compounds (e.g., avidin, streptavidin, neutravidin). Other examples of binding pairs can be glutathione and glutathione s-transferase, maltose and maltose-binding protein, and intein or chitin and chitin-binding protein.

Other suitable binding pairs include, receptor/ligand pairs, antibodies/antigens, natural or synthetic receptor/ligand pairs, hapten/antibody pairs, antigen/antibody pairs, epitope/antibody pairs, mimitope/antibody pairs, aptamer/target molecule pairs, hybridization partners, intercalater/target molecule pairs, and the use of a surface and anchoring reagent bound by electrostatic charge.

For the biotin-avidin binding pair such as biotin-streptavidin or biotin-NeutrAvidin pair, biotin can be attached to the analyte and streptavidin, avidin or NeutrAvidin may be attached to the carrier, surface, or particle, or vice versa. Biotin binds via affinity interactions to biotin-binding compounds, thereby non-covalently conjugating the analyte to the carrier, surface, or particle. In some forms, other affinity pairs used for immobilizing the analyte to a solid surface or particle include but are not limited to His-tag/Ni²⁺ or Co²⁺, Strep-tag/Strep-Tactin®, Protein A/IgG, Protein G/IgG, SBP-tag/Streptavidin, FLAG-tag/Anti-FLAG antibody resin, HA-tag/Anti-HA antibody resin, Myc-tag/Anti-Myc antibody resin, Halo-tag/HaloTag ligand, SpyTag/SpyCatcher.

ii. Characteristics

In some forms, the modified magnetic particle disclosed herein has a diameter of about 0.5 μm to about 5 μm, 0.3 μm-5.5 μm, 0.2 μm-6 μm, 1 μm-4.5 μm, 1.5 μm-4 μm, 2 μm-3.5, um, or 2.5 μm-3 μm.

In some forms, the modified magnetic particle disclosed herein a density of about 1.4 g DS/cm³-1.8 g DS/cm³, 1.3 g DS/cm³-1.9 g DS/cm³, 1.5 g DS/cm³-1.7 g DS/cm³, 1.2 g DS/cm³-2.0 g DS/cm³, 1.6 g DS/cm³-1.9 g DS/cm³, 1.1 g DS/cm³-2.1 g DS/cm³, or 1.0 g DS/cm³-2.5 g DS/cm³.

In some forms, the modified magnetic particles disclosed herein are uniform, non-porous, superparamagnetic, monodispersed and highly cross-linked polystyrene microspheres consisting of an even dispersion of magnetic material throughout the bead. The magnetic material within the magnetic particle consists of a mixture of maghemite (gamma-Fe2O3) and magnetite (Fe3O4). In some forms, the magnetic particles are coated with a thin polystyrene shell which encases the magnetic material and prevents any leakage from the beads or trapping of ligands in the bead interior. The shell also protects the target from exposure to iron while providing a defined surface area for the adsorption or coupling of various molecules. In some forms, the magnetic particles are surface activated with tosyl, epoxy, carboxylic acid and amine groups.

### 2. Second Reagent Including Second Binding Agent

The second reagent includes a second binding agent specific for one or each of a plurality of analytes of interest, conjugated to a nucleic acid barcode optionally, via a linker.

i. Linker

In some forms, a member of an affinity binding pair is linked to the magnetic particle via a linker. In some forms, the second binding agent is conjugated to a nucleic acid barcode via a linker. In some forms, the linker is formed by reaction between a crosslinking agent and a suitable functional group (such as amino group or azide group) of the second binding agent and the nucleic acid barcode. In some forms, the crosslinking agent is selected from a group consisting of but not limited to sulfosuccinimidyl 4-(N-maleimidomethyl)cyclohexane-1-carboxylate (Sulfo-SMCC), bis(sulfosuccinimide) suberate (BS3), Disuccinimidyl Suberate (DSS), Disuccinimidyl Sulfoxide (DSSO), and Disuccinimidyl Dibutyric Urea (DSBU). In preferred forms, the linker is bis(sulfosuccinimide) suberate (BS3).

In some forms, the linker is

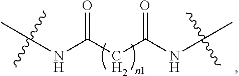

wherein n1 is an integer from 1 to 10, from 1 to 8, from 1 to 6, from 2 to 10, from 2 to 8, from 4 to 10, or from 4 to 8, such as 6; and wherein the secondary amino groups of the linker are from the second binding agent and the nucleic acid barcode respectively.

### BS3

BS3 is an amine-to-amine crosslinker that is homobifunctional, water-soluble, non-cleavable and membrane impermeable. BS3 contains an amine-reactive N-hydroxysulfosuccinimide (NHS) ester at each end of an 8-carbon spacer arm. NHS esters react with primary amines at pH 7-9 to form stable amide bonds, along with release of the N-hydroxysulfosuccinimide leaving group. Proteins, including antibodies, generally have several primary amines in the side chain of lysine (K) residues and the N-terminus of each polypeptide that are available as targets for NHS-ester crosslinking reagents.

### Sulfo-SMCC

Sulfosuccinimidyl-4-(N-maleimidomethyl)cyclohexane-1-carboxylate (Sulfo-SMCC) is a non-cleavable and membrane impermeable crosslinker. It contains an amine-reactive N-hydroxysuccinimide (NHS ester) and a sulfhydryl-reactive maleimide group. NHS esters react with primary amines at pH 7-9 to form stable amide bonds. Maleimides react with sulfhydryl groups at pH 6.5-7.5 to form stable thioether bonds. The maleimide groups of Sulfo-SMCC and SMCC are unusually stable up to pH 7.5 because of the cyclohexane bridge in the spacer arm. Because it contains the hydrophilic sulfonyl moiety, Sulfo-SMCC is soluble up to ~10 mM in water and many commonly used buffers, thus avoiding the use of organic solvents which may perturb protein structure.

### DBCO-Azide

Dibenzocyclooctyne (DBCO) reagent is a class of click chemistry labeling reagents. DBCO group can exclusively react with azide-tagged molecules or biomolecules to form a stable triazole. The click chemistry is also known as strain promoted alkyneazide cycloaddition (SPAAC), DBCO reagent has become widely used in bioconjugation, labeling and chemical biology. DBCO click chemistry can be run in aqueous buffer or in organic solvents depending on the property of the substrate molecules. Reagents with PEG arm will increases the compound's hydrophilicity.

### DSS

DSS is noncleavable and membrane permeable crosslinker that contains an amine-reactive N-hydroxysuccinimide (NHS) ester at each end of an 8-carbon spacer arm. NHS esters react with primary amines at pH 7-9 to form stable amide bonds, along with release of the N-hydroxysuccinimide leaving group. Proteins, including antibodies, generally have several primary amines in the side chain of lysine (K) residues and the N-terminus of each polypeptide that are available as targets for NHS-ester crosslinking reagents. DSS is first dissolved in an organic solvent such as DMF or DMSO, then added to the aqueous crosslinking reaction. BS3, the water soluble analog of DSS is also available for applications that require a hydrophilic crosslinker (e.g. to effect cell-surface crosslinking).

ii. DNA Barcode

In some forms, the second binding agent is conjugated to one or more components that act as a barcode or label. In some forms, these barcodes are nucleic acid barcodes. Barcodes and/or labels can be used to identify, isolate, sort, organize, degrade, maintain, store, purify or otherwise characterize or manipulate the biopolymer, or pool of biopolymers to which they are associated. Barcodes and labels can be selected from a wide variety of detectable, sortable or otherwise scorable molecules. Exemplary barcodes and labels include sequence identifiers, such as nucleotide or amino acid sequences; capture tags; and dyes or other detectable molecules. In some forms, one biopolymer includes one or more barcode or label. Barcodes or labels that can be used to capture the barcoded biopolymer for a pool of similar biopolymers are provided. Barcodes or labels that can be used to detect, quantify or otherwise assay the presence or absence of the biopolymer are provided. Barcodes or labels that facilitate the sorting or manipulation of the associated biopolymers are also provided. In some forms, the barcodes permit sorting, selecting, ordering, degradation, synthesis and manipulation of the associate biopolymers using microfluidic systems.

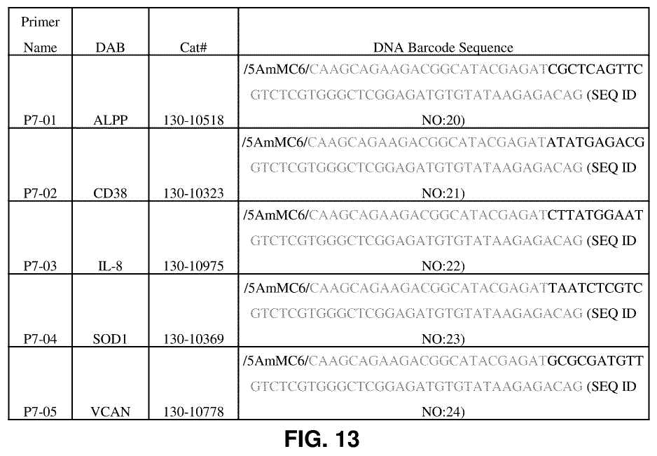

In some forms, the kit is used for detection of more than one type of analyte in one sample, such as more than 10 types of analytes, more than 100 types of analytes, or more than 1000 types of analytes in one sample. For example, the kit is used for detection of thousands of different proteins in one biological sample, such as blood. In these forms, the second binding agent specific for each type of analyte is labeled with a unique nucleic acid barcode. Each unique nucleic acid barcode corresponds to a single type of analyte. The nucleic acid barcodes for different types of analytes are distinguishable from one another. The PCR and/or next generation sequencing results of each unique nucleic acid barcode are thus indicative of the presence/absence and quantity of the specific type of analyte to which it corresponds. The nucleic acid barcodes can be created by designing unique nucleotide sequences. Exemplary nucleic acid barcodes are shown in Table 2 below and FIG. 13.

An exemplary barcoded biopolymer is a nucleic acid encoding bitstream data including a nucleotide sequence that acts as a barcode to identify the encoded data. A DNA barcode is a short DNA sequence that uniquely identifies a certain linked feature, such as nucleic acid sequence encoding one or more genes, or pieces of metadata. Linking features to DNA barcodes of homogenous length and melting temperature (Tm) allows experiments to be performed on the features in a pooled format, with subsequent deconvolution by PCR followed by microarray hybridization or high throughput sequencing. DNA barcode technology greatly improves the throughput of genetic screens, making possible experiments that would otherwise be quite time-consuming or laborious. Numerous resources and software tools are currently available for designing DNA microarray barcodes/probes (see, for example, Nielsen et al. Nucleic Acids Res 31:3491-3496 (2003); Rouillard, et al., Nucleic Acids Res 31:3057-3062 (2003); Wang, et al., Bioinformatics 19:796-802 (2003); Hu, et al. BMC Bioinformatics 8:350 (2007); and Markham et al., Methods Mol Biol 453:3-31 (2008)). DNA barcodes linked to genetic features greatly facilitate screening these features in pooled formats using microarray hybridization. Compositions of nucleic acid barcodes having distinct and detectable properties are known in the art. Xu et al describe the generation and characterization of 240,000 barcode probes, and test their performance by hybridization. Test hybridizations identified new probe design rules that significantly reduce cross-hybridization after their introduction into the framework of the algorithm. These rules should improve the performance of DNA microarray probe designs for many applications (Xu, et al., Proc Natl Acad Sci, 106 (7) 2289-2294 (2009)). Therefore, the described methods for microfluidic-based synthesis of biopolymers can produce barcoded nucleic acids including one or more barcodes that can be used to select a distinct biopolymer, or pool of biopolymers, based upon one or more of the sequence characteristics of the barcode. Exemplary characteristics that can be sued for the selection and isolation include thermal hybridization and melting temperature. The application of melting temperature to select and isolate a pool of biopolymers based upon melting and hybridization characteristics is represented in the Examples.

In some forms, sequence identifiers (i.e., barcodes) are included within initiator sequences. In other forms, the identifiers are attached to the initiator or to the growing biopolymer during the synthesis. In an exemplary form, a sequence identifier is attached to an initiator, or to a growing biopolymer as a single, pre-assembled unit.

Molecular or sequence barcoding is a method of identifying molecules from within a pool of other molecules. Barcoding is used for sequencing identification in next generation sequencing with complex pools of DNA strands. Barcoding can also be implemented for cell-based identification and RNA identification in solutions where parsing the sequences and samples are important for downstream separation of the samples. The synthesis of the DNA for barcoding is typically achieved by pre-synthesis of the sequence using methods known in the art and then ligated to the sample of interest by DNA ligase.

Exemplary DNA barcodes for specific analytes are shown in Table 2 and FIG. 13.

TABLE 2
Example DNA barcodes.

| Barcode name | DNA Barcode Sequence |
|---|---|
| Bp1 | AGCACACATTGCTGGTTCTGCTCAC |
| | GTCTGACTACAGGCATTCCGTCTGT |
| | CTCTTATACACATCTCCGAGCCCAC |
| | GAGAC |
| | (SEQ ID NO: 1) |
| Bp3 | AGCACACATTGCTGGTTCTGCTCAC |
| | GTCAACCTAGTGCGACGATACCTGT |
| | CTCTTATACACATCTCCGAGCCCAC |
| | GAGAC |
| | (SEQ ID NO: 2) |
| Bp5 | AGCACACATTGCTGGTTCTGCTCAC |
| | GCCGAGGTCTGTCACAGTGTACTGT |
| | CTCTTATACACATCTCCGAGCCCAC |
| | GAGAC |
| | (SEQ ID NO: 3) |
| Bp7 | AGCACACATTGCTGGTTCTGCTCAC |
| | GGTGAACCGTATCTGACGGCACTGT |
| | CTCTTATACACATCTCCGAGCCCAC |
| | GAGAC |
| | (SEQ ID NO: 4) |
| Bp8 | AGCACACATTGCTGGTTCTGCTCAC |
| | GTATCGCCCAAGAGCGCAGTCCTGT |
| | CTCTTATACACATCTCCGAGCCCAC |
| | GAGAC |
| | (SEQ ID NO: 34) |
| Bp9 | AGCACACATTGCTGGTTCTGCTCAC |
| | GTCCCGTTGTATTAGCCGCCGCTGT |
| | CTCTTATACACATCTCCGAGCCCAC |
| | GAGAC |
| | (SEQ ID NO: 5) |
| Bp10 | AGCACACATTGCTGGTTCTGCTCAC |
| | GACGCAGGATCTAAGCCAGCGCTGT |
| | CTCTTATACACATCTCCGAGCCCAC |
| | GAGAC |
| | (SEQ ID NO: 35) |

### 3. Additional Components

i. Buffers and Reagents

In some forms, a kit for detection of a target analyte in a sample includes buffers. Various buffer solutions are utilized throughout the BIQ-ELISA process to evaluate different steps. A coupling buffer, including 200 mM HEPES at pH 8.5, is used for preparing oligonucleotide solutions and facilitating the conjugation of oligonucleotides with antibodies during the DAB-ONA preparation. For purification via Fast Protein Liquid Chromatography (FPLC), two buffers are used: Buffer A, including 150 mM NaCl and 50 mM Tris-HCl at pH 8.0, and Buffer B, including 1 M NaCl and 50 mM Tris-HCl at pH 8.0. These buffers allow efficient elution and purification of antibody-oligonucleotide conjugates. The dilution buffer include PBS containing 0.05% Tween-20 (PBST) or variations thereof. Wash buffers, important for removing unbound components, include PBST (PBS with 0.05% Tween-20) and PBS with 0.1% Tween-20. Elution buffers such as 10 mM NaOH are used to release DNA from magnetic beads; this concentration maintains both elution efficiency and compatibility with subsequent qPCR reactions. Additionally, qPCR reaction buffers are prepared using a 2×PCR premix containing primers, probes, and SYBR Green dye for fluorescence-based detection. These buffers are carefully formulated to ensure compatibility with eluted DNA and minimize background from primer-dimers. Each buffer is specifically tailored to enhance the sensitivity, specificity, and reliability of the BIQ-ELISA system.

A buffer can be an aqueous solution that provides optimal pH, ionic strength, cofactors, and the like for optimal enzyme activity. In some forms, the buffers are suitable for storage of the enzymes. In some forms, the buffers are suitable for PCR. Suitable buffer components include, without limitation, one or more salts, reducing agents (e.g., Dithiothreitol), buffering agents, deoxynucleoside triphosphates (dNTPs), or combinations thereof. The one or more salts provide monovalent or divalent cations, such as, Mg2+, Mn2+, K+, NH4+, and Na+. Exemplary salts that can be included in the buffers are KCl, MgCl2, NaCl, MnCl2, NH4Cl, MgSO4, (NH4)2SO4, and magnesium acetate. The concentration of the one or more salts can be in the range of from about 1 mM to about 500 mM, about 5 mM to about 250 mM, about 10 mM to about 200 mM, about 25 mM to about 150 mM, or about 50 mM to about 100 mM.

Suitable buffering agents are known in the art and include, without limitation, tris (e.g., Tris-HCl), tricine, bicine, and HEPES. The buffering agent can have a pH in the range of about 6 to 10 (e.g., a pH of 6.8 to 9, such as about pH 8.5). The concentration of the one or buffering agents can be in the range of from about 10 mM to about 100 mM.

In some forms, the reagents used in the BIQ-ELISA kit include a PCR master mix, which contains DNA polymerase and nucleotides necessary for qPCR amplification, providing the enzymatic activity and building blocks for DNA synthesis. Additionally, one or more oligonucleotide primers are used to specifically amplify the ONA barcode, ensuring accurate and efficient target amplification. A TaqMan probe labeled with a fluorescent reporter and quencher is employed for detecting the amplified ONA barcode, facilitating precise quantification through fluorescence signals during qPCR. These reagents are integral to achieving the sensitivity and specificity required for the BIQ-ELISA system.

Any buffer(s) and reagent(s) used in the BIQ-ELISA system, such as those described above, can also be used in the BIQL assay. The BIQL assay can further include additional buffer(s) and reagents(s) for protein labeling to introduce a member of an affinity pair on the protein. When protein labeling is included in the method, the sample, containing the analytes, is incubated in a buffer with the protein labeling reagent. Any buffer suitable for facilitating the reaction between the proteins in the sample and the protein labeling reagent can be used in this step. For example, suitable protein labeling reagent, such as any of those described in the “Protein Labeling Reagent” section below, in Phosphate Buffered Saline (PBS) ranging from pH 7 to pH 8 can be used during this labeling reaction.

ii. Blocking Agents

In some forms, blocking agents are utilized to reduce background noise. When a blocking agent is used, the blocking agent can be included in a buffer solution, such as a blocking buffer containing 10% casein, 5% BSA, and Biolipidure®-1002 (“B1002”). Buffer solution containing blocking agent(s) can be used in the assay in any way as desired. For example, buffer solutions containing blocking agent(s) are used to dilute the samples, to prepare the bead and conjugate reagents, and/or in the first step (e.g., incubation of the sample or biotinylated sample with the first and second reagents) of the immunoassay.

Several blocking agents can be used in the BIQ-ELISA process to reduce background noise and enhance assay sensitivity. One such agent is PBS containing 10% casein and 5% bovine serum albumin (BSA), referred to as 10C5B, which was tested for its blocking efficiency. Another formulation, CB1002, contains PBS with 10% casein and 1% (w/v) B1002, offering a lower BSA content as an alternative to 10C5B. Additionally, a blocking solution containing PBS with 5% BSA and 0.1% Tween-20 was utilized, though it exhibited higher background signals in some experiments. A Biolipidure mix was also tested, prepared by combining 50 μL of B203, B206, B802, B804, and B1002, each as 5% (w/v) solutions in 4750 μL PBS, to explore its effectiveness in minimizing background interference. These formulations were evaluated to select the blocking agent for the assay. Such blocking agents can also be used in the BIQL assay.

In some forms, blocking buffers are used to prevent or reduce the activity of a catalyst, for example, a polymerase enzyme. In some forms, the stop or block reagent quenches the enzymic catalysis that incorporates the component building block onto the growing biopolymer chain. Typically, the methods include stop reagents and/or blocking reagents that are specific or effective to stop, reduce or otherwise mediate the activity of the catalyst enzyme that is employed. Blocking buffers and stop reagents effective for specific catalyst enzymes are known in the art.

iii. Protein Labeling Reagent

In some forms, when the analytes of interest directly bind to particles modified with a member of an affinity binding pair, the disclosed kits contain a protein labeling reagent for introducing a complementary member of the affinity binding pair to the analytes in the sample. In these forms, the protein labeling reagent universally labels all proteins in the sample—the specific detection of analytes of interest depends on the second binding agents and the corresponding nucleic acid barcodes. For example, all biotinylated proteins bind to the streptavidin on the modified magnetic particles; however, only proteins that are of interest (analytes) are recognized and bound by the second binding agents conjugated to nucleic acid barcodes, and the nucleic acid barcode unique to each type of analytes allows detection and distinction of the analytes.

The protein labeling reagent can non-covalently or covalently attach one or more molecules to a protein. This can be done either chemically or enzymatically. Typically, chemical labeling of proteins involves using a protein labeling reagent that contains a reactive moiety that is responsible for attachment to the protein and the label molecule. In some forms, the reactive moiety of the protein labeling reagent is an amine-reactive N-hydroxysulfosuccinimide (NHS) ester. NHS esters react with primary amines at pH 7-9 to form stable amide bonds, along with release of the N-hydroxysulfosuccinimide leaving group. Proteins generally have several primary amines in the side chain of lysine (K) residues and the N-terminus of each polypeptide that are available as targets for NHS-ester crosslinking reagents. Other examples of reactive moieties that can be used in the kits for protein labeling are maleimide (e.g., reactive with sulfhydryls), primary amines in combination with carbodiimides (e.g., reactive with carboxyls), and hydrazines and alkoxyamines (e.g., reactive with glycoproteins), and any combination thereof. In some forms, the protein labeling reagent also contains a linker between the reactive moiety and the member of an affinity binding pair (such as biotin, 2-iminobiotin, glutathione, maltose, or an aptamer). Polyethylene glycol (PEG) is commonly used as a linker to increase the solubility of the protein labeling reagent.

In some forms, the protein labeling reagent is a biotinylation reagent, which contains biotin or a biotin derivative; an amine-reactive moiety, such as an NHS ester; and optionally a linker between the reactive moiety and the biotin or biotin derivative. For example, biotinylation reagent can contain a (PEG) n, wherein n is 1, 2, 3, 4, 5, 6, 7, 8, 9, 10 or more repeating ethylene glycol subunits linking the reactive moiety and the biotin or biotin derivative. In an exemplary kit, the biotinylation reagent is EZ-Link NHS-PEG4-Biotin that is composed of an NHS ester as the reactive moiety, (PEG) 4 as the linker, and biotin as the label moiety.

### C. Methods of Use

Methods of using the disclosed kit for detecting one or more analytes in a sample are provided.

The method generally includes forming an immunocomplex in the presence of the analyte to be detected and amplifying the DNA barcode on the second binding partner. In some forms, the procedure takes a total of up to four hours for completion (e.g., from sample preparation or sample incubation to the completion of NGS detection). For example, the procedure takes from about 30 min to about 4 hours, from about 30 min to about 3 hours, from about 30 min to about 2 hours, from about 30 min to about 1 hour, from about 1 hour to about 2 hours, from about 1 hour to about 3 hours, from about 1 hour to about 4 hours, from about 2 hours to about 4 hours, from about 2 hours to about 3 hours, or from about 3 hours to about 4 hours for completion.

In some embodiments, the formation and purification of the sandwich immunocomplex (ONA-DAB-Antigen-CAB-Biotin-SA-Beads) is as follows. Samples are incubated with DAB-ONA and CAB-Biotin-SA-Beads in a supplied 96-well plate. In the presence of the target protein, the DAB-ONA binds to the target and attaches to the CAB-Biotin-SA-Beads via the biotin-streptavidin interaction and the unbound proteins are removed through washing with a magnetic automated washer.

In some other embodiments, the formation and purification of the immunocomplex formed between the analyte and the second binding agent (ONA-DAB-Analyte-Biotin-SA-Beads) is as follows. Proteins in a sample are universally biotinylated with a biotinylation reagent. Biotinylated proteins are then incubated with SA-Beads and DAB-ONA in a supplied 96-well plate. The biotinylated proteins bind to the SA-Beads. In the presence of a target analyte, the DAB-ONA binds to the analyte that is bound to the SA-Beads via biotin-streptavidin interaction and the unbound proteins are removed through washing with a magnetic automated washer.

In such systems, all proteins in the sample are first biotinylated, which resulted in universal labeling. These biotinylated proteins are immobilized onto streptavidin-coated magnetic beads and then incubated with an array of DNA-barcoded detection antibodies to form highly specific immunocomplexes. Unlike traditional sandwich assays that require a secondary antibody, this platform leverages direct quantification via qPCR or next-generation sequencing (NGS), using the DNA tags as unique molecular identifiers. BIQL integrates the femtogram-level sensitivity of bead-based immuno-qPCR (BIQ) with the scalable multiplexing power of biotin-labeled sample arrays. By combining direct biotinylation of proteins, magnetic bead capture, and DNA-barcoded antibody detection, BIQL facilitates digital, highly specific quantification of up to 1000+ proteins per run—ideal for clinical biomarker discovery, systems biology, and next-generation diagnostics.

The bound immunocomplex is released from the beads using a suitable elution buffer and transferred as the template for qPCR. Primers and PCR master mix are added to the wells and data will be collected using qPCR. Ct values obtained from the qPCR are then used to calculate the amount of antigen contained in each sample, where lower Ct values indicate a higher concentration of antigen. A set of target protein standards with known concentrations can be ran simultaneously to generate a standard curve for quantifying unknown samples.

### 1. Analytes to be Detected

The disclosed kit can be used to detect one or more analyte(s) (also referred to herein as analyte molecule(s)). In some forms, the methods and kits described herein can be used in an immunoassay to detect one or more of the following classes of biomarkers: cytokines, circulating tumor-specific proteins, proteins associated with one or more infectious diseases, intracellular markers, etc., and combinations thereof. In some forms, the analyte is IL-8 (Interleukin-8), ALPP (Alkaline phosphatase for example, placental type (EC 3.1.3.1)), CD38 (Cluster of differentiation 38), SOD1 (Superoxide dismutase-1), or VCAN (Versican), or a combination thereof. In some forms, the analyte is MIP-1α, Eotaxin, IL-22, IL-12p40, IL-12p70, EXN4, IFN-γ, IL-1β, IL-6, or TNF-α, or a combination thereof.

In some forms, the kits disclosed herein binds to target analytes that are homodimers, homotrimers, etc. and where each monomer of the dimer binds to the first binding agent, and a second monomer binds to the second binding agent.

The kit and methods of the present disclosure can be used to determine the concentration of one or more, e.g., two or more analytes in a sample. Thus, one or more analytes can be measured in the same sample. Panels of analytes that can be measured in the same sample include, for example, panels of assays for analytes or activities associated with a disease state or physiological conditions. Certain such panels include panels of cytokines and/or their receptors (e.g., one or more of TNF-alpha, TNF-beta, IL1-alpha, IL1-beta, IL2, IL4, IL6, IL-10, IL-12, IFN-y, etc.), growth factors and/or their receptors (e.g., one or more of EGF, VGF, TGF, VEGF, etc.), drugs of abuse, therapeutic drugs, vitamins, pathogen specific antibodies, auto-antibodies (e.g., one or more antibodies directed against the Sm, RNP, SS-A, SS-alpha, JO-1, and Sc1-70 antigens), allergen-specific antibodies, tumor markers (e.g., one or more of CEA, PSA, CA-125 II, CA 15-3, CA 19-9, CA 72-4, CYFRA 21-1, NSE, AFP, etc.), markers of cardiac disease including congestive heart disease and/or acute myocardial infarction (e.g., one or more of Troponin T, Troponin I, Troponin C, myoglobin, CKMB, myeloperoxidase, glutathione peroxidase, β-natriuretic protein (BNP), alpha-natriuretic protein (ANP), endothelin, aldosterone, C-reactive protein (CRP), etc.), markers associated with hemostasis (e.g., one or more of Fibrin monomer, D-dimer, thrombin-antithrombin complex, prothrombin fragments 1 and 2, anti-Factor Xa, etc.), markers of acute viral hepatitis infection (e.g., one or more of IgM antibody to hepatitis A virus, IgM antibody to hepatitis B core antigen, hepatitis B surface antigen, antibody to hepatitis C virus, etc.), markers of Alzheimers Disease (alpha-amyloid, beta-amyloid, Aβ 42, Aβ 40, Aβ 38, Aβ 39, Aβ 37, Aβ 34, tau-protein, etc.), markers of osteoporosis (e.g., one or more of cross-linked Nor C-telopeptides, total deoxypyridinoline, free deoxypyridinoline, osteocalcin, alkaline phosphatase, C-terminal propeptide of type I collagen, bone-specific alkaline phosphatase, etc.), markers of fertility state or fertility associated disorders (e.g., one or more of Estradiol, progesterone, follicle stimulating hormone (FSH), lutenizing hormone (LH), prolactin, hCG, testosterone, etc.), markers of thyroid disorders (e.g., one or more of thyroid stimulating hormone (TSH), Total T3, Free T3, Total T4, Free T4, and reverse T3), and markers of prostate cancer (e.g., one or more of total PSA, free PSA, complexed PSA, prostatic acid phosphatase, creatine kinase, etc.). Certain forms of disclosure include measuring, e.g., one or more, two or more, four or more or 10 or more analytes associated with a specific disease state or physiological condition (e.g., analytes grouped together in a panel, such as those listed above; e.g., a panel useful for the diagnosis of thyroid disorders can include e.g., one or more of thyroid stimulating hormone (TSH), Total T3, Free T3, Total T4, Free T4, and reverse T3).

In some forms, the panel includes one or more low abundance analytes in traditional sample matrices, e.g., analytes at a concentration of less than about 100 fg/mL, and preferably, less than about 10 fg/mL. A non-limiting list of analytes that can be included in the panel includes, e.g., IL-17, IL-21, IL-31, Ab-38, Ab-40, Ab-42, Ab-39, Ab-43, Ab-15, Ab-16, Ab-17, Abeta oligomers, C-peptide, IL-13, IL-17A, IL-2, IL-4, IL-5, IL-6, IL-8, IL-12/23p40, IL-12p70, INF-g, PSA, PSAc, Tau, phospho-Tau, TNFa, troponin I, cardiac troponin T, troponin C, VEGF, VEGF-A, VEGF-B, VEGF-C, VEGF-D, EPO, LC3B, albumin, CHO-P, E. coli HCP, IgA, IgE, IgG, IgG₁, IgG₄, IgM, NSO-P, Per-C6, residual protein A, IgG₂, IgG₃, IgG₄, AFP, CA125, Caspase-3 active, CXCL11/I-TAC, ErbB2/HER2, HGFR/o-MET, IFN-beta, MMP1, MMP2, MMP3, MMP9, beta-NGF, TFF3, TIMP1, Kim-1, alpha-2 macroglobulin, D-dimer, ICAM-1, myeloperoxidase, myoglobin, PAI-1, PCSK9, plasminogen, renin/prorenin, tPA, CXCL1/GRO-alpha, CCL2/MCP1, CCL3/MIP-1alpha, CCL4/MIP-1beta, CCL5/Rantes, CRP, CXCL9/MIG, CXCL10/IL-10, G-CSF, GM-CSF, IFN-alpha, IFN-gamma, IL1alpha, IL-1beta, IL2, IL3, IL4, IL5, IL6, IL7, IL8, IL12 (p70), IL13, IL15, IL18, IL-22, IL-23, IL-33, c-MET, adiponectin, FGF21, TSLP, GLP-1, growth hormone, IGF1, IGF2, insulin, leptin, prolactin, HIV p24, HB-EGF, AKT, phospho-AKT, and combinations thereof.

In some form, the kits and methods described herein can be used to detect analytes that are in low abundance due to a recent exposure and/or infection. Early diagnosis of various diseases or conditions, e.g., cancer, bacterial infections, e.g., Bacillus anthracis (Anthrax), viral infections, e.g., HIV, hepatitis, HPV, etc., toxin exposure, e.g., ricin, botulinum toxin A, B, or E, etc., is limited by the fact that the limits of detections (LOD) of available technologies, such as ELISA, are higher than the circulating concentrations of low abundance proteins that could indicate the onset of disease. The panel can include one or more low abundance analytes in traditional sample matrices, e.g., analytes at a concentration of less than about 100 fg/mL, or less than about 10 fg/mL. A non-limiting list of analytes that can be included in the panel includes, e.g., HIVgp41, HIVgp120, HIVgp160, HIVp24, HIVp66, HIVp51, HIVp17, HIVp31, Tat, Nef, Viv, hepatitis A, B, C, D, or E antigens, HPV types 16, 18, 31, 33, 35, 39, 45, 51, 52, 56, 58, 59, 68, 73, and/or 82, HPV-E6 and E7 proteins, IL-17, IL-21, IL-31, IL-22, IL-23, IL-33, cardiac troponin T, and combinations thereof. Still further, the panel can also include one or more of the follow analytes that can be in low abundance due to recent disease onset, exposure and/or infection: Ab-38, Ab-40, Ab-42, Ab-39, Ab-43, Ab-15, Ab-16, Ab-17, Abeta oligomers, C-peptide, IL-13, IL-17A, IL-2, IL-4, IL-5, IL-6, IL-8, INF-g, PSA, Tau, phospho-Tau, TNFa, troponin I, cardiac troponin T, troponin C, VEGF, VEGF-A, VEGF-B, VEGF-C, VEGF-D, EPO, LC3B, albumin, CHO-P, E. coli HCP, IgA, IgE, IgG, IgG1, IgG4, IgM, NSO-P, Per-C6, residual protein A, IgG2, IgG3, IgG4, AFP, CA125, Caspase-3 active, CXCL11/I-TAC, ErbB2/HER2, HGFR/o-MET, IFN-beta, MMP1, MMP2, MMP3, MMP9, beta-NGF, TFF3, TIMP1, Kim-1, alpha-2 macroglobulin, D-dimer, ICAM-1, myeloperoxidase, myoglobin, PAI-1, PCSK9, plasminogen, renin/prorenin, tPA, CXCL1/GRO-alpha, CCL2/MCP1, CCL3/MIP-1alpha, CCL4/MIP-1beta, CCL5/Rantes, CRP, CXCL9/MIG, CXCL10/IL-10, G-CSF, GM-CSF, IFN-alpha, IFN-gamma, IL1alpha, IL-1beta, IL-3, IL-7, IL-12 (p70), IL-13, IL-15, IL-18, c-MET, adiponectin, FGF21, GLP-1, growth hormone, IGF1, IGF2, insulin, leptin, prolactin, HB-EGF, AKT, phospho-AKT, and combinations thereof.

Examples of samples that may be analyzed by the present disclosure include, but are not limited to food samples (including food extracts, food homogenates, beverages, etc.), environmental samples (e.g., soil samples, environmental sludges, collected environmental aerosols, environmental wipes, water filtrates, etc.), industrial samples (e.g., starting materials, products or intermediates from an industrial production process), human clinical samples, veterinary samples and other samples of biological origin. Biological samples that can be analyzed include, but are not limited to, feces, mucosal swabs, physiological samples and/or samples containing suspensions of cells. Specific examples of biological samples include blood, serum, plasma, feces, mucosal swabs, tissue aspirates, tissue homogenates, cell cultures and cell culture supernatants (including cultures of eukaryotic and prokaryotic cells), urine, saliva, sputum, and cerebrospinal sample.

Analytes that can be measured using the kit of the disclosure include, but are not limited to proteins, toxins, nucleic acids, microorganisms, viruses, cells, fungi, spores, carbohydrates, lipids, glycoproteins, lipoproteins, polysaccharides, drugs, hormones, steroids, nutrients, metabolites and any modified derivative of the above molecules, or any complex including one or more of the above molecules or combinations thereof. The level of an analyte of interest in a sample can be indicative of a disease or disease condition, or it can simply indicate whether the patient was exposed to that analyte.

In some forms, the methods of the present disclosure allow detection of a wide variety of biological and biochemical agents, as described above. In one form, the methods can be used to detect pathogenic and/or potentially pathogenic virus, bacteria and toxins including biological warfare agents (“BWAs”) in a variety of relevant clinical and environmental matrices, including and without limitation, blood, sputum, stool, filters, swabs, etc. A non-limiting list of pathogens and toxins that can be analyzed (alone or in combination) using the methods of the present disclosure is Bacillus anthracis (anthrax), Yersinia pestis (plague), Vibrio cholerae (cholera), Francisella tularensis (tularemia), Brucella spp. (Brucellosis), Coxiella burnetii (Q fever), listeria, salmonella, shigella, V. cholera, Chlamydia trachomatis, Burkholderia pseudomallei, orthopox viruses including variola virus (smallpox), viral encephalitis, Venezuelan equine encephalitis virus (VEE), western equine encephalitis virus (WEE), eastern equine encephalitis virus (EEE), Alphavirus, viral hemorrhagic fevers, Arenaviridae, Bunyaviridae, Filoviridae, Flaviviridae, Ebola virus, staphylococcal enterotoxins, ricin, botulinum toxins (A, B, E), Clostridium botulinum, mycotoxin, Fusarium, Myrothecium, Cephalosporium, Trichoderma, Verticimonosporium, Stachybotrys, glanders, wheat fungus, Bacillus globigii, Serratia marcescens, yellow rain, trichothecene mycotoxins, Salmonella typhimurium, aflatoxin, Xenopsylla cheopis, Diamanus montanus, alastrim, monkeypox, Arenavirus, Hantavirus, Lassa fever, Argentine hemorrhagic fevers, Bolivian hemorrhagic fevers, Rift Valley fever virus, Crimean-Congo virus, Hanta virus, Marburg hemorrhagic fevers, yellow fever virus, dengue fever viruses, influenza (including human and animal strains including H5N1 avian influenza, influenza A, influenza A, H1 specific, influenza A, H3 specific, influenza A, H5 specific, influenza A, 2009-H1N1 specific, influenza B), RSV, human immunodeficiency viruses I and II (HIV I and II), hepatitis A, hepatitis B, hepatitis C, hepatitis (non-A, B or C), Enterovirus, Epstein-Barr virus, Cytomegalovirus, herpes simplex viruses, Chlamydia trachomatis, Neisseria gonorrheae, Trichomonas vaginalis, human papilloma virus, Treponema pallidum, Streptococcus pneumonia, Borellia burgdorferi, Haemophilus influenzae, Mycoplasma pneumoniae, Chlamydophila pneumoniae, Legionella pneumophila, Staphylococcus aureus, Staphylococcus Enterotoxin B (SEB), Abrin, Shiga Toxin 1, Shiga Toxin 2, Moraxella catarrhalis, Streptococcus pyogenes, Clostridium difficile, Neisseria meningitidis, Klebsiella pneumoniae, Mycobacterium tuberculosis, Group A Streptococcus, E. coli 0157, coronavirus, Coxsackie A virus, rhinovirus, parainfluenza virus, respiratory syncytial virus (RSV), metapneumovirus, vaccinia, and adenovirus.

### 2. Analyte Labeling

In some forms, the method does not require two binding agents for a sandwich type immunoassay; rather, the analytes directly bind to the particles via an affinity binding pair and a second binding agent binds to the surface bound analyte. In these forms, the method includes a step of labeling the analytes with a protein labeling reagent, such as any one of those described above. The protein labeling reagent universally labels all proteins in the sample—the specific detection of analytes of interest depends on the second binding agents and the corresponding nucleic acid barcodes. For example, all biotinylated proteins bind to the streptavidin on the modified magnetic particles; however, only proteins that are of interest (analytes) are recognized and bound by the second binding agents specific for the proteins of interest. The second binding agents are conjugated to nucleic acid barcodes, and the nucleic acid barcode unique to each type of analytes allows detection and distinction of the analytes. Following the labeling step, the sample containing labeled analytes (and all other labeled proteins) is incubated with the modified magnetic particles that contain a complementary member of the binding pair.

When analyte labeling is included in the method, the sample, containing the analytes, is incubated in a buffer with the protein labeling reagent. Any buffer suitable for facilitating the reaction between the target analyte and the protein labeling reagent can be used in this step. For example, Phosphate Buffered Saline (PBS) ranging form pH 7 to pH 8 can be used during this labeling reaction. During incubation, the analyte reacts with the protein labeling reagent to attach a member of an affinity binding pair to the analyte. The member introduced onto the analyte is complementary and binds with the member on the surface of the modified particles. This results in anchoring of the analytes on the surface of the modified magnetic particles without the need of a surface bound binding agent. For example, a biotinylation reagent attaches biotin onto the analyte, which in turn binds to the streptavidin on the surface of a modified magnetic particle.

### 3. Incubating Samples to Form an Immunocomplex

The disclosed method includes incubating the sample with the modified magnetic particles and the second binding agent to form an immunocomplex.

In some forms, the modified magnetic particles are pre-coated with a first binding agent such as biotinylated capture antibody (CAB) that is bound to a member of an affinity pair (such as streptavidin/biotin pair) on the particle surface. The sample, containing the target antigen, is incubated with these particles along with a second binding agent (e.g. detection antibody (DAB)) conjugated to an oligonucleotide (ONA) barcode. During incubation, the target antigen binds specifically to the first binding agent on the magnetic particles, while the second binding agent simultaneously binds to another epitope on the same antigen. This forms a sandwich-like immune complex (for e.g. CAB-antigen-DAB), where the biotin-streptavidin interaction secures the complex to the magnetic particles. This step ensures high specificity and efficient binding, forming the foundation for subsequent purification and detection.

In some forms, the method does not use two binding agents for a sandwich-like immunocomplex; rather, labeled proteins are directly bound to a member of an affinity pair (such as streptavidin/biotin pair) on the particle surface. In these forms, the target analyte is bound to the modified magnetic particle through the biotin-streptavidin interaction, and the second binding agent is the only binding agent participating in formation of the immunocomplex.

### 4. Washing and Eluting DNA Barcode

The washing process involves multiple steps to remove unbound and non-specifically bound molecules from the immunocomplex. After incubating the sample with magnetic beads and binding agents, the reaction plate is placed on a magnetic rack to stabilize the beads. The supernatant containing unbound components is carefully removed, and the wells are washed multiple times with a washing buffer such as PBS containing 0.1% Tween-20. This process may be automated using a magnetic auto-washer to ensure thorough cleaning and minimize background noise. Residual liquid is removed after the final wash step by centrifugation or drying.

To release the oligonucleotide (ONA) barcode from the magnetic beads, an elution buffer, typically, NaOH, is used. The solution is incubated at room temperature for 10 minutes to dissociate the barcode from the immune complex. The reaction plate is then returned to the magnetic rack to separate the beads, and the supernatant containing the eluted DNA barcode is carefully transferred to a clean PCR plate for subsequent amplification.

### 5. Performing Nucleotide Amplification or Sequencing

The disclosed method includes performing nucleotide amplification and/or sequencing of the nucleotide barcode.

i. PCR

The disclosed method includes performing nucleotide amplification such as quantitative PCR (qPCR) after the formation and purification of the immune complex disclosed above. For qPCR, the bound oligonucleotide (ONA) barcode, conjugated to the second binding agent (such as DAB), is released from the magnetic particles by elution with NaOH. The eluted oligonucleotides are transferred to a qPCR reaction mix containing a PCR master mix (including DNA polymerase and nucleotides), oligonucleotide primers specific to the ONA sequence, and a TaqMan probe labeled with a fluorescent reporter and quencher. During qPCR, the ONA barcode is amplified, and fluorescence signals are monitored in real-time, where lower Ct values correspond to higher antigen concentrations.

In some forms, qPCR is performed using a detection probe and a quencher oligonucleotide. Detection probe refers to a labelled oligonucleotide which can be used to inform the presence of a specific target nucleic acid. Typically, a detection probe can form a duplex structure with a sequence within the amplified target nucleic acid, due to complementarity of the probe with a sequence in the target region. In some forms, the detection probe includes a fluorophore, a first region containing the tag sequence (or a portion thereof) of the first PCR primer, and a second region containing a (target-specific) sequence that is complementary to the amplified target nucleic acid product. In some forms, the detection probe includes a fluorophore, and a second region containing a (target-specific) sequence that is complementary to the amplified target nucleic acid product. In some forms, the first region includes about 7-12 nucleotides and/or the second region includes about 15-25 nucleotides.

Generally, the melting temperature (Tₘ) of the detection probes is designed to be about 70-78° C., inclusive.

Generally, the annealing temperature (Tₐ) of the detection probes is designed to be between about 65-83° C., inclusive.

In some forms, the detection probe can be designed to hybridize/anneal to the target at a higher temperature (e.g., 5-10° C. higher) than which the forward and/or reverse primers anneal, in order to ensure detection before primers are extended. If the detection probe binds to the target at the same time or after the forward/reverse primers bind, the polymerase may begin replication of target that does not contain bound detection probe. As a result, new DNA will be synthesized without detecting fluorescence from a previous round. Such a situation can lead to inaccurate data.

In some forms, the detection probe is designed to bind/hybridize to the target of interest at 70° C. or more. In some forms, the detection probe is designed such that any detection probe not bound to target will be quenched at 58-62° C., inclusive (e.g., due to binding to the quencher oligonucleotide).

The quencher oligonucleotide is an oligonucleotide that can hybridize with the detection probe. In some forms, the quencher oligonucleotide includes a fluorescence quencher and a sequence that is fully or partially complementary to the first region of the detection probe. In some forms, the quencher oligonucleotide includes a fluorescence quencher and a sequence that is fully complementary to the detection probe. Typically, the quencher oligonucleotide contains about 10-12 nucleotides and/or the detection probe contains about 30 nucleotides or more (e.g., 30, 31, 32, 33, 34, 35, 36, 37, 38, 39, 40 nucleotides or more).

Generally, the melting temperature (Tₘ) of the quencher is designed to be about 24-62° C., inclusive.

Generally, the annealing temperature (Tₐ) of the quencher is designed to be about 19-65° C., inclusive.

Typically, the quencher oligonucleotide and the detection probe are capable of hybridizing to each other, and the hybridization can result in quenching of fluorescence from the fluorophore by the fluorescence quencher. The detection probe/quencher oligonucleotide can have different structures under different conditions, and this can be reflected by the fluorescence change. When the detection probe and quencher oligonucleotide hybridize to each other in a stable double-stranded structure, the fluorophore and the quencher are in close proximity. The fluorophore can be quenched by the quencher and the detection probe becomes non-fluorescent at the emission wavelength of the fluorophore. When under denaturing conditions, such as under high temperature, the detection probe and quencher oligonucleotide are separated, and the fluorophore become fluorescent. In the presence of the target amplification product under suitable conditions, the detection probe can spontaneously bind to a strand of the amplification product, in lieu of the quencher oligonucleotide, and the fluorophore becomes fluorescent. In some forms, the detection probe forms a thermodynamically more stable duplex with the target amplification product as compared to the quencher oligonucleotide.

In some forms, the 3′ end of the quencher oligonucleotide and/or the detection probe is blocked from being extendable by a polymerase. Suitable blockers include a phosphate group, quencher moiety, or biotin. In some forms, the 3′ end of the quencher oligonucleotide is blocked from being extendable by a polymerase with a quencher moiety (e.g., Black Hole Quencher® (BHQ®)). In some forms, the 3′ end of the detection probe is phosphorylated.

Both the fluorophore and the fluorescence quencher can be on the terminal or internal bases of the detection probe or quencher oligonucleotide. In some forms, they are on opposed terminal complementary bases of the two strands. Thus, in some forms, the fluorophore can be attached to the 5′ or 3′ end of the detection probe, and/or the fluorescence quencher can be attached to the 5′ or 3′ end of the quencher oligonucleotide. In some forms, the fluorophore is attached to the 5′ end of the detection probe. In some forms, the fluorescence quencher is attached to the 3′ end of the quencher oligonucleotide.

In some forms, the detection probe and quencher oligonucleotide are unlinked, and thus two separate oligonucleotides

In some forms, a primer is also a probe. By fluorescently labeling one or more primers, the strategies disclosed herein of indirectly measuring amplification of the target by measuring unbound or unused probe can be carried out using primers only that also serve a probe, and without an additional probe. Thus, the compositions can also contain one or more fluorescently-labelled primers (e.g., forward and/or reverse primers). This/these primer(s) (e.g., forward or reverse primers) can be used in amplifying nucleic acid molecules in accordance with the disclosed methods. In some forms, the fluorescently labelled forward or reverse primers can be universal or common primers (e.g., the same forward and reverse primers can be used for the amplification of two or more distinct targets). In other forms, the forward and reverse primers are target-specific.

In some forms, fluorescently labelled forward or reverse primers contains the tag sequence at 5′ end and target specific binding sequence at the 3′ end.

Primers are typically at least 10, 15, 18, 20, 25, 30, 40, 50, or 60 nucleotides in length. In some forms, fluorescently-labeled primers are preferably between about 25 to about 45 nucleotides in length, and more preferably between about 30 to about 40 nucleotides in length (e.g., 31 or 39 nucleotides). However, there is no standard primer length for optimal hybridization or amplification. An optimal length for a particular primer application may be readily determined by those of skill in the art.

In some forms, the fluorescently-labelled forward primer or reverse primers are designed to have a Tm of about 66-74° C., inclusive.

In some forms, the fluorescently-labelled forward primer can be used to prime the qPCR (or dPCR or isothermal) reaction as well as used to measure amount of unused primer at each qPCR (or dPCR or isothermal) cycle.

The disclosed oligonucleotides can be modified at the base moiety, sugar moiety, or phosphate backbone, and may include other appending groups or labels, so long as the intended function is not compromised.

For example, the oligonucleotides may include one or more modified base moieties such as 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, and 2-methylguanine.

Suitable modified sugar moieties include arabinose, 2-fluoroarabinose, xylulose, and hexose. Suitable phosphate backbone modifications include phosphorothioate, phosphorodithioate, phosphoramidothioate, phosphoramidate, phosphordiamidate, methylphosphonate, alkyl phosphotriester, and a formacetal or analog thereof.

In some forms, the disclosed oligonucleotides and methods related thereto utilize the principle of molecular energy transfer (MET) and, preferably, fluorescence resonance energy transfer (FRET). In some forms, the disclosed oligonucleotides and methods related thereto utilize the principle of static or contact quenching, molecular energy transfer, and FRET. When an acceptor fluorophore is brought closer to a donor fluorophore (e.g., 20-100 Å), the intensity of fluorescence of the acceptor fluorophore increases, whereas the intensity of the fluorescence of donor fluorophore decreases due to an increased efficiency of fluorescence resonance energy transfer (FRET) from donor to acceptor fluorophore. When these two moieties are brought even closer, the intensity of both donor and acceptor fluorophores is reduced, which is called static or contact quenching. At these intimate distances, most of the absorbed energy is dissipated as heat and only a small amount of energy is emitted as light. For example, adjacent probes and TaqMan probes use the FRET mechanism, wherein the distance between donor and acceptor moieties causes FRET quenching. On the other hand, in competitive hybridization probes and molecular beacons, when the probe is not hybridized to the target, the two fluorescent moieties are very close to each other causing contact or static quenching (see, e.g., Marras S A, et al., Nucleic Acids Res., 30(21):e122 (2002)). One of the useful features of contact quenching is that all fluorophores are quenched equally well, irrespective of whether the emission spectrum of the fluorophore overlaps the absorption spectrum of the quencher, one of the key conditions that determines the efficiency of FRET.

In some forms, the methods rely on contact quenching since the detection probes and corresponding quencher oligonucleotides are designed to hybridize to each other, such that the fluorophore and fluorescence quencher are in close proximity.

The oligonucleotides can be labeled with a donor and/or an acceptor moiety. In some forms, the acceptor moiety may simply quench the emission of the donor moiety, or it may itself emit energy upon excitation by emission from the donor moiety. In a some forms, the donor moiety is a fluorophore, and the acceptor moiety may or may not be a fluorophore, such that fluorescent energy emitted by the donor moiety is absorbed by the acceptor moiety. In a some forms, the acceptor moiety is a fluorescence quencher.

A fluorescence quencher can quench a signal from the fluorophore to various degrees. For example, in some forms, the fluorescence signal detected in the presence of the fluorescence quencher can be 90% or less, 80% or less, 70% or less, 60% or less, 50% or less, 40% or less, 30% or less, 20% or less, 15% or less, 10% or less, or 5% or less of the signal detected in the absence of the fluorescence moiety. In some forms, no signal (e.g., above background) is detected in the presence of the fluorescence quencher.

In some forms, a suitable fluorophore is selected from: an Alexa Fluor® dye, an ATTO™ dye (e.g., ATTO™ 390, ATTO™ 425, ATTO™ 465, ATTO™ 488, ATTO™ 495, ATTO™ 514, ATTO™ 520, ATTO™ 532, ATTO™ Rho6G, ATTO™ 542, ATTO™ 550, ATTO™ 565, ATTO™ Rho3B, ATTO™ Rho11, ATTO™ Rho12, ATTO™ Thio12, ATTO™ Rho101, ATTO™ 590, ATTO 594, ATTO™ Rho13, ATTO™ 610, ATTO™ 620, ATTO™ Rho14, ATTO™ 633, ATTO™ 647, ATTO™ 647N, ATTO™ 655, ATTO™ Oxa12, ATTO™ 665, ATTO™ 680, ATTO™ 700, ATTO™ 725, ATTO™ 740), a DyLight® dye, a cyanine dye (e.g., Cy™ 2, Cy3™, Cy™ 3.5, Cy™ 3b, Cy5™, Cy™ 5.5, Cy™ 7, Cy™ 7.5), a FluoProbes dye, a Sulfo Cy™ dye, a Seta™ dye, an IRIS™ Dye, a SeTau™ dye, an Srfluor dye, a Square dye, fluorescein (FITC), tetramethylrhodamine (TRITC). Examples of Alexa Fluor® dyes include, but are not limited to: Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

In some forms, the fluorophore is selected from fluorescein (FAM™), hexachloro-fluorescein (HEX™), 2-chloro-7′-phenyl-1,4-dichloro-6-carboxy-fluorescein (VIC®), 5′-Dichloro-Dimethoxy-Fluorescein (JOE™), tetrachlorofluorescein (TET™), SUN™, tetramethylrhodamine (TAMRA™), QUASAR®670, CAL Fluor® Orange (CF560), CAL Fluor® Red 610 (CF610), and Texas Red® (Sulforhodamine 101 acid chloride).

Examples of fluorescence quenchers include, but are not limited to: a dark quencher, a Black Hole Quencher® (BHQ®) (e.g., BHQ®-0, BHQ®-1, BHQ®-2, BHQ®-3), a Qx1 quencher, an ATTO™ quencher (e.g., ATTO™ 540Q, ATTO™ 580Q, and ATTO™ 612Q), dimethylaminoazobenzenesulfonic acid (Dabsyl), Iowa Black® RQ, Iowa Black® FQ, IRDye® QC-1, a QSYR dye (e.g., QSYR 7, QSYR 9, QSY® 21), AbsoluteQuencher™, and Eclipse™. In some forms, a fluorescence quencher is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety.

Other suitable fluorescence quenchers are known in the art and include, without limitation, 1,4-bis-(3-hydroxy-propylamino)-anthraquinone, 1-(3-(4,4′-dimethoxy-trityloxy)propylamino)-4-(3-hydroxypropylamino)-anthraquinone, 1-(3-(2-cyanoethoxy(diisopropylamino)phosphinoxy)propylamino)-4-(3-(4,4′-dimethoxy-trityloxy)propylamino)-anthraquinone (#Q1), 1,5-bis-(3-hydroxy-propylamino)-anthraquinone, 1-(3-hydroxypropylamino)-5-(3-(4,4′-dimethoxy-trityloxy)propylamino)-anthraquinone, 1-(3-(cyanoethoxy(diisopropylamino)phosphinoxy)propylamino)-5-(3-(4,4′-dimethoxy-trityloxy)propylamino)-anthraquinone (#Q2), 1,4-bis-(4-(2-hydroxyethyl)phenylamino)-anthraquinone, 1-(4-(2-(4,4′-dimethoxy-trityloxy)ethyl)phenylamino)-4-(4-(2-hydroethyl)phenylamino)-anthraquinone, 1-(4-(2-(2-cyanoethoxy(diisopropylamino)phosphinoxy)ethyl)phenylamino)-4-(4-(2-(4,4′-dimethoxy-trityloxy)ethyl)phenylamino)-anthraquinone, 1,8-bis-(3-hydroxy-propylamino)-anthraquinon, and 4-((4-(dimethylamino)phenyl)azo)benzoic Acid (Dabcyl).

In some forms, it can be advantageous to use different quenchers attached to the same quencher oligonucleotide (e.g., for the detection of two or more targets in multicolor multiplexing). For example, BHQ®-1, BHQ®-2, and BHQ®-3 can quench different fluorophores. For example, FAM™ can be quenched by BHQ®-1 whereas Quasar670 can be quenched by either BHQ®-2 or BHQ®-3. In some forms, the fluorophore quencher is selected from BHQ®-1, BHQ®-2, and BHQ®-3.

In some forms, PCR using SYBR Green as a detection method for amplifying and quantifying DNA can also be performed. SYBR Green is a fluorescent dye that binds non-specifically to double-stranded DNA during amplification. As the DNA is amplified during the PCR cycles, the dye intercalates into the newly formed double-stranded DNA, resulting in an increase in fluorescence intensity that correlates with the amount of amplified DNA.

ii. Next Generation Sequencing (NGS)

In some forms, the oligonucleotide barcodes are prepared for sequencing, such as next-generation sequencing (NGS). This involves labeling the oligonucleotide barcodes with unique identifiers corresponding to different samples or targets, which are then pooled and sequenced. The NGS platform identifies and quantifies the barcodes, providing a sensitive, multiplexed readout of the target antigen levels in the sample. In these forms, each analyte can be identified using a unique DNA barcode, such that hundreds to thousands of analytes can be detected/profiled using a single kit/assay. These methods ensure accurate detection and quantification of target antigens with high sensitivity and specificity.

In some forms, the NGS platform includes, but is not limited to, Illumina Sequencing, Ion Torrent Sequencing, PacBio Sequencing (SMRT), Oxford Nanopore Sequencing, Roche 454 Sequencing, SOLID Sequencing, BGI/MGI Sequencing (DNBSeq), and Helicos Sequencing.

TABLE 3
Exemplary Reagents for NGS test (showing target, catalog number and vendor)

| Target | DAB (DNA barcoded) | Vendor | CAB (Biotinylated) | Vendor | Antigen | Vendor |
|---|---|---|---|---|---|---|
| IL-8 | 130-10975 | Raybio | 130-10985B | Raybio | 230-00010 | Raybio |
| ALPP | 130-10518 | Raybio | 130-10519B | Raybio | 230-00257 | Raybio |
| CD38 | 130-10323 | Raybio | 130-10324B | Raybio | 230-00549 | Raybio |
| SOD1 | 130-10369 | Raybio | 130-10366B | Raybio | 230-00587 | Raybio |
| VCAN | 130-10778 | Raybio | 130-10773B | Raybio | 230-30109 | Raybio |

The nucleic acid substrate for sequencing can be the nucleic acids from the original sample and/or an amplified nucleic acid product thereof, e.g., produced by a qPCR protocol. In various aspects, the analysis can include sequencing the nucleic acids using, but not limited to, Sanger sequencing, single molecule real-time (SMRT) sequencing, nanopore DNA sequencing, massively parallel signature sequencing (MPSS), colony sequencing, 454 pyrosequencing, Illumina sequencing, combinatorial probe anchor synthesis (cPAS), SOLID sequencing, ion torrent semiconductor sequencing, DNA nanoball sequencing, heliscope single molecule sequencing, or a combination of any thereof, optionally using a microfluidic system. In some embodiments, the sequencing is or includes Illumina, Ion Torrent, PacBio, or Oxford Nanopore technology, or any similar next-generation sequencing platforms.

For comprehensive analysis, raw nucleic acids and/or PCR products can be analyzed using sequencing, such as next-generation sequencing (NGS). This approach allows detailed profiling of gene expression and identification of analytes. The versatility of this workflow accommodates various sequencing platforms, including but not limited to Illumina, Ion Torrent, PacBio, and Oxford Nanopore Technologies, nanostring nCounter, and Miser sequencing (i.e., sequencing PCR from illumination).

In some embodiments, the molecular analysis of analytes involves sample indexing, adapter ligation, and/or library normalization. Sample indexing (“barcoding”) is used to assign unique identifiers to multiple targets and/or samples, such as hundreds to thousands of targets and/or samples, allowing them to be processed together on high-throughput sequencing platforms. Adapter ligation can be performed according to sequencing platform-specific protocols, adding platform-compatible end sequences and unique index sequences to DNA or RNA fragments specific to kidney biomarkers. Libraries can be prepared at concentrations suitable for sequencing, with amplification or dilution performed to achieve the required DNA or RNA concentration. The concentration of nucleic acids in the library can be measured using methods such as quantitative real-time PCR (qPCR) or fluorescence-based instruments like the ThermoFisher Qubit. The sequencing library can include any of amplified DNA, RNA, or cDNA fragments. Sequencing platforms, including Illumina, Oxford Nanopore, and PacBio, can then be used to generate reads that provide detailed information about gene expression, transcript abundance, and molecular patterns related to kidney injury or disease progression. This workflow allows for multiplexed analysis of analytes from multiple samples in a single sequencing run, supporting comprehensive profiling and detection of analytes.

In some forms, the method of sequencing such as next-generation sequencing (NGS) disclosed herein can be used for simultaneous detection and analysis of five or more analytes across single and/or multiple samples, optionally hundreds to thousands of analytes, such as from 100 to 10,000 analytes, across single and/or multiple samples.

In some forms, the NGS methodology used in the disclosed method can facilitate simultaneous detection and analysis of multiple analytes within a single sample, providing comprehensive characterization of numerous analytes from a single sample. For example, the NGS approach in the disclosed method can be used for the analysis of 2, 5, 10, 50, 100, 500, 1,000, 5,000, 10,000 or more target analytes, such as from 10 to 10,000, from 100 to 10,000, from 10 to 1000, or from 100 to 1000 target analytes, in a single sample, in a single sequencing run.

In some forms, the NGS approach facilitates simultaneous processing across multiple separate samples, where each sample contains one or more target analytes. For example, the NGS approach in the disclosed method can be used for the analysis of 2, 5, 10, 50, 100, 500, 1,000, 5,000, or more separate samples in a single sequencing run, where each sample potentially containing multiple target analytes, such as from 10 to 10,000, from 100 to 10,000, from 10 to 1000, or from 100 to 1000 target analytes.

### 6. Determining Concentration

Optionally, the method includes a step of determining the concentration of target analytes. The concentration of the target antigen is determined by amplifying the eluted DNA barcode using qPCR. A PCR master mix, oligonucleotide primers specific to the barcode, and either SYBR Green or TaqMan probe-based detection are used to quantify the DNA. Fluorescence data are recorded during amplification, and the resulting Ct (cycle threshold) values are compared to a standard curve generated using known concentrations of the target antigen. Lower Ct values correspond to higher antigen concentrations in the original sample. This quantitative analysis allows accurate and sensitive determination of protein levels.

Standard curves are generated by running a set of known concentrations of the target antigen or antigens alongside the samples during the BIQ-ELISA process. These known concentrations are prepared by serial dilution of a standard stock solution, typically spanning a wide range of values to cover the expected concentrations in the samples. Each standard is processed in the same way as the test samples, including incubation, washing, elution, and qPCR amplification.

During qPCR, fluorescence data are collected in real-time, and the cycle threshold (Ct) values are determined for each standard. The Ct value, which represents the number of PCR cycles required for the fluorescence signal to cross a predefined threshold, is inversely proportional to the concentration of the antigen. Lower Ct values indicate higher antigen concentrations.

To generate the standard curve, the Ct values (y-axis) are plotted against the logarithm of the known concentrations of the standard solutions (x-axis). A linear regression is performed to fit the data points, producing an equation of the form: y=mx+b, where y is the Ct value, x is the log concentration, m is the slope of the line, and b is the y-intercept.

For determining the concentration of unknown samples, their Ct values are measured and input into the standard curve equation. The resulting x-value is the log concentration of the target antigen in the sample. This value is then exponentiated (antilog) to calculate the actual concentration. The standard curve ensures accurate quantification of target antigens by providing a reference for converting Ct values into precise concentrations.

The disclosed compositions and methods can be further understood through the following numbered paragraphs.

1. A kit for detection of a target analyte in a sample, the kit comprising:
(a) a first reagent; and
(b) a second reagent,
wherein:
(i) the first reagent comprises magnetic particles modified with a first binding agent specific for the analyte and
(ii) the second reagent comprises a second binding agent specific for the analyte, conjugated to a nucleic acid barcode.

2. The kit of paragraph 1, wherein the first binding agent and the second binding agent bind to different epitopes on the target analyte.

3. A kit for detection of two or more target analytes in a sample, the kit comprising:
(a) modified magnetic particles comprising two or more first binding agents; and
(b) two or more second binding agent,
wherein the two or more first binding agents and the two or more second binding agents form two or more binding sets,
wherein each binding set comprises one first binding agent and one second binding agent,
wherein the first binding agent and the second binding agent in each binding set are specific for the same target analyte,
wherein each second binding agent is conjugated to a nucleic acid barcode, and
wherein the two or more analytes are different from each other.

4. The kit of any one of paragraphs 1-3, wherein the first binding agent and the second binding agent in each binding set bind to different epitopes or the same epitope on the target analyte.

5. The kit of any one of paragraphs 1-4, wherein the first binding agent and the second binding agent are independently an antibody, an aptamer, or a peptide, or a combination thereof.

6. The kit of any one of paragraphs 1-5, wherein the modified magnetic particle has a diameter of about 0.5 μm to about 5 μm, 0.3 μm-5.5 μm, 0.2 μm-6 μm, 1 μm-4.5 μm, 1.5 μm-4 μm, 2 μm-3.5, μm, or 2.5 μm-3 μm.

7. The kit of any one of paragraphs 1-6, wherein the modified magnetic particle is provided in the form of an aqueous suspension at a density of about 1.4 g DS/cm³-1.8 g DS/cm³, 1.3 g DS/cm³-1.9 g DS/cm³, 1.5 g DS/cm³-1.7 g DS/cm³, 1.2 g DS/cm³-2.0 g DS/cm³, 1.6 g DS/cm³-1.9 g DS/cm³, 1.1 g DS/cm³-2.1 g DS/cm³, or 1.0 g DS/cm³-2.5 g DS/cm³.

8. The kit of any one of paragraphs 1-7, wherein the first binding agent is conjugated to the modified magnetic particle via an affinity binding pair selected form a group consisting of avidin-family protein/biotin, aptamer/target molecule pairs, receptor/ligand pairs, natural or synthetic receptor/ligand pairs, or amines and carbonyl compounds.

9. The kit of paragraph 8, wherein the avidin-family protein is streptavidin, avidin, or neutravidin.

10. The kit of paragraph 5, wherein the antibody is a monoclonal antibody, a polyclonal antibody, a recombinant antibody, a humanized antibody, a human antibody, a chimeric antibody, a functional fragment thereof, or a combination thereof.

11. The kit of any one of paragraphs 1-10, wherein the analyte is selected from the group consisting of IL-8 (Interleukin-8), ALPP (Alkaline phosphatase for example, placental type (EC 3.1.3.1)), CD38 (Cluster of Differentiation 38), SOD1 (Superoxide dismutase-1), and VCAN (Versican).

12. The kit of paragraph 11, comprising DAB/CAB pairs selected from Table 2, optionally herein the nucleic acid barcode is selected from the group consisting of SEQ ID NO: 1-5 and SEQ ID NO: 20-24.

13. The kit of any one of paragraphs 1-12, wherein the analyte is in a sample, optionally wherein the sample is a food sample, an environmental sample, an industrial sample, a human clinical sample, a veterinary sample, and other samples of biological origin, optionally wherein the sample is blood, serum, plasma, feces, mucosal swab, tissue aspirate, tissue homogenate, cell culture, cell culture supernatant (including cultures of eukaryotic and prokaryotic cells), urine, saliva, sputum, and/or cerebrospinal liquid.

14. The kit of paragraph 10, wherein the functional fragment is a single-chain variable fragment (scFv), a Fab fragment, a nanobody, a bispecific antibody, a diabody, a triabody, or a combination thereof.

15. The kit of any one of paragraphs 1-14, wherein the second binding agent is conjugated to a nucleic acid barcode via a linker.

16. The kit of paragraph 15, wherein the linker is

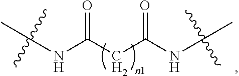

wherein n1 is an integer from 1 to 10, from 1 to 8, from 1 to 6, from 2 to 10, from 2 to 8, from 4 to 10, or from 4 to 8, such as 6; and wherein the secondary amino groups are from the second binding agent and the nucleic acid barcode respectively.

17. The kit of any one of paragraphs 1-16, wherein the nucleic acid barcode comprises:
(a) a region for hybridization with a TaqMan probe;
(b) a region for primer binding during PCR amplification; and
(c) a region for target specificity.

18. The kit of any one of paragraphs 1-17, further comprising one or more buffers and one or more reagents.

19. The kit of paragraph 18, wherein one or more buffers comprise:
(a) a dilution buffer for preparing standards and diluting samples;
(b) a wash buffer for removing unbound materials from the magnetic particles;
(c) an elution buffer comprising sodium hydroxide for releasing the ONA from the immune complex;
(d) a blocking buffer for reducing non-specific binding

20. The kit of paragraph 18 wherein one or more reagents comprise:
(a) a PCR master mix comprising DNA polymerase and nucleotides for qPCR amplification;
(b) one or more oligonucleotide primers for amplifying the ONA barcode;
(c) a TaqMan probe labeled with a fluorescent reporter and quencher for detecting the ONA during qPCR amplification.

21. The kit of any one of paragraphs 1-20, further comprising instructions for use.

22. A method of detecting one or more analytes in a sample using the kit of paragraphs 1-21, the method comprising:
(i) incubating the sample with the modified magnetic particles and the second binding agent to form an immune complex; and
(ii) performing signal amplification and/or sequencing of the nucleic acid barcode.

23. The method of paragraph 22, further comprising, after step (i), (a) purifying the immune complex.

24. The method of paragraph 23, wherein step (a) comprises washing the immune complex with a wash buffer to remove unbound materials and separating the immune complex from the buffer using a magnetic field or centrifugation.

25. The method of any one of paragraphs 22-24 further comprising, after step (i), eluting the nucleic acid barcode from the immune complex using an elution buffer.

26. The method of any one of paragraphs 22-25, wherein step (ii) comprises adding a PCR mix to the nucleic acid barcode and amplifying the eluted nucleic acid using qPCR.

27. The method of any one of paragraphs 22-26, further comprising, after step (ii), (b) determining a concentration of each of the target analyte.

28. The method of paragraphs 27, wherein step (b) comprises generating a standard curve for each analyte and calculating the analyte concentration in the sample based on Ct values obtained from step (ii).

29. The method of any one of paragraphs 22-28, wherein step (ii) is performed using qPCR or sequencing, or a combination thereof.

30. The method of any one of paragraphs 22-29, wherein the sequencing is performed using next-generation sequencing (NGS), such as Illumina sequencing, Ion Torrent semiconductor sequencing, PacBio SMRT sequencing, Oxford Nanopore sequencing, nanostring nCounter, and/or Miser sequencing.

31. The method of paragraph 30, wherein the NGS comprises simultaneous analysis and detection of five or more analytes across single and/or multiple samples, optionally hundreds to thousands of analytes, such as from 100 to 10,000 analytes, across single and/or multiple samples.

The disclosed compositions and methods can be further understood through the following numbered paragraphs.

32. A kit for detection of a target analyte in a sample, the kit comprising:
(a) a first reagent; and
(b) a second reagent,
wherein:
(i) the first reagent comprises magnetic particles modified with a member of an affinity binding pair, and
(ii) the second reagent comprises a second binding agent specific for the analyte and a nucleic acid barcode, wherein the second binding agent is conjugated to the nucleic acid barcode.

33. A kit for detection of two or more target analytes in a sample, the kit comprising:
(a) a first reagent; and
(b) a second reagent,
wherein:
(i) the first reagent comprises magnetic particles modified with a member of an affinity binding pair; and
(b) the second reagent comprises two or more second binding agents, each specific to a target analyte, wherein each second binding agent is conjugated to a nucleic acid barcode,
wherein the two or more target analytes are different from each other, and
wherein the nucleic acid barcode for each target analyte is unique to the target analyte.

34. The kit of paragraph 32 or 33, wherein the modified magnetic particle has a diameter of about 0.5 μm to about 5 μm, 0.3 μm-5.5 μm, 0.2 μm-6 μm, 1 μm-4.5 μm, 1.5 μm-4 μm, 2 μm-3.5, μm, or 2.5 μm-3 μm.

35. The kit of any one of paragraphs 32-34, wherein the modified magnetic particle is provided in the form of an aqueous suspension at a density of about 1.4 g DS/cm³-1.8 g DS/cm³, 1.3 g DS/cm³-1.9 g DS/cm³, 1.5 g DS/cm³-1.7 g DS/cm³, 1.2 g DS/cm³-2.0 g DS/cm³, 1.6 g DS/cm³-1.9 g DS/cm³, 1.1 g DS/cm³-2.1 g DS/cm³, or 1.0 g DS/cm³-2.5 g DS/cm³.

36. The kit of any one of paragraphs 32-35, wherein the analyte is in a sample, optionally wherein the sample is a food sample, an environmental sample, an industrial sample, a human clinical sample, a veterinary sample, and other samples of biological origin, optionally wherein the sample is blood, serum, plasma, feces, mucosal swab, tissue aspirate, tissue homogenate, cell culture, cell culture supernatant (including cultures of eukaryotic and prokaryotic cells), urine, saliva, sputum, and/or cerebrospinal liquid.

37. The kit of any one of paragraphs 32-36, wherein the nucleic acid barcode comprises:
(a) a region for hybridization with a TaqMan probe;
(b) a region for primer binding during PCR amplification; and
(c) a region for target specificity.

38. The kit of any one of paragraphs 32-37, further comprising one or more buffers and one or more reagents.

39. The kit of paragraph 38, wherein the one or more buffers comprise:
(a) a dilution buffer for preparing standards and diluting samples;
(b) a wash buffer for removing unbound materials from the magnetic particles;
(c) an elution buffer comprising sodium hydroxide for releasing the ONA from the immune complex; and/or
(d) a blocking buffer for reducing non-specific binding.

40. The kit of paragraph 38 or 39, wherein the one or more reagents comprise:
(a) a PCR master mix comprising DNA polymerase and nucleotides for qPCR amplification;
(b) one or more oligonucleotide primers for amplifying the ONA barcode; and/or
(c) a TaqMan probe labeled with a fluorescent reporter and quencher for detecting the ONA during qPCR amplification.

41. The kit of any one of paragraphs 32-40, further comprising instructions for use.

42. The kit of any one of paragraphs 32-41, further comprising a protein labeling reagent.

43. The kit of any one of paragraphs 32-42, wherein the member of the affinity binding pair is an avidin-family protein.

44. The kit of paragraph 43, wherein the avidin family protein is streptavidin, avidin, or neutravidin.

45. The kit of any one of paragraphs 42-44, wherein the protein labeling reagent is a biotinylation reagent.

46. The kit of any one of paragraphs 32-45, wherein the nucleic acid barcode is selected from the group consisting of SEQ ID NO:1-5, SEQ ID NO:34, and SEQ ID NO:35.

47. The kit of any one of paragraphs 32-46, wherein the analyte is selected from the group consisting of MIP-1a, Eotaxin, IL-22, IL-12p40, IL-12p70, EXN4, IFN-γ, IL-1β, IL-6, and TNF-α.

48. The kit of any one of paragraphs 32-47, wherein the second binding agent is conjugated to the nucleic acid barcode via a linker.

49. The kit of paragraph 48, wherein the linker is

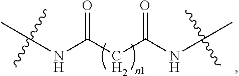

wherein n1 is an integer from 1 to 10, from 1 to 8, from 1 to 6, from 2 to 10, from 2 to 8, from 4 to 10, or from 4 to 8, such as 6; and wherein the secondary amino groups are from the second binding agent and the nucleic acid barcode respectively.

50. The kit of any one of paragraphs 32-49, wherein the second binding agent is an antibody, an aptamer, or a peptide, or a combination thereof.

51. The kit of paragraph 50, wherein the antibody is a monoclonal antibody, a polyclonal antibody, a recombinant antibody, a humanized antibody, a human antibody, a chimeric antibody, a functional fragment of antibody, or a combination thereof.

52. The kit of paragraph 51, wherein the functional fragment of antibody is a single-chain variable fragment (scFv), a Fab fragment, a nanobody, a bispecific antibody, a diabody, a triabody, or a combination thereof.

53. A method of detecting one or more analytes in a sample using the kit of any one of paragraphs 42-52, the method comprising:
(i) labeling the one or more analytes with the protein labeling reagent;
(ii) incubating the sample with the modified magnetic particles and the second reagent to form an immune complex; and
(iii) performing signal amplification and/or sequencing of the nucleic acid barcode.

54. The method of paragraph 53, wherein the protein labeling reagent is a biotinylation reagent.

55. The method of paragraph 53 or 54, further comprising, after step (ii), (a) purifying the immune complex.

56. The method of any one of paragraphs 53-55, wherein step (a) comprises washing the immune complex with a wash buffer to remove unbound materials and separating the immune complex from the buffer using a magnetic field or centrifugation.

57. The method of any one of paragraphs 53-56, further comprising, after step (ii), eluting the nucleic acid barcode from the immune complex using an elution buffer.

58. The method of any one of paragraphs 53-57, wherein step (iii) comprises adding a PCR mix to the nucleic acid barcode and amplifying the eluted nucleic acid using qPCR.

59. The method of any one of paragraphs 53-58, further comprising, after step (iii), (b) determining a concentration of each of the target analyte.

60. The method of paragraph 59, wherein step (b) comprises generating a standard curve for each analyte and calculating the analyte concentration in the sample based on Ct values obtained from step (iii).

61. The method of any one of paragraphs 53-60, wherein step (iii) is performed using qPCR or sequencing, or a combination thereof.

62. The method of any one of paragraphs 53-61, wherein the sequencing is performed using next-generation sequencing (NGS), such as Illumina sequencing, Ion Torrent semiconductor sequencing, PacBio SMRT sequencing, Oxford Nanopore sequencing, nanostring nCounter, and/or Miser sequencing.

63. The method of paragraph 62, wherein the NGS comprises simultaneous analysis and detection of five or more analytes across single and/or multiple samples, optionally hundreds to thousands of analytes, such as from 100 to 10,000 analytes, across single and/or multiple samples.

The disclosed compositions and methods can be further understood through the following non-limiting examples.

### EXAMPLES

### Example 1. Sequence Screening and Preparation Method of Oligonucleotide Acid (ONA) Barcode, as Well as its Conjugating Method with Antibodies

### Methods

### I. Preparation of Oligonucleotide (ONA) Barcode

The 5′ terminal amino modified oligonucleotide sequence ONA was purchased from IDT and consists of three parts: a PCR primer region shared at both ends (P1: 26 bp; P2: 34 bp) and a middle specific coding region (20 bp). After PCR amplification, the total length of the product was 80 bp

(SEQ ID NO: 1)
5′-/5AmMC6/AGCACACATTGCTGGTTCTGCTCACGT
CTGACTACAGGCATTCCGTCTGTCTCTTATACACATCT
CCGAGCCCACGAGAC-3′.

### II. Preparation of Antibody Oligonucleotide Conjugates (DAB-ONA)

1. Prepare HPLC pure oligonucleotides with amino modification at the 5′ end into a 200 μM solution using coupling buffer (200 mM HEPES, pH 8.5).

2. Prepare a 50 mM solution of bis(sulfosuccinimide) suberate (BS3) in dimethyl sulfoxide (DMSO).

3. Mix a 200 μM oligonucleotide solution and a 50 mM bis(sulfonylsuccinimide) octanoic acid ester solution in a volume ratio of 2:1 and shake. Allow to stand at room temperature for 30 minutes for activation.

During the waiting process, the pre-treatment desalination centrifuge column: centrifuge at 1000×g for 1 minute to remove the storage solution. Add coupling buffer to the column and centrifuge at 1000×g for 1 minute to remove the coupling buffer. Repeat the addition of coupling buffer and centrifuge once, then place the column in a new collection tube for later use.

After 5.30 minutes, the activated oligonucleotide mixture was added to a Zeba desalination column equilibrated with coupling buffer and centrifuged at 1000×g for 2 minutes to collect purified activated oligonucleotides.

6. Mix the purified activated oligonucleotides with the purified antibody to be conjugated in a ratio of 7:1 or 10:1 and shake. Let it stand overnight at room temperature until the coupling reaction is complete.

### III. Purification of Antibody Oligonucleotide Conjugates

1. Prepare FPLC buffer A (150 mM NaCl, 50 mM Tris HCl, pH 8.0) and buffer B (1 M NaCl, 50 mM Tris HCl, pH 8.0), filter through a 0.22 μm filter.

2. Dilute the mixture of antibody oligonucleotide coupling reaction moderately with buffer A.

3. Use buffer A to equilibrate the Nuvia HP Q chromatographic column. After the chromatographic column is equilibrated, load the diluted mixture of antibody oligonucleotide coupling reaction into the chromatographic column at a flow rate of 1 mL/min.

4. After the sample loading is completed, rinse an additional 2 column volumes with buffer A to allow the UV260 and 280 signals to return to baseline.

5. Elute at a flow rate of 1 mL/min in a gradient ratio of 0-100% buffer B within 16 column volumes, and collect chromatographic peaks.

6. Use a protein concentration tube to concentrate the collected peaks containing antibody oligonucleotide conjugates, and replace them with phosphate buffered saline (PBS) buffer to remove excess salt.

7. Add bovine serum albumin (BSA) and sodium azide (NaN3) as protective agents and store at 4° C. for a long time.

### IV. Preparation of Antibody Coated Magnetic Beads

1. Shaking Dynabeads™ The magnetic beads coated with M-280 streptavidin were thoroughly mixed for 30 seconds, and an appropriate volume of magnetic beads was transferred to a clean centrifuge tube and placed on a magnetic rack for 2 minutes. After the magnetic beads stabilize, carefully remove the supernatant.

2. Use phosphate buffered saline (PBST) containing 0.05% (v/v) Tween-20 to resuspend the magnetic beads to their original volume, shake them vigorously, and then place them back on the magnetic rack for 2 minutes. After the magnetic beads stabilize, carefully remove the supernatant. Repeat washing once to remove the supernatant.

3. Add an appropriate amount of biotinylated antibody to be coated according to the ratio of 15 μg biotinylated antibody per 100 μl magnetic beads, shake and mix well

4. Incubate magnetic beads and antibodies at a speed of 1000 revolutions per minute for 30 minutes at room temperature

5. After incubation, place the centrifuge tube back on the magnetic rack for 2 minutes. After the magnetic beads stabilize, carefully remove the supernatant.

6. Wash the magnetic beads twice with PBST to remove uncoated antibodies

7. Resuspend magnetic beads in phosphate buffer containing bovine serum albumin (BSA) and sodium azide (NaN3) in the same volume as the original, and store them at 4° C. for a long time.

### V. Testing Crosslinkers

### CrossLinker 1: Sulfo-SMCC

Properties: unique direction and commonly used in conjugation

Protocol:
1. Use 100-fold molar excess TCEP to reduce the thiol-modified DNA oligo, incubate 30 min at RT
2. Purify the reduced DNA oligo containing thiol group (—SH) by ethanol precipitation
3. Activate the antibody by using 10-fold molar excess Sulfo-SMC, incubate 30 min at RT
4. Purify the activated antibody by desalting column
5. Combine the activated antibody and reduced oligo together and incubate overnight

Estimate the conjugation efficiency by using streptavidin to replace the antibody. Conjugate streptavidin with 3-fold molar excess oligo. The percentage is the yield compared with the original input. The result indicated that most streptavidin was not conjugated with oligo, indicating the low efficiency. To test the oligo streptavidin ratio, a gradient of oligo was used to conjugated the same amount of streptavidin. The conjugates were used for SDS-PAGE. The gel was stained with SYBR Gold to visualized DNA. Higher oligo-protein ratio improves the yield of conjugates. To figure out the details about the conjugation reaction progress, samples were collected for analysis every 30 minutes. Longer incubation time improves the yield of conjugates. To improve the activation of streptavidin by Sulfo-SMCC, different incubation times, temperatures and solvent for Sulfo-SMCC were tested. But these conditions did not make obvious difference. As the conjugation efficiency by Sulfo-SMCC cannot be improved a lot, other methods were tested.

### CrossLinker 2: DBCO-Azide Reaction

Properties: unique direction and commonly used in conjugation

Drawback: reagent and the azide modified oligo are much more expensive than other methods.

Protocol:
1. Use 20-fold molar excess DBCO-ester to activate streptavidin, incubate at RT for 30 min
2. Remove excess DNCP-ester by desalting column
3. mix 3-fold molar excess azide modified oligo with the activated streptavidin incubated at 4° C. overnight

Two batches of streptavidin conjugated by DBCO method was made and analyzed by SDS-PAGE. However, although there are conjugates in batch 2, there is no conjugates in batch 1. In addition, the yield of conjugates in batch 2 is still lower than 50%. As the reagent cost is too high, this method may not be suitable to construct hundreds of barcoded DNA conjugated antibody.

### CrossLinker 3: DSS

Disuccinimidyl suberate (DSS) is a homobifunctional linker and both ends react with amine (—NH2) groups. This chemical does not dissolve in water. However, amine modified oligo and DSS are much cheaper.

Based on Li et al. 2019 (https://doi.org/10.1002/cbic.201900027), DSS could be a relevant crosslinker for conjugating antibody and oligos:
As amino-DNA is comparatively cheap, usually activate DNA first (in DMSO with the help of acetonitrile-triethylamine).
To avoid DNA dimers, overdose of DSS is used (250× or 500× molar excess).
Activated DNA can be purified by ethanol precipitation of HPLC.
About 3-4× molar excess of activated DNA is used for conjugating protein.
The conjugation happens at room temperature overnight.

As HPLC was not available, ethanol purification was first used to purify activated DNA. As both ends of DSS can react with oligo, it needs to identify the ratio of DSS:oligo to avoid produce too much oligo self-dimers. When the ratio of DSS:oligo is too low or too high, there is less oligo self-dimers. Here when DSS:oligo is 250, there is almost no oligo dimer produced. This ratio was used for following tests. As DSS can also make proteins to form self-complex, streptavidin was treated with different amount of DSS and the supernatant of activated oligo purification. To purify the activated oligo, it was precipitated using absolute ethanol and washed by 75% ethanol twice.

More DSS can activate more streptavidin to make lager bands. Even the washed supernatant, there is still too much DSS residue. To purify the activated oligo, it was precipitated using absolute ethanol and washed by 75% ethanol twice.

DSS can be relatively quantified based on the A260 change after hydrolysis. A standard curve was prepared. A DSS sample was prepared the same to the DNA activation reaction, where the final DSS concentration is 16.7 mM. After purified by desalting column once: 0.528 mM. After purified by desalting column twice: 0.161 mM. The expected concentration of DNA after purification is 0.2 mM with no lost. DSS residue is still too much. It is likely caused by the insolubility so that DSS cannot be trapped by desalting column. To improve the purification, BS3 was then used.

### CrossLinker 4: BS3

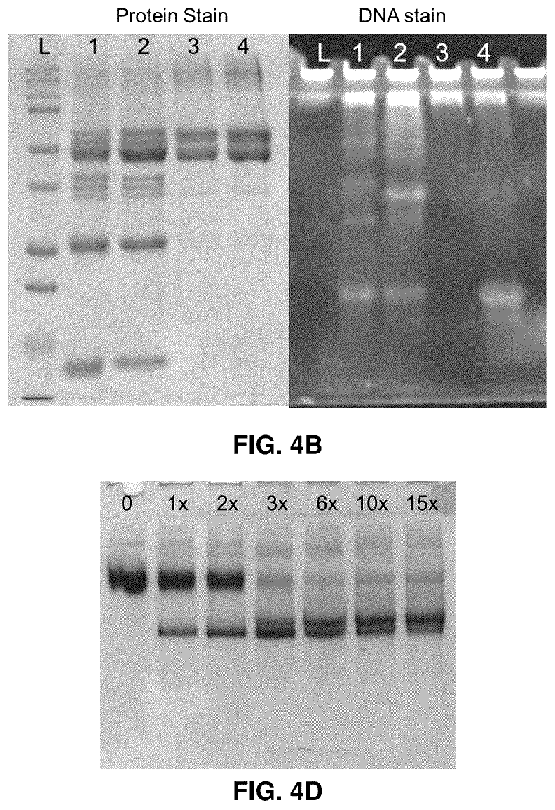

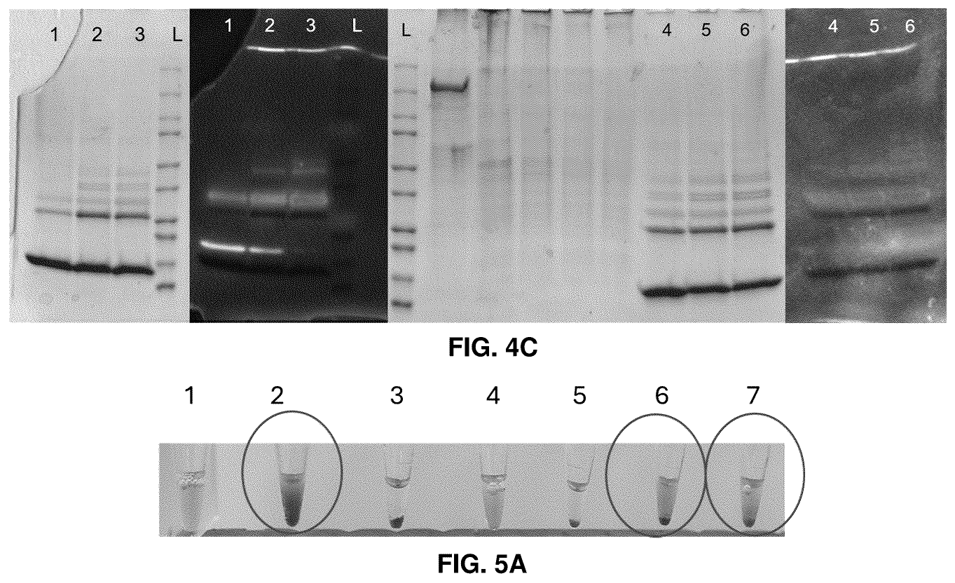

BS3 is the soluble version of DSS. Thus, the same parameter for DSS was used for BS3. BS3 has been identified to be a suitable crosslinker for linking binding agent to nucleotide barcodes (FIG. 4A-4D).

### VI. Test Streptavidin Beads

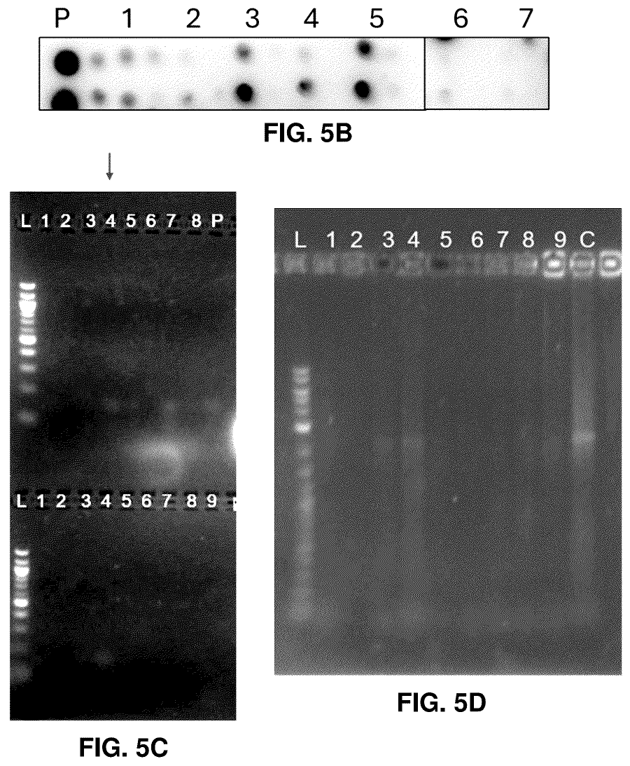

Beads tested as demonstrated in FIGS. 5A-5D:
1. HV0150 Hi-SurMag Streptavidin, 150 nm, 2 ml, 1 mg/ml (Custom)
2. MV1000-002 MonoMag Streptavidin, 1 um 2 ml (Custom)
3. HV1000-02 HiSur Streptavidin, 1 um, 2 ml (Custom)
4. 2574067 1 mL Dynabeads™ M-280 Streptavidin, 2.8 μm
5. 01342559 1 mL Dynabeads™ M-270 Streptavidin, 2.8 μm
6. 01342859 1 mL Dynabeads™ MyOne™ Streptavidin C1, 1 μm
7. 01340664 1 mL Dynabeads™ MyOne™ Streptavidin T1, 1 μm
8. Bioeast M2800S3-XC 2.8 um
9. Beaver 22307-1 1 um

To test the effect in PCR reaction:
5 ul bead each; wash 2× w/ PBS
Run PCR w/and w/o beads

Dynabeads M-280 is identified as the most suitable for downstream PCR.

TABLE 4
represents specifications for magnetic beads.

| Manufacturer | Category | Item number | concentration | particle size |
|---|---|---|---|---|
| BOYUE | COOH | M1000C3 | 50 mg/mL | 1.2 um |
| BOYUE | COOH | M2800C3 | 50 mg/mL | 2.8 um |
| BOYUE | SA | M2800S3-XC | 10 mg/ml | 2.8 um |
| BEAVER | SA | 22307-1 | 10 mg/ml | 1.0 um |
| BEAVER | COOH | 70109-1 | 10 mg/ml | 1.0 um |
| REBESI | COOH | NBL303-5 | 10 mg/m | 1.0 un |
| ISR | COOH | MS160 | 10 mg/mL | 1.6 um |
| SUZHOUWEIDU | COOH | CMP1001CA-1 | 25 mg/ml | 1.0 um |

VII. Comparing Magnetic Washer with Filer Plates

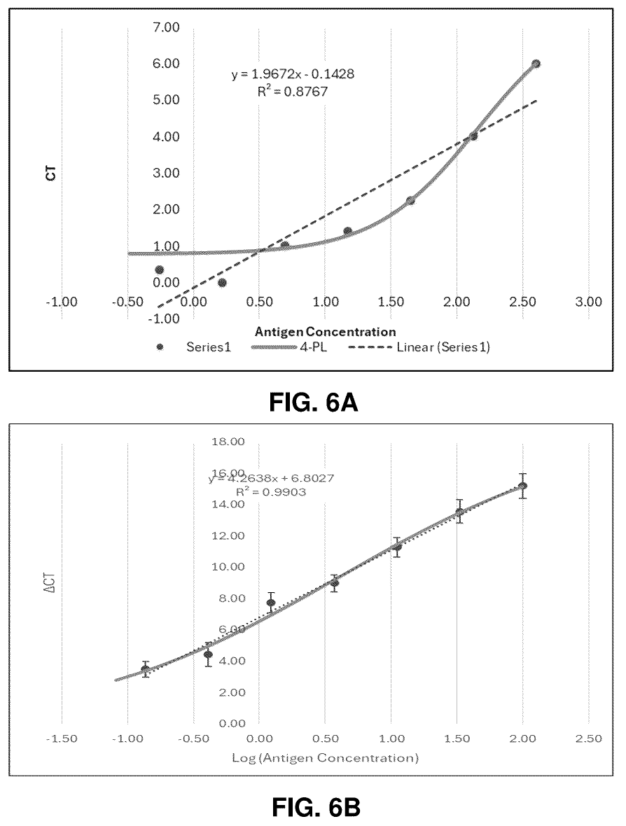

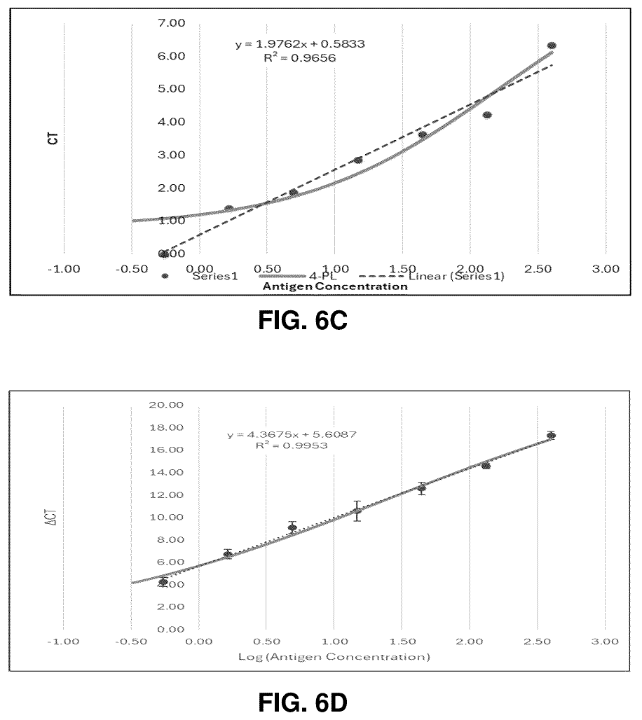

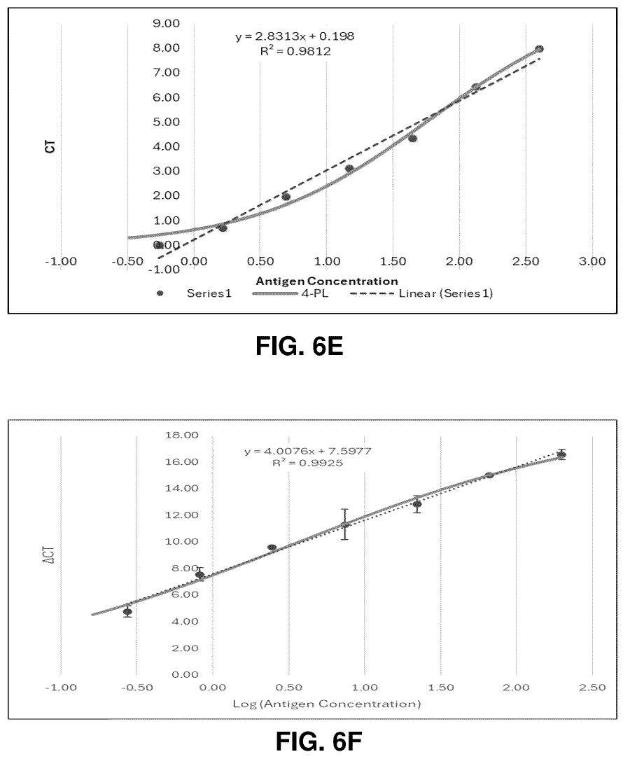

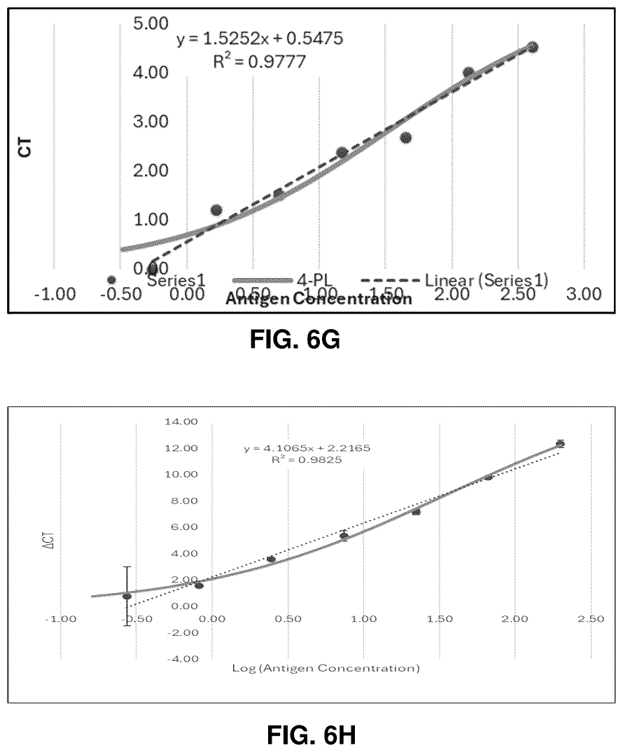

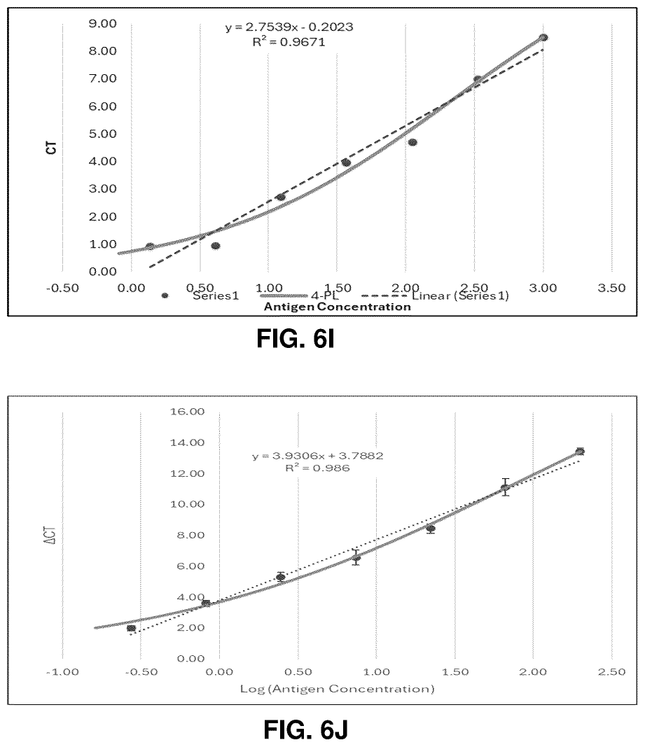

Before using Magnetic Bead Washer, Filter Plate was tested in Washing Steps. Seven different filter plates were tested. HV filter plate was proved to be the best. Assays on the filter plates were further characterized. HV Plate is the most suitable for lower background but higher delta CT. Direct mixing of reagents in prewet HV filter plate works the best. It was found that magnetic bead washer can increase the detection sensitivity through lowering the background (FIGS. 6A-6J).

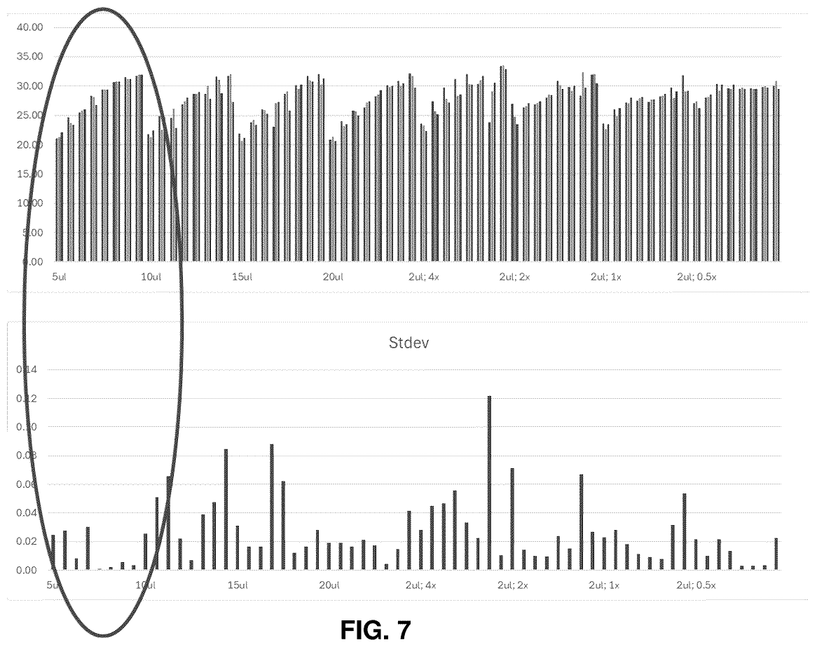

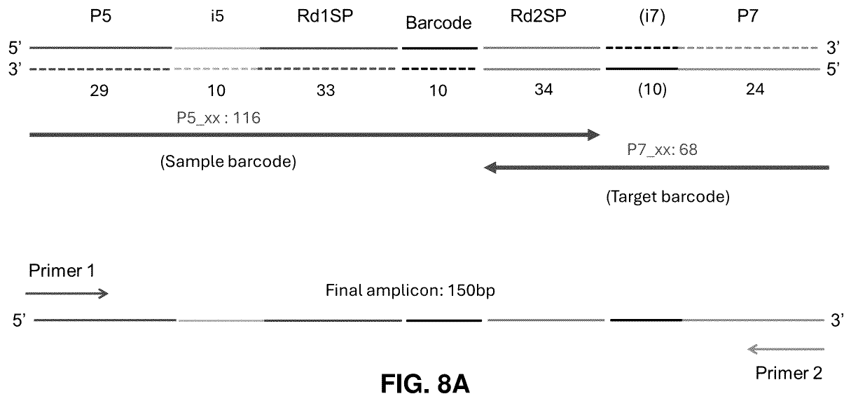

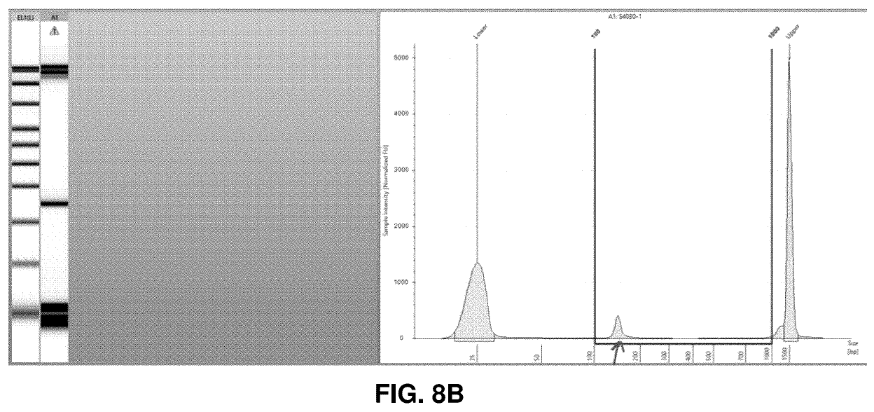

Reactions in 10 ul volume (5 ul sample+5 ul bead mix) showed the better consistency and lowest CV (FIG. 7).

### VIII. Eluting the Complex

The antibodies and antigens bound to the magnetic beads were denatured and inactivated by using NaOH solutions of different concentrations, and then detached from the magnetic beads. The NaOH treatment condition was ten minutes at room temperature. The magnetic beads were then washed with PBST to remove the supernatant and resuspended to the initial volume. The biotin-labeled antibodies remaining on the magnetic beads were detected using fluorescently labeled secondary antibodies. As the NaOH concentration increased, the less biotin-labeled antibodies remained on the magnetic beads under the same treatment conditions, which meant that the elution efficiency of the complex was higher.

### Example 2. BIQ-ELISA Experiment

1. Dilute the fully resuspended antibody coated magnetic beads and antibody oligonucleotide conjugates in the detection diluent in appropriate proportions and mix thoroughly. Use a pipette to add the mixed reaction solution into a 96 well plate with a V-bottom, with 5 μL per well.

2. Add corresponding testing standards or samples to each well, with 5 μL per well.

3. Use sealing film to seal the reaction hole. Centrifuge at 500×g for 1 minute to allow the sample, magnetic beads, and antibody oligonucleotide conjugate to converge at the bottom of the reaction well.

4 Incubate the reaction plate at room temperature at a speed of 700 revolutions per minute for 150 minutes.

5. After incubation, centrifuge at 500×g for 1 minute to allow all liquids to converge at the bottom of the reaction well, and carefully remove the sealing film.

6. Add 40 μL of detection diluent to each reaction well to increase the volume by 50 μL.

7. Place the reaction plate on the magnetic rack and let it stand for 2 minutes to ensure that the magnetic beads are fully stable. Transfer the reaction plate and magnetic frame to a fully automatic washing machine, and wash the reaction wells 5 times with washing buffer (phosphate buffer containing 0.1% Tween-20).

8. Remove the reaction plate from the magnetic rack and add 40 μL of elution buffer (10 mM sodium hydroxide) to each reaction well. Use a fully automatic washing machine or pipette to blow the magnetic beads thoroughly with the elution buffer, and then let them stand at room temperature for 10 minutes to release oligonucleotides from the magnetic beads.

9. Place the reaction plate back on the magnetic rack and let it stand for 2 minutes. Use a pipette to transfer 5 μL of supernatant containing oligonucleotides from each well to a clean PCR plate.

10. Add 15 μL of PCR primer premix (10 μL 2× Luna @ universal qPCR premix) to the PCR well containing the supernatant, 5 μL of water and 25 nM upstream and downstream primers were used to seal the PCR wells with a sealing membrane.

11. Centrifuge at 11.500×g for 1 minute to allow all liquids to converge at the bottom of the PCR well. Place the PCR plate into the qPCR instrument for reaction and record the signal value changes of SYBR Green

12. Draw a standard curve based on the Ct value generated by the standard sample and the concentration of the standard sample, and calculate the concentration of the target protein in the original sample using the Ct value and dilution factor generated by the position sample.

### Example 3. Comparison of SIMOA Technology and BIQ ELISA for Detecting TNFa

TABLE 5
Limit of Detection (LOD) sensitivity of five targets

| (pg/ml) | BIQ-ELISA | ELISA | SIMOA |
|---|---|---|---|
| IL-6 | 0.03 | 3 | 0.05 |
| IFNg | 0.015 | 15 | 0.03 |
| TNFa | 0.025 | 30 | 0.01 |
| IL-1b | 0.006 | 0.3 | 0.01 |
| IL-12p70 | 0.012 | 1 | 0.005 |

TABLE 6
Patient serum sample testing of TNFa

| TNFa | SIMOA | BIQ |
|---|---|---|
| SA1 | 2.90 | 28.41 |
| SA2 | 1.69 | 71.04 |
| SA3 | — | 6.46 |
| SA4 | — | 1.14 |
| SA5 | — | 1.62 |
| SA6 | 170.81 | 204.30 |
| SA7 | — | 3.26 |
| SA8 | 105.83 | 164.12 |
| SA9 | — | 20.56 |
| SA10 | 91.60 | 151.84 |
| SA11 | 242.94 | 316.42 |
| SA12 | 2.39 | 79.60 |

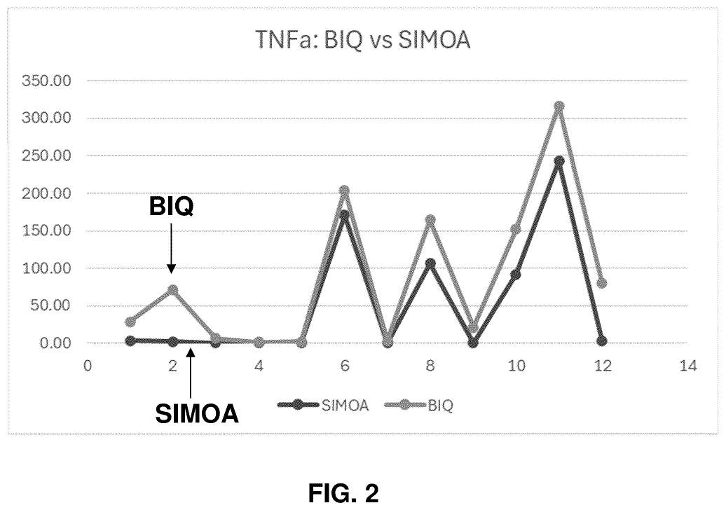

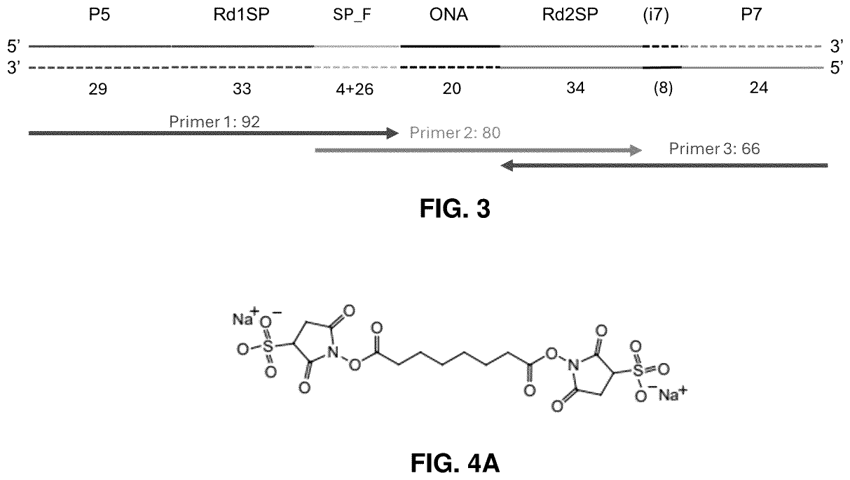

A plot comparing Single Molecule Array (SIMOA) technology with BIQ ELISA for detecting TNFa is shown in FIG. 2.

### Example 4. RayBio® Human IL-6 BIQ-ELISA Kit Operating Procedure

The immunoquantitative enzyme-linked immunosorbent assay (BIQ-ELISA) based on RayBio Beam combines the specificity of sandwich ELISA, the sensitivity of real-time PCR, and the ease of operation of microspheres, making it a distinct protein detection method. In this system, the detection antibody (DAB) is conjugated with a unique oligonucleotide (ONA) barcode (DAB-ONA) for signal amplification, while the capture antibody (CAB) is labeled with biotin and pre absorbed onto streptavidin coated beads (CAB biotin SABeads). The whole process is simple and clear, requiring a total of four hours of laboratory testing time.

Step 1: Formation and purification of immune complexes (ONA-DAB antigen CAB biotin SA beads). The sample was incubated with DAB-ONA and CAB biotin SA beads in a filter plate. In the presence of the target protein, DAB-ONA binds to the target and attaches to CAB biotin SA beads through biotin streptavidin interaction, and the unbounded protein is removed by washing.

Step 2: PCR amplification. The bound immune complexes will be released from the beads and transferred as templates for qPCR. Add the primer and PCR main mixture to the well and collect data using qPCR. Then use the Ct values obtained from qPCR to calculate the amount of antigen contained in each sample, where lower Ct values indicate higher concentrations of antigen. In a real experiment, a set of target protein standards with known concentrations will be run simultaneously to generate a standard curve for quantifying unknown samples.

### Reagent and Sample Preparation

1. Simply centrifuge the primer and probe mixing tube, and then recombine the powder with 0.55 micronuclease free water. Dilute the dilution solution twice with deionized water or distilled water before use. Suggest diluting normal serum/plasma 2-10 times. Note: The target protein levels may vary among different specimens. Researchers must determine the optimal dilution factor for each sample. Prepare 1 wash buffer solution I and 1 wash buffer solution

II. If there are visible crystals in the 20 fold washing solution, heat to room temperature and gently stir until dissolved. Dilute 15 mL of 20× Wash buffer into deionized water or distilled water to obtain 300 mL of 1× Wash buffer. Dilute 0.5 ml of 10× elution buffer into 4.5 ml of distilled water to obtain 5 ml of 1× elution buffer. Simply rotate the oligonucleotide binding antibody vial before resuspension. Measure the resuspended antibody in the dilution solution at 500 μl\hat{ }1. Preparation of human interleukin-6 standard: Briefly describe the human interleukin-6 standard bottle. Add 1 ml of prepared 1× dilution solution to prepare a standard solution of 0.4 ng/mL. Gently stir the powder until it completely dissolves. Pipette 160 μl of diluted solution for measurement and place it into 7 additional test tubes. Produce dilution series using 0.4 ng/mL standard solution (as shown below). Thoroughly mix each tube before the next transmission. 1× diluted solution was used as a blank control.

### Procedure

1. Before use, place all reagents and samples at room temperature (18-25° C.). The PCR main mixture, primer and probe mixture should be kept on ice after thawing. Suggest running all standards and samples in duplicate or triplicate.

2. According to your experiment, label the filter plate appropriately.

3. Add 200 μl of diluent to each well for pre wetting, and incubate at room temperature for 5 minutes. Choose one of the following methods based on the available devices. Vacuum manifold: Open the vacuum, open the valve, and place the filter plate on top of the vacuum. Pressure exceeding 10 inches of mercury (254 millimeters of mercury) is not allowed. After the well drainage is completed, close the valve and remove the valve plate. Use absorbent paper to dry the bottom of the tray and remove any residual liquid. Centrifuge: Stack the filter plates on top of the 96 well flat bottom plate and mark them as washing plates. Ensure that the well is aligned. Centrifuge the washing dish at 500 g for 1 minute, then carefully discard the effluent and dry clean the dish with absorbent paper for future use.

4. Add 5 μl of oligonucleotide binding antibody to a small bottle of beads coated with human IL-6 antibody. Rotate the small bottle for 30 seconds to thoroughly mix the various ingredients. 25 μl aliquots per well. Attention: This money is enough to drill 48 wells.

5. Add 25 μl of each standard sample to the appropriate well, making the total volume of each well 50 μl. Cover the lid. Place the steel plate on the eye socket steel plate vibrator. Shake at room temperature at a speed of 700 revolutions per minute for 2.5 hours.

6. As described in step 3.7, abandon the solution in each well. Add 200 μl of 1× Wash Buffer I to clean the plate and incubate at room temperature for 1 minute to remove the liquid as described in step 3.

7. Repeat the washing steps four more times, for a total of five washes. Attention: It is recommended to centrifuge the board at 500 g for 1 minute after the last cleaning, even with a vacuum manifold, to completely remove any liquid residue. This helps to reduce the deviation between wells.

8. Add 200 μl of 1× Wash Buffer II to clean the plate and incubate at room temperature for 1 minute to remove the liquid as described in step 5. Repeat the washing steps twice more, for a total of three washes.

9. To completely remove liquid residue, stack the filter plates on a 96 well flat bottomed plate labeled as a washing plate. Centrifuge the washing dish at 500 g for 1 minute, then discard the washing dish.

10. Add 25 μl of elution buffer to each well and incubate at room temperature for 10 minutes. In this step, complete step 12

11. Stack the filter plates on top of the 96 well flat bottom plate and mark them as collection plates. Ensure alignment.

12. Centrifuge with a 500 g centrifuge for 1 minute, then carefully separate the two plates.

Note: The oligonucleotide conjugated antibody antigen complex is currently in the collection tray and should not be discarded. Prepare a 1×PCR main mixture by mixing 2 volumes of 2×PCR main mixture with 1 volume of primer and probe mixture. Each plate hole requires 15 μl.

13. Transfer 5 μl of oligomeric antibody antigen complex from the collection plate (step 11) to the corresponding wells containing 15 μl of 1×PCR mixture in the PCR plate, so that the total volume in each well is 20 μl.

14. Thoroughly stir the wellbore with a pipette (at least 3 times up and down). Cover the sheet with the provided polymerase chain reaction plate film, ensuring that the film is evenly pressed onto the sheet and forming a sealing ring around each hole in the sheet.

15. Centrifuge the sealed polymerase chain reaction plate at 500 g for 1 minute and collect all liquids at the bottom of the well. Place the tablet into the real-time PCR instrument and perform the following detection settings using VIC compatible wavelengths.

### Laboratory Procedure

1. Prepare all reagents, samples, and standards as required.

2. Pre wet the oil well.

3. Add 25 μl of bead and conjugate mixture, as well as 25 μl of each standard and sample, to each well. Shake and incubate at room temperature for 2.5 hours.

4. Wash dishes.

5. Add 25 μl of elution buffer to each well. Cultivate at room temperature for 10 minutes. Collect displaced persons.

6. Perform real-time polymerase chain reaction.

### Result Calculation

The main data output of the BIQ-ELISA kit is Ct value. These values represent the number of cycles required for the sample to pass the fluorescence threshold. When DNA is amplified, additional fluorescent signals are generated, and each cycle leads to approximately doubling of DNA. Therefore, higher levels of DNA (directly related to the number of antigens in the sample) result in lower Ct values.

If present, remove obvious outliers from the results.

Calculate the average CT value of each set of three sets of standards, controls, and samples. Subtract the CT value of each sample from the control group to obtain the difference between the control group and the sample (Delta CT). Use logarithmic scale to plot standard values on the X-axis. This chart is the fastest way to visualize results, although not the most accurate. If this method is used, the concentration of unknown samples can be estimated using the best fit logarithmic line. The best fit line will have an equation y=k (x)+b, where Y is the Delta CT value and x is the concentration. To calculate the concentration of an unknown sample, you can input it into Excel in the following format:

Among them, Y is the Delta CT obtained during the measurement period, and B and K are obtained from the best fit line.

### Standard Curve

These standard curves are only for demonstration purposes. A standard curve must be run during each measurement

The minimum detectable concentration of human IL-6 is 0.03 μg/ml

The spiked recovery rate of human serum IL-6 is 105%, with a range of 94-126%. The recovery rate of cell culture medium is 101%, with a recovery rate between 93-112%.

Reproducibility internal analysis: Analysis within CV<10%: CV<15%

### Example 5: Characterizing Reaction Conditions

### 1) Characterize Buffer Solution

Different buffer solutions have a significant impact on the background of the experiment. Therefore, different buffer components are used to compare and select the combination with the lowest background.

Based on this result, mixing 10% casein, 5% BSA, and B1002 may be a better combination.

But in subsequent experiments, it was found that the presence of BSA caused many detection results to have a higher background. Therefore, in subsequent experiments, BSA was removed.

TABLE 7
Identification of suitable blocking buffers

| Blocking buffer | IL-6 antigen addition | Ct mean | Ct SD |
|---|---|---|---|
| 10C5B | Yes | 22.951 | 0.848 |
| 10C5B | No | 24.066 | 0.526 |
| CB1002 | Yes | 24.937 | 0.236 |
| CB1002 | No | 23.922 | 2.166 |
| 5% BSA + 0.1% Tween 20 | Yes | 23.298 | 1.534 |
| 5% BSA + 0.1% Tween 20 | No | 23.089 | 2.069 |
| Mixed Biolipidure | Yes | 24.123 | 0.921 |
| Mixed Biolipidure | No | 21.708 | 1.247 |

5% BSA and 0.1% Tween 20 in water
Biolipidure mix: in 4750 μl PBS, add 50 μl of B203, B206, B802, B804 and B1002, which are all 5% (w/v) solution
PBS containing 10% casein and 5% BSA (10C5B)
PBS containing 10% casein and 1% (w/v) B1002 (CB1002)

Use of blocking DNAs such as salmon sperm DNA, and short synthetic block DNA had no effect on the results.

### 2) Characterize the Conditions for Magnetic Automatic Washing

In order to compare different washing conditions and parameters, DNA sequences were directly coated on the surface of magnetic beads and could be directly amplified by PCR to compare Ct values.

In preliminary testing, the parameters required for the needle of the washing machine to be located in the middle of the hole were visually determined and further evaluated based on this.

TABLE 8
Comparison of Washing Conditions and Parameters for Magnetic Bead DNA Amplification

| | | | | | Final | Final | | |
|---|---|---|---|---|---|---|---|---|
| | Aspiration | Aspiration | Dispense | Dispense | aspiration | aspiration | | |
| Conditions | X | Z | X | Z | X | Z | Ct mean | Ct SD |
| 1 | 30 | 120 | −5 | 250 | 30 | 120 | 22.331 | 1.276 |
| 2 | 30 | 129 | −5 | 250 | 30 | 129 | 23.144 | 1.396 |
| 3 | 30 | 120 | 0 | 250 | 30 | 120 | 21.176 | 0.801 |
| 4 | 30 | 129 | 0 | 250 | 30 | 129 | 20.417 | 1.066 |
| 5 | 30 | 120 | 5 | 250 | 30 | 120 | 20.942 | 0.63 |
| 6 | 30 | 129 | 5 | 250 | 30 | 129 | 20.287 | 1.02 |
| 7 | 30 | 129 | 0 | 250 | 30 | 120 | 20.639 | 0.648 |
| | | | | | | Beads | 15.877 | 0.465 |
| | | | | | | control | | |

Based on this result, the parameters set for condition 6 appear to have the best overall effect. The travel rate of the needle used at this time is set to the default value of 3. According to the instructions, choosing a faster movement speed when washing magnetic beads will help retain them. A simple comparison was made regarding this.

TABLE 9
Comparison of wash conditions and travel rates on Ct values

| Conditions | Wash times | Travel rate | Ct mean | Ct SD |
|---|---|---|---|---|
| Beads control | 0 | — | 16.782 | 0.294 |
| Travel rate 3 | 5 | 3 | 19.031 | 0.764 |
| Travel rate 6CW | 5 | 6CW | 18.155 | 0.628 |

In each wash, if there is liquid residue, it will lead to a decrease in washing efficiency and result in a higher background. Therefore, compare the residual liquid situation under different travel rates of the washing machine needle movement.

For conditions with liquid residue, increase the delay time of the needle until the liquid can be completely absorbed.

TABLE 10
Effect of travel rate on liquid residue and drying time

| Travel | Rate | Liquid | Delay time to | Travel | Rate | Liquid | Delay time to |
|---|---|---|---|---|---|---|---|
| rate | (mm/s) | residue | completely dry | rate | (mm/s) | residue | completely dry |
| 1 | 4.1 &1 | Not | — | 1CW | 4.1 | Not | — |
| | | tested | | | | tested | |
| 2 | 5 & 1 | No | — | 2CW | 5 | No | — |
| 3 | 7.3 & 1 | No | — | 3CW | 7.3 | No | — |
| 4 | 9.4 & 1 | Slight | About 3 s | 4CW | 9.4 | Some | 1 s |
| 5 | 9.4 & 2 | Slight | 5 s | 6CW | 14.7 | A lot | 5 s |

Mix magnetic beads with a certain concentration of DNA solution, wash under different conditions, resuspend to the original volume, and PCR.

TABLE 11
Impact of travel rate and delay time on Ct values

| Travel rate | Delay/s | Ct mean | Ct SD | Travel rate | Delay/s | Ct mean | Ct SD |
|---|---|---|---|---|---|---|---|
| Positive control | — | 11.928 | 0.147 | Negative control | — | 29.178 | 0.079 |
| 3 | 0 | 19.298 | 2.003 | 3CW | 0 | 26.656 | 2.85 |
| 4 | 3 | 27.036 | 2.974 | 4CW | 3 | 24.475 | 4.955 |
| 5 | 5 | 27.562 | 2.753 | 6CW | 5 | 28.371 | 0.548 |

Considering the loss of magnetic beads and washing background, the optimal conditions are a residence time of 6CW and 5s.

3) Characterize the Raw Materials and Reaction Conditions for qPCR

Due to the presence of NaOH in the supernatant of antibodies labeled with eluted DNA, it may affect the subsequent PCR reaction. Compare the concentration and volume of NaOH solution that PCR reaction can withstand.

Mix 2×PCR premix containing the same concentration of DNA and primers with NaOH solution of different concentrations in equal volumes, and perform PCR to compare the Ct value changes.

TABLE 12
Effect of NAOH concentration on
pH, sodium ion levels, and Ct values

| NaOH | | pH after | Additional Na+ | | |
|---|---|---|---|---|---|
| Concen- | | mixing | concentration | | |
| tration/ | | with 2 × | in the | Ct | Ct |
| mM | pH | buffer | final mix/mM | value | mean |
| 100 | 13 | >10 | 50 | — | — |
| 10 | 12 | About 9.0 | 5 | 23.018 | 3.222 |
| 7.5 | 11.88 | — | 3.75 | 13.899 | 0.452 |
| 5 | 11.67 | About 8.5 | 2.5 | 11.938 | 0.729 |
| 2.5 | 11.40 | About 8.5 | 1.25 | 11.751 | 0.897 |
| 1 | 11 | About 8.5 | 0.5 | 11.339 | 0.398 |
| 0 | 7 | About 8.5 | 0 | 12.669 | 1.102 |

According to this result, when the concentration of NaOH solution is less than 7.5 mM during equal volume mixing, the PCR reaction is not affected. However, considering that the elution efficiency of the complex under these conditions may be low, reducing the volume of supernatant used for PCR, but using a higher concentration of NaOH can both increase the initial template content and ensure that PCR is not affected. Therefore, 10 mM NaOH was chosen to elute the complex, and half of the volume of 2×PCR premix was used for detection.

In this PCR detection system, due to the use of SYBR Green dye, the detection background is caused by non-specific primer dimers. In order to reduce the generation of primer dimers, thereby lowering the background and improving sensitivity, different primer concentrations were used in PCR reactions to compare their backgrounds.

TABLE 13
Effect of primer concentration on Ct values

| Primer concentration | 50 nM | Ct SD | 45 nM | Ct SD | 40 nM | Ct SD | 25 nM | Ct SD | 12.5 nM | Ct SD |
|---|---|---|---|---|---|---|---|---|---|---|
| Positive sample | 16.815 | 0.243 | 17.382 | 0.062 | 16.913 | 0.158 | 21.671 | 0.021 | 29.156 | 0.122 |
| Negative sample | 25.988 | 0.723 | 26.609 | 0.328 | 26.483 | 2.545 | 39.754 | 0.147 | — | — |
| Delta Ct | 9.173 | | 9.227 | | 9.57 | | 18.083 | | — | |

TABLE 14
Effect of primer concentration on Ct values for IL-1β detection

| Primer concentration | 35 nM | Ct SD | 30 nM | Ct SD | 20 nM | Ct SD | 15 nM | Ct SD |
|---|---|---|---|---|---|---|---|---|
| Sample of 100 pg/ml | 18.164 | 0.094 | 19.51 | 0.05 | 26.906 | 0.14 | 28.740 | 0.068 |
| IL-1β | | | | | | | | |
| Sample of 0 IL-1β | 30.216 | 0.055 | 32.311 | 0.159 | — | — | — | — |
| Delta Ct | 12.055 | | 12.81 | | — | | — | |

Based on this result, when the primer concentration is around 25 nM, the background of the PCR reaction is very small. Although the Ct value of the positive sample also increases, overall, its delta Ct range becomes larger, which helps to improve the sensitivity and discrimination of the reaction.

### 4) Adjusting the Concentration Ratio of Different Reaction Components

Based on previous experience, the magnetic bead suspension and sample are mixed in a medium volume for detection. In the initial reaction design, the magnetic beads and sample volume used were both 25 μL, and the final reaction volume was 50 μL. To reduce the sample size, try using smaller volumes of samples and magnetic beads. However, in order for the system to fully contact the ring-shaped magnet, a volume of approximately 40 μL is required.

Compare the changes in Ct values of the same sample by mixing, incubating, washing, and PCR testing with different volumes and the same mixture of magnetic beads and DNA conjugated antibody complexes.

TABLE 15
Comparison of Ct Values for IL-6 Detection

| IL-6 concentration | 50 μl system | Ct | 40 μl system | Ct |
|---|---|---|---|---|
| (pg/ml) | Ct mean | SD | Ct mean | SD |
| 400 | 19.372 | 0.406 | 18.804 | 0.794 |
| 133.33 | 20.775 | 0.279 | 20.388 | 1.189 |
| 44.44 | 22.645 | 0.596 | 22.068 | 0.137 |
| 14.81 | 23.874 | 0.937 | 23.583 | 0.303 |
| 4.94 | 25.270 | 0.404 | 25.376 | 0.679 |
| 1.65 | 26.987 | 0.981 | 26.525 | 0.506 |
| 0.55 | 29.027 | 0.729 | 28.726 | 0.777 |
| 0 | 32.303 | 0.092 | 32.465 | 0.249 |

The volume of the system is further reduced.

According to this result, using a reaction volume of 10 μL does not result in significant changes in Ct values. But this can greatly save reagents and samples, so this condition is chosen.

TABLE 16
Effect of reaction system volume on Ct values for IL-6 detection.

| IL-6 final | | | | | | | | |
|---|---|---|---|---|---|---|---|---|
| concentration | 10 μl system | Ct | 20 μl system | Ct | 30 μl system | Ct | 40 μl system | Ct |
| (pg/ml) | Ct mean | SD | Ct mean | SD | Ct mean | SD | Ct mean | SD |
| 200 | 21.484 | 0.528 | 21.515 | 0.334 | 21.210 | 0.656 | 20.953 | 0.400 |
| 66.67 | 23.941 | 0.658 | 23.672 | 1.601 | 23.824 | 0.393 | 23.585 | 0.449 |
| 22.22 | 25.761 | 0.216 | 25.325 | 1.074 | 25.722 | 0.430 | 25.488 | 0.423 |
| 7.41 | 27.759 | 0.852 | 27.128 | 0.430 | 25.815 | 2.387 | 26.955 | 0.577 |
| 2.47 | 29.387 | 0.033 | 28.647 | 0.046 | 27.890 | 1.780 | 28.693 | 0.495 |
| 0.82 | 30.732 | 0.068 | 29.351 | 0.991 | 29.963 | 0.371 | 29.995 | 0.126 |
| 0.27 | 31.313 | 0.172 | 31.312 | 0.396 | 31.147 | 0.508 | 30.453 | 0.447 |
| 0 | 31.884 | 0.119 | 31.870 | 0.189 | 31.173 | 0.883 | 31.207 | 1.318 |

### Primer & Probe Design for Multiplexing TaqMan Assays

TABLE 17
Five unique DNA barcodes

| Barcode Name | |
|---|---|
| (Target) | Barcode Sequence |
| Bp1 (IL-1b) | AGCACACATTGCTGGTTCTGCTCAC |
| | GTCTGACTACAGGCATTCCGTCTGT |
| | CTCTTATACACATCTCCGAGCCCAC |
| | GAGAC |
| | (SEQ ID NO: 1). |
| Bp3 (IL-6) | AGCACACATTGCTGGTTCTGCTCAC |
| | GTCAACCTAGTGCGACGATACCTGT |
| | CTCTTATACACATCTCCGAGCCCAC |
| | GAGAC |
| | (SEQ ID NO: 2). |
| Bp5 (IL-12p70) | AGCACACATTGCTGGTTCTGCTCAC |
| | GCCGAGGTCTGTCACAGTGTACTGT |
| | CTCTTATACACATCTCCGAGCCCAC |
| | GAGAC |
| | (SEQ ID NO: 3). |
| Bp7 (IFNg) | AGCACACATTGCTGGTTCTGCTCAC |
| | GGTGAACCGTATCTGACGGCACTGT |
| | CTCTTATACACATCTCCGAGCCCAC |
| | GAGAC |
| | (SEQ ID NO: 4). |
| Bp9 (TNFa) | AGCACACATTGCTGGTTCTGCTCAC |
| | GTCCCGTTGTATTAGCCGCCGCTGT |
| | CTCTTATACACATCTCCGAGCCCAC |
| | GAGAC |
| | (SEQ ID NO: 5). |

TABLE 18
Five matching TaqMan Probes

| Probe Name | TaqMan Probe Sequence |
|---|---|
| Tp1 | FAM-5′-CACGTCTGACTACAGGCATTCCGT |
| | (SEQ ID NO: 6). |
| Tp3 | VIC-5′-CACGTCAACCTAGTGCGACGATAC |
| | (SEQ ID NO: 7). |
| Tp5 | ABY-5′-CACGCCGAGGTCTGTCACAGTGTA |
| | (SEQ ID NO: 8). |
| Tp7 | JUN-5′-CACGGTGAACCGTATCTGACGGCA |
| | (SEQ ID NO: 9). |
| Tp9 | CY5-5′-CACGTCCCGTTGTATTAGCCGCCG |
| | (SEQ ID NO: 10). |

TABLE 19
Sequencing Primers

| Sequencing | |
|---|---|
| Primers | Primer Sequence |
| Forward_P | AGCACACATTGCTGGTTCTGCTCACG |
| | (SEQ ID NO: 11). |
| Reverse_R | GTCTCGTGGGCTCGGAGATG |
| | (SEQ ID NO: 12). |

An exemplary complete barcode sequence would be (29+33+30+20+34+8+24=178 bases)

(SEQ ID NO: 13)
5′-AATGATACGGCGACCACCGAGATCTACAC-
TCGTCGGCAGCGTCAGATGTGTATAAGAGACAG-
NNNNAGCACACATTGCTGGTTCTGCTCACG-
yyyyyyyyyyyyyyyyyyyy-CTGTCTCTTAT
ACACATCTCCGAGCCCACGAGAC-xxxxxxxx-
ATCTCGTATGCCGTCTTCTGCTTG-3′

Primer 1:(29+33+30-92 bp)

(SEQ ID NO: 14)
5′-AATGATACGGCGACCACCGAGATCTACAC-
TCGTCGGCAGCGTCAGATGTGTATAAGAGACAG-
NNNNAGCACACATTGCTGGTTCTGCTCACG

Primer 2: (26+20+34=80 bases)

(SEQ ID NO: 15)
5′-AGCACACATTGCTGGTTCTGCTCAC
G-yyyyyyyyyyyyyyyyyyyy-CTGTC
TCTTATACACATCTCCGAGCCCACGAGAC

Primer 3: (34+8+24=66 bases)

CTGTCTCTTATACACATCTCCGAGCCCACGAGAC-XXXXXXXX-
ATCTCGTATGCCGTCTTCTGCTTG-3′
(SEQ ID NO: 16)
Sequencing primers:
AGCACACATTGCTGGTTCTGC
(SEQ ID NO: 17)
(21 bp) & RD2SP
(GTCTCGTGGGCTCGGAGATG 20 bp) (SEQ ID NO: 12):
PCR Product 80 bp
TaqMan probes:
CACG-yyyyyyyyyyyyyyyyyyyy (24 bp, Tm62)

### 5) Reaction Conditions and Concept for NGS

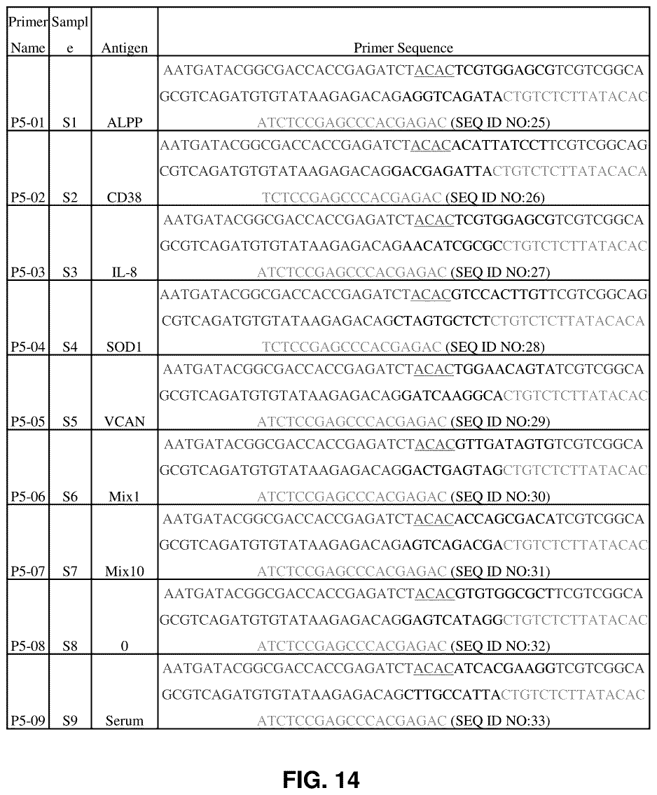

Five non-cross-reactive pairs were chosen to test the NGS approach. ALPP, CD38, IL-8, SOD1, VCAN pairs were individually validated and proved to have no cross reactivity. Their detection antibodies were barcoded with different DNA probes (FIG. 13) and were combined with the same ratio in reaction. Their biotinylated capture antibodies were coated on Streptavidin beads in their optimal dilution. Nine different samples were individually amplified using PCR with different sample index (FIG. 14). Equal volumes of the PCR products were combined for NGS. Samples 1-5 with individual recombinant antigen at 10 ng/ml. Sample 6 is the mixture of the five antigens at 10 ng/ml, and a further 10× dilution to make sample 7; sample 8 is a blank negative control, and sample 9 is 10× diluted normal serum sample. The NGS data were decoded by the NGS provider.

It was demonstrated that individual and multiplex have similar detection sensitivity and NGS can be successfully used for multiplex protein quantification (Tables 20-21).

TABLE 20
Sample Analysis of CAB-bio, Ag, and DAB-DNA Components

| Sample | CAB-bio | Ag | DAB-DNA |
|---|---|---|---|
| S1 | Mix | ALPP | Mix |
| S2 | Mix | CD38 | Mix |
| S3 | Mix | IL-8 | Mix |
| S4 | Mix | SOD1 | Mix |
| S5 | Mix | VCAN | Mix |
| S6 | Mix | Mix1 | Mix |
| S7 | Mix | Mix10 | Mix |
| S8 | Mix | 0 | Mix |
| S9 | Mix | Serum | Mix |

TABLE 21
NGS-based multiplex protein quantification
and detection sensitivity across targets

| Target | S1 | S2 | S3 | S4 | S5 | S6 | S7 | S8 | S9 |
|---|---|---|---|---|---|---|---|---|---|
| ALPP | 8281 | 6 | 17 | 13 | 7 | 8333 | 1378 | 0 | 176 |
| CD38 | 17 | 5790 | 218 | 163 | 190 | 5232 | 3419 | 7 | 5293 |
| IL-8 | 6 | 45 | 7360 | 143 | 19 | 6967 | 2667 | 3 | 9647 |
| SOD1 | 11 | 5 | 85 | 6545 | 30 | 6786 | 3763 | 3 | 7299 |
| VCAN | 7 | 31 | 65 | 92 | 4196 | 3318 | 1293 | 1 | 1881 |
| Total | 8322 | 5877 | 7745 | 6956 | 4442 | 28636 | 12520 | 14 | 24296 |
| reads | | | | | | | | | |

### Sequencing Primers

Forward primer:
(SEQ ID NO: 18)
5′-AATGATACGGCGACCACCGAGATCT-3′,
Tm = 62° C.
Reverse primer:
(SEQ ID NO: 19)
5′-CAAGCAGAAGACGGCATACGAGAT-3′,
Tm = 59° C.

### Protocol

Aliquot the diluted beads of the 5 targets individually in the PCR tubes
In assay diluent, dilute the human serum sample to the desired concentration.
Aliquot diluted serum samples into the PCR tubes then aliquot to the PCR tubes with corresponding beads
After incubation, use a magnetic stand to remove the supernatant.
Wash the beads with for 4 times with the magnetic stand
For each well, add combined conjugates in assay diluent and incubate for 2 hour with vertexing from time to time
Wash the beads with for 4 times with the magnetic stand
After the last wash, briefly centrifuge the strip again and then replace the strips back to the magnetic stand. Use pipette to remove the liquid.
Resuspend the beads in 20 μl nuclease water and transfer 5 μl into 10 μl 2×PCR master mix including 100 nM primers.
Add 5 μl water to each tube to bring the volume to 20 μl
Set PCR as following

TABLE 22

| Steps | Temperature/° C. | Time/s |
|---|---|---|
| Initial denature | 95 | 300 |
| 55 cycles | 95 | 15 |
| | 60 | 20 |
| Melt curve stage | 95 | 15 |
| | 68 | 60 |
| | 95 | 1 |

### Explore the Serum Levels of the Targets Selected

TABLE 23

| Sample | Serum Dilution factor | Ct mean | Ct SD |
|---|---|---|---|
| ALPP | 4 | 36.342 | 1.480 |
| CD38 | 4 | 31.976 | 0.234 |
| IL-8 | 4 | 32.695 | 3.355 |
| SOD1 | 500 | 42.503 | 0.416 |
| SOD1 | 1000 | 44.495 | 1.589 |
| VCAN | 10 | 39.373 | 1.574 |
| Negative control | — | 51.186 | 1.106 |

As the Ct values were desired to be similar level, the serum dilution factor is characterized as below.

TABLE 24

| Targets | Serum Dilution factor |
|---|---|
| ALPP | 4 |
| CD38 | 4 |
| IL-8 | 4 |
| SOD1 | 10 and 100 |
| VCAN | 4 |

### The DNA Barcode Used in the Assay

TABLE 25

| Sample number | P5 fragment | Antigen |
|---|---|---|
| 1 | P5-01 | ALPP |
| 2 | P5-02 | CD38 |
| 3 | P5-03 | IL-8 |
| 4 | P5-04 | SOD1 |
| 5 | P5-05 | VCAN |
| 6 | P5-06 | Combined undiluted |
| 7 | P5-07 | Combined 10-fold diluted |
| 8 | P5-08 | No antigen |
| 9 | P5-09 | Serum |

TABLE 26

| P7 fragment conjugated | Antigen |
|---|---|
| P7-01 | ALPP |
| P7-02 | CD38 |
| P7-03 | IL-8 |
| P7-04 | SOD1 |
| P7-05 | VCAN |

### Concentration of the Purified PCR Products

TABLE 27
Double strand DNA concentrations were measured by Qubit DsDNA high sensitivity assay kit.

| | | 10 | Combined | Combined | Combined | Combined | Combined |
|---|---|---|---|---|---|---|---|
| Samples | 0 | ng/μl | ALPP | CD38 | IL-8 | SOD1 | VCAN |
| Reading | 322 | 60922 | 764 | 917 | 1110 | 955 | 746 |
| Net reading | 0 | 60600 | 442 | 595 | 788 | 633 | 424 |
| Undiluted | | | 0.73 | 0.98 | 1.30 | 1.04 | 0.70 |
| concentration | | | | | | | |
| (ng/μl) | | | | | | | |

| | Combined | Combined | | | | | SOD1 | | SOD1 |
|---|---|---|---|---|---|---|---|---|---|
| Samples | mix 1 | mix 2 | Combined 0 | ALPP | CD38 | IL-8 | 10× | VCAN | 100× |
| Reading | 1130 | 793 | 296 | 597 | 637 | 1436 | 1221 | 593 | 472 |
| Net reading | 808 | 471 | −26 | 275 | 315 | 1114 | 899 | 271 | 150 |
| Undiluted | 1.33 | 0.78 | −0.04 | 0.45 | 0.52 | 1.84 | 1.48 | 0.45 | 0.25 |
| concentration | | | | | | | | | |
| (ng/μl) | | | | | | | | | |

Here the development of an magnetic Bead and Immune qPCR-based ELISA (BIQ-ELISA) platform for the ultra-sensitive protein quantification is disclosed. The BIQ-ELISA technology takes advantage of the specificity of sandwich-based ELISA, the sensitivity of real-time PCR, the easy manipulation of magnetic microspheres to lower the background, and integrates them into a single, easy-to-use platform. The BIQ-ELISA method can achieve femtogram per milliliter detection sensitivity, which is 1000 times more sensitive than traditional ELISA.

Unlike PLA and PEA, where both antibodies are tagged with DNA sequences that require proximity for PCR amplification, BIQ-ELISA uses only one DNA-barcoded antibody. The detection antibody (DAB) is conjugated with an oligonucleotide acid (ONA) barcode that allows for direct PCR amplification. This approach increases detection sensitivity without needing two DNA tags for proximity-based amplification, making the system more straightforward and potentially more robust. The capture antibody (CAB) is biotin-labeled and pre-adsorbed onto streptavidin-coated magnetic beads. These magnetic beads not only increase the surface area for antibody-antigen interactions, reduce reaction volume by allowing a homogeneous assay, simplify handling and washing, but also enhance sensitivity through a solid surface assay system. In summary, BIQ-ELISA simplifies the workflow compared to proximity-based methods like PLA and PEA, while still maintaining ultra-high sensitivity. This is accomplished by leveraging a single DNA-barcode for PCR amplification and the easy manipulation of magnetic microspheres to enhance the efficiency and sensitivity of the assay.

The procedure contains only two steps within a total of four hours of assay time. Step 1: The formation and purification of the immunocomplex (ONA-DAB-Antigen-CAB-biotin-SA-Beads). Samples are incubated with DAB-ONA and CAB-biotin-SA-Beads in a 96-well plate. In the presence of the target protein, the DAB-ONA binds to the target and attaches to the CAB-biotin-SA-Beads via the biotin-streptavidin interaction and the unbounded proteins are removed through washing with a magnetic auto washer. Step 2: PCR amplification. The bound immunocomplex will be released from the beads and transferred as template for qPCR. Primers and PCR master mix are added to the wells and data will be collected using qPCR. Ct values obtained from the qPCR are then used to calculate the amount of antigen contained in each sample, where lower Ct values indicate a higher concentration of antigen. The TaqMan assay and SYBR Green detection are two popular methods for real-time PCR. TaqMan assay uses sequence-specific probe to generate the fluorescence signal, while SYBR Green binds non-specifically to any double-stranded DNA. Since non-specific products like primer dimers or off-target amplicons may interfere with the SYBR Green assay, the TaqMan assay tends to have higher specificity and accuracy than SYBR Green Moreover, unlike SYBR Green, which is limited to detecting a single amplicon per reaction, TaqMan allows for the differentiation of multiple targets using unique fluorophore-labeled probes. TaqMan assays are widely used in molecular biology, primarily for quantitative PCR (qPCR) applications to detect and quantify nucleic acids. However, they are not traditionally used for direct protein detection. It was demonstrated that TaqMan technology can be successfully integrated into immuno-PCR for high sensitivity protein detection.

### BIQ-ELISA Advantages

Specificity: sandwich-based assay, ELISA standard
Sensitivity: PCR based detection; low background noise for bead-based washing
Simplicity: only two steps processing
Sample volume: homogeneous assay for low sample consumption
Automation: Efficiency & throughput, get data within 4 hours
Detection: qPCR or dPCR, No expensive equipment needed
Ultra sensitivity: up to 1000× more sensitive than traditional ELISA
Ultra-low sample volume: less than 10 ul
Multiplexing ready

Here the development of BIQ-ELISA technology including antibody DNA conjugation with the BS3 crosslinker; the use of biotinylated antibody absorption on streptavidin-coated magnetic beads; assay characterization; magnetic auto-washing setup; and the qPCR conditions for the assay are reported. With this system, at least five BIQ-ELISA kits for IL-6, IL-1b, IL-12p70, IFNg, and TNFa were developed. The standard curve test revealed that the detection sensitivity of all the five targets is comparable to SIMOA, being up to 1000× more sensitive than traditional ELISA. Testing a dozen patent serum samples for TNFa with both SIMOA and BIQ-ELISA showed that BIQ-ELISA detected TNFa in all samples, while SIMOA failed in five cases. The correlation between the two methods in calculating protein concentrations was 98%.

Overall, BIQ-ELISA matches SIMOA in detection sensitivity while offering significant advantages such as reduced sample volume, shorter assay time, higher throughput, and the ability to be performed with a regular qPCR machine. This makes BIQ-ELISA a promising approach for highly sensitive, multiplexed protein detection, especially for low-abundance protein biomarkers.

With the newly developed BIQ system, the TaqMan approach showed similar or even higher detection sensitivity than that of the regular qPCR with SYBR Green. In addition, multiplexing detection of 5 different targets were achieved by designing TaqMan probes with distinct fluorophores and pairing them with uniquely barcoded DNA-linked antibodies. In this system, the detection antibodies of the 5 different targets were uniquely DNA barcoded. Matching TaqMan probes with distinct fluorophores for the 5 DNA sequence were used in the qPCR assay. The study demonstrated that up to 5 different protein targets can be successfully multiplexed in a single assay, with the performance comparable to individual assays.

The integration of TaqMan technology into immuno-PCR represents an innovative approach for protein detection. The BIQ system leverages TaqMan's fluorescent probe detection to improve the accuracy and reliability of immuno-PCR. This approach maintains the high sensitivity of traditional immuno-PCR while introducing the advantage of multiplex analysis. This previously unidentified application of TaqMan technology expands its utility beyond traditional nucleic acid quantification, paving the way for advanced multiplex protein detection with significant implications in diagnostics and research.

### Example 6: Bead-Based Immuno-qPCR with Biotin-Labeled Samples (BIQL)

### Materials and Methods

### Detection of Recombinant Proteins

Ten recombinant proteins (MIP-1α, Eotaxin, IL-22, IL-12p40, IL-12p70, EXN4, IFN-γ, IL-1β, IL-6, and TNF-α) were biotinylated using EZ-Link NHS-PEG4-Biotin (Thermo Fisher, Cat #21362) following the manufacturer's protocol. To remove excess unreacted biotin, the biotinylated proteins were purified through 7K molecular weight cut-off desalting columns.

The biotinylated proteins were serially diluted and analyzed using both the BIQ and BIQL platforms. In the BIQ system, biotinylated proteins were first incubated with target-specific capture antibodies pre-immobilized on streptavidin-coated magnetic beads. After this binding step, DNA-conjugated detection antibodies were added to form the sandwich complex. Following incubation, unbound components were removed through multiple washes using a magnetic washer. The bound immunocomplex was then eluted with elution buffer, and its relative abundance was quantified by qPCR. In contrast, the BIQL system simplified the workflow by directly incubating biotinylated proteins with streptavidin-coated magnetic beads, eliminating the need for pre-immobilized capture antibodies. Subsequent steps, including incubation with DNA-conjugated detection antibodies, washing, elution, and qPCR detection, were identical to those used in the BIQ protocol. To ensure an accurate comparison between the two systems, identical amounts of magnetic beads and DNA-conjugated detection antibodies were used for the same targets.

### NGS Amplicon Library Construction

### DNA Barcode Design

The DNA barcodes used in the BIQL system were carefully designed to fulfill multiple requirements useful for both NGS-based detection and TaqMan assay compatibility, while reducing synthesis cost and maximizing assay efficiency. The design objectives included:
A unique DNA sequence for each target (see Table 28) to facilitate unambiguous target identification
A dedicated sequencing primer site for each barcode
Compatibility with sample barcoding, facilitating multiplex analysis across samples
The ability to be decoded by both next-generation sequencing (NGS) and TaqMan probe-based qPCR
Integration of all required Illumina sequencing components, including P5, i5, Read 1 Sequencing Primer (Rd1SP), Read 2 Sequencing Primer (Rd2SP), i7, and P7
Compact design, with all DNA fragments kept below 100 base pairs to reduce synthesis cost and improve efficiency

To satisfy these requirements, three distinct DNA fragments were designed:

Sequence 1 (Seq1: 92 bp) (SEQ ID NO:25):
Contained Illumina P5 (29 bp), i5 index (8 bp), and Read 1 Sequencing Primer (Rd1SP, 33 bp)
Included a 30 bp barcode sequencing primer site
The first 4 nucleotides were randomized to maximize sequencing diversity and authenticity

Sequence 2 (Seq2: 80 bp) was selected from Table 28:
Contained a unique 20 bp target-specific DNA barcode
Included a 30 bp barcode sequencing primer site
Contained the Read 2 Sequencing Primer (Rd2SP, 34 bp)
To enhance TaqMan assay efficiency, an additional 4 bp common sequence was added to the 3′ end of the sequencing primer region, creating a 24 bp TaqMan probe region
Sequence 2 was conjugated to the detection antibody, serving as the primary target identifier

TABLE 28
List of barcode sequences used for Sequence 2.

| | Barcode Sequence |
|---|---|
| Bp1 | AGCACACATTGCTGGTTCTGCTCAC |
| | GTCTGACTACAGGCATTCCGTCTGT |
| | CTCTTATACACATCTCCGAGCCCAC |
| | GAGAC |
| | (SEQ ID NO: 1) |
| Bp3 | AGCACACATTGCTGGTTCTGCTCAC |
| | GTCAACCTAGTGCGACGATACCTGT |
| | CTCTTATACACATCTCCGAGCCCAC |
| | GAGAC |
| | (SEQ ID NO: 2) |
| Bp5 | AGCACACATTGCTGGTTCTGCTCAC |
| | GCCGAGGTCTGTCACAGTGTACTGT |
| | CTCTTATACACATCTCCGAGCCCAC |
| | GAGAC |
| | (SEQ ID NO: 3) |
| Bp7 | AGCACACATTGCTGGTTCTGCTCAC |
| | GGTGAACCGTATCTGACGGCACTGT |
| | CTCTTATACACATCTCCGAGCCCAC |
| | GAGAC |
| | (SEQ ID NO: 4) |
| Bp8 | AGCACACATTGCTGGTTCTGCTCACG |
| | TATCGCCCAAGAGCGCAGTCCTGTC |
| | TCTTATACACATCTCCGAGCCCACG |
| | AGAC |
| | (SEQ ID NO: 34) |
| Bp9 | AGCACACATTGCTGGTTCTGCTCAC |
| | GTCCCGTTGTATTAGCCGCCGCTGT |
| | CTCTTATACACATCTCCGAGCCCAC |
| | GAGAC |
| | (SEQ ID NO: 5) |
| Bp10 | AGCACACATTGCTGGTTCTGCTCAC |
| | GACGCAGGATCTAAGCCAGCGCTGT |
| | CTCTTATACACATCTCCGAGCCCAC |
| | GAGAC |
| | (SEQ ID NO: 35) |

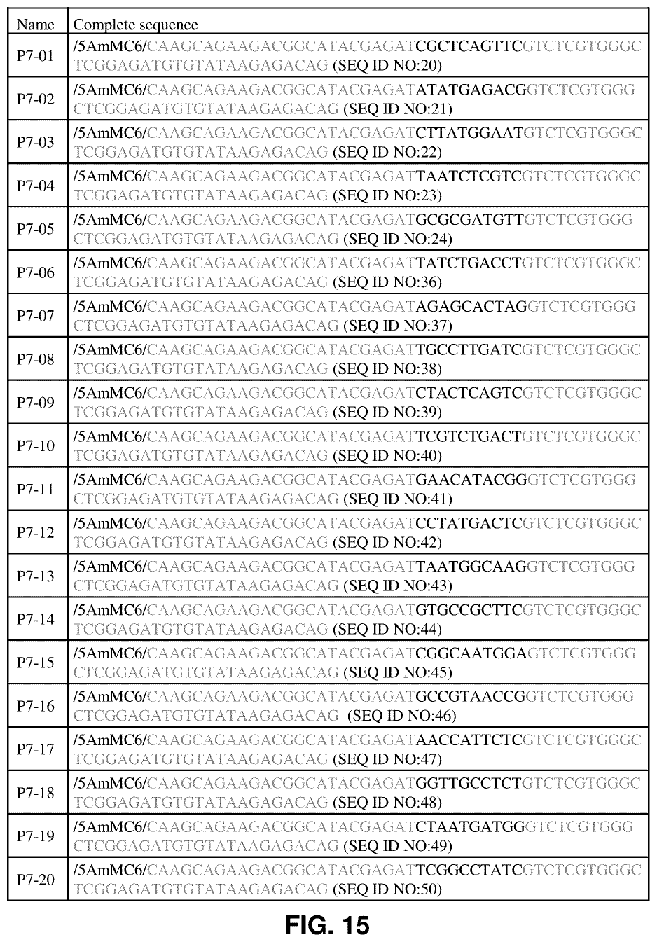

Sequence 3 (Seq3: 66 bp) was selected from the table in FIG. 15:
Included additional Illumina sequencing components: Read 2 Sequencing Primer (Rd2SP, 34 bp), i7 index (8 bp, used for sample indexing), and P7 adapter (24 bp).

In the BIQL workflow, Sequence 2 (attached to the detection antibody) carries the unique barcode for each target. This barcode can be read out either by NGS, using its dedicated sequencing primers, or by TaqMan assay, using its unique 24 bp probe region. When paired with Sequence 3, the i7 index provides sample-specific identification for NGS, greatly expanding multiplexing capacity. For example, combining 200 unique target barcodes with 100 unique i7 sample indexes results in a theoretical diversity of 20,000 unique detection events.

TABLE 29
List of matching TaqMan probes.

| | Matching TaqMan Probe |
|---|---|
| Tp1 | FAM-5′-CACGTCTGACTACAGGCATTCCGT |
| | (SEQ ID NO: 6) |
| Tp3 | VIC-5′-CACGTCAACCTAGTGCGACGATAC |
| | (SEQ ID NO: 7) |
| Tp5 | ABY-5′-CACGCCGAGGTCTGTCACAGTGTA |
| | (SEQ ID NO: 8) |
| Tp7 | JUN-5′-CACGGTGAACCGTATCTGACGGCA |
| | (SEQ ID NO: 9) |
| Tp8 | JUN-5′-CACGTATCGCCCAAGAGCGCAGTC |
| | (SEQ ID NO: 51) |
| Tp9 | CY5-5′-CACGTCCCGTTGTATTAGCCGCCG |
| | (SEQ ID NO: 10) |
| Tp10 | CY5-5′-CACGACGCAGGATCTAAGCCAGCG |
| | (SEQ ID NO: 52) |

TABLE 30
List of sequencing primers.

| Primers | Primer Sequence |
|---|---|
| SP2 | AGCACACATTGCTGGTTCTGCTCACG |
| | (SEQ ID NO: 11) |
| SP5 | AATGATACGGCGACCACCGAGATCTACAC |
| | (SEQ ID NO: 53) |
| SP7 | CAAGCAGAAGACGGCATACGAGAT |
| | (SEQ ID NO: 19) |

Prior to NGS library preparation, Sequence 1 was added to complete the Illumina library structure. The inclusion of a unique i5 index within Sequence 1 provided an additional layer of multiplexing by serving as a batch identifier, further increasing diversity. With 200 unique target barcodes, 100 i7 sample indexes, and 5 i5 batch indexes, a total of 100,000 distinct detection events can be achieved within a single sequencing run. This compact, modular barcode design facilitates scalable, cost-efficient, and highly multiplexed protein detection in the BIQL system, compatible with both NGS and TaqMan-based workflows.

### Methods for NGS Amplicon Library Construction

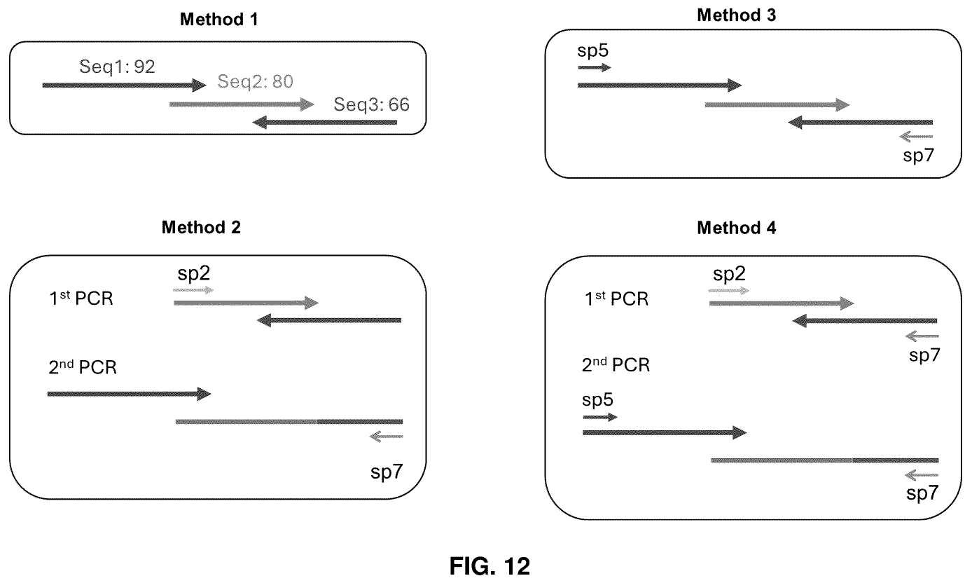

Four different methods were evaluated for constructing the NGS amplicon library using three DNA fragments (Sequence 1, Sequence 2, and Sequence 3) (FIG. 12).
Method 1: Direct PCR was performed by mixing all three DNA fragments. Sequence 1 and Sequence 3 were used as primers (Sequence 1 (Seq1: 92 bp) (SEQ ID NO:25); Sequence 2 (Seq2: 80 bp) selected from Table 28; Sequence 3 (Seq3: 66 bp) selected from the table in FIG. 15).
Method 2: A two-step PCR approach. In the first round, Sequence 2 and Sequence 3 were amplified using the SP2 primer. The PCR product was then purified, followed by a second round PCR using Sequence 1 and the SP7 amplification primer.
Method 3: Direct PCR using all three fragments, with specific SP5 and SP7 primers.
Method 4: A two-step PCR method. In the first round, Sequence 2 and Sequence 3 were amplified with SP2 and SP7 primers. The PCR product was purified using a PCR purification kit. In the second round, Sequence 1 was added to the purified product and amplified using SP5 and SP7 primers.

All four methods successfully produced the desired 170 bp amplicon. However:
Method 1 and Method 2 resulted in high levels of nonspecific background, likely due to the use of longer DNA templates as primers, which are less specific than short primers.
Method 3 yielded lower background, but the overall product yield was limited.
Method 4 provided both high yield and clean PCR product. Additionally, introducing Sequence 1 at the pre-NGS step, rather than separately, helped reduce sample-to-sample variation.

Method 4 was selected for further characterization. Final conditions were:
Template concentration: 1 nM each of Sequence 1 and Sequence 3
Primer concentrations:
   1st round PCR: 30 nM SP2 and SP7
   2nd round PCR: 20 nM SP5 and SP7

This protocol facilitated high-quality and consistent NGS library preparation.

To this end, five cytokines (IFN-γ, IL-1β, IL-6, IL-12p70, and TNF-α) were selected. These proteins were tested in both singleplex and multiplex formats using varying concentrations of biotinylated protein standards. Serial dilutions of the biotinylated serum were amplified using primers incorporating different i7 indices (with Sequence 3), running 25 PCR cycles to ensure amplification during the exponential phase (early Ct).

After PCR product purification, 3 μL from each reaction was pooled for a second round of PCR using primers containing Sequence 1, with 30 additional cycles. Final libraries were evaluated on SDS-PAGE gels, and concentrations were quantified using Qubit fluorometry. The pooled libraries were sequenced using an Illumina platform. Data were demultiplexed based on i7 indices and further confirmed using the unique target-specific barcodes.

TABLE 31
Sample conditions for NGS experiment.

| Sample | Ag | Conjugates | P7 |
|---|---|---|---|
| S1 | IFNg 10000 | IFNg | P7-01 |
| S2 | IFNg 1000 | | P7-02 |
| S3 | IFNg 100 | | P7-03 |
| S4 | IFNg 10 | | P7-04 |
| S5 | IL-1b 1000 | IL-1b | P7-05 |
| S6 | IL-1b 100 | | P7-06 |
| S7 | IL-1b 10 | | P7-07 |
| S8 | IL-1b 1 | | P7-08 |
| S9 | IL-6 4000 | IL-6 | P7-09 |
| S10 | IL-12p70 1000 | IL-12p70 | P7-10 |
| S11 | TNFa 10000 | TNFa | P7-11 |
| S12 | Mix 1x | Mix | P7-12 |
| S13 | Mix 10x | Mix | P7-13 |
| S14 | Mix 100x | Mix | P7-14 |
| S15 | Mix 1000x | Mix | P7-15 |
| S16 | 0 | Mix | P7-16 |
| S17 | Bio-Ag Serum 20x | Mix | P7-17 |
| S18 | Bio-Ag Serum 200x | Mix | P7-18 |
| S19 | Bio-Ag Serum 2kx | Mix | P7-19 |
| S20 | Bio-Ag Serum 20kx | Mix | P7-20 |

### Results and Discussion

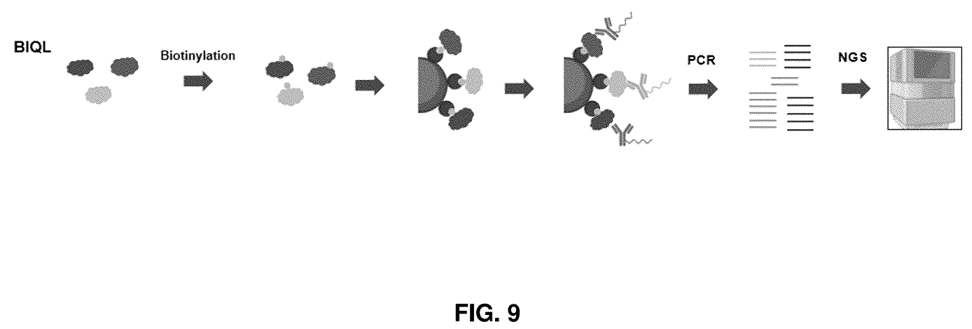

An advancement in high-throughput, ultra-sensitive protein quantification was established by integrating Bead-based Immuno-qPCR with biotin-labeled samples (BIQL) (FIG. 9). In this system, all proteins in the sample were first biotinylated, which resulted in universal labeling. These biotinylated proteins were immobilized onto streptavidin-coated magnetic beads and then incubated with an array of DNA-barcoded detection antibodies to form highly specific immunocomplexes. Unlike traditional sandwich assays that require a secondary antibody, this platform leveraged direct quantification via qPCR or next-generation sequencing (NGS), using the DNA tags as unique molecular identifiers. BIQL integrated the femtogram-level sensitivity of bead-based immuno-qPCR (BIQ) with the scalable multiplexing power of biotin-labeled sample arrays. By combining direct biotinylation of proteins, magnetic bead capture, and DNA-barcoded antibody detection, BIQL facilitated digital, highly specific quantification of up to 1000+ proteins per run—ideal for clinical biomarker discovery, systems biology, and next-generation diagnostics.

The development of the BIQL (Bead-based Immuno-qPCR with Labelled samples) technology, incorporated useful technical features that facilitated sensitive, high-throughput protein detection. The workflow included sample biotinylation, immobilization of biotinylated proteins onto streptavidin-coated magnetic beads, and antibody-DNA conjugation using the BS3 crosslinker. The system further featured exemplary assay conditions, rational DNA barcode design, and NGS amplicon library construction to facilitate multiplexed detection.

Using the BIQL platform, two protein panels covering both high- and low-abundance plasma proteins were evaluated. BIQL demonstrated detection sensitivity comparable to BIQ and SIMOA, while offering distinct advantages, including reduced sample volume requirements, shorter assay times, higher throughput, and the ability to perform up to 5-plex detection on a standard qPCR instrument. Additionally, BIQL signals were read out by next-generation sequencing (NGS), allowing scalable, ultra-sensitive, and highly multiplexed protein detection.

Importantly, by leveraging a plasma stratification strategy, where proteins are grouped based on their abundance range, BIQL holds the potential to allow the detection of thousands of proteins from a single drop of blood, making it a promising tool for biomarker discovery and clinical applications, particularly for low-abundance protein targets.

BIQL Exhibits Comparable or Superior Sensitivity to BIQ with Biotinylated Recombinant Proteins

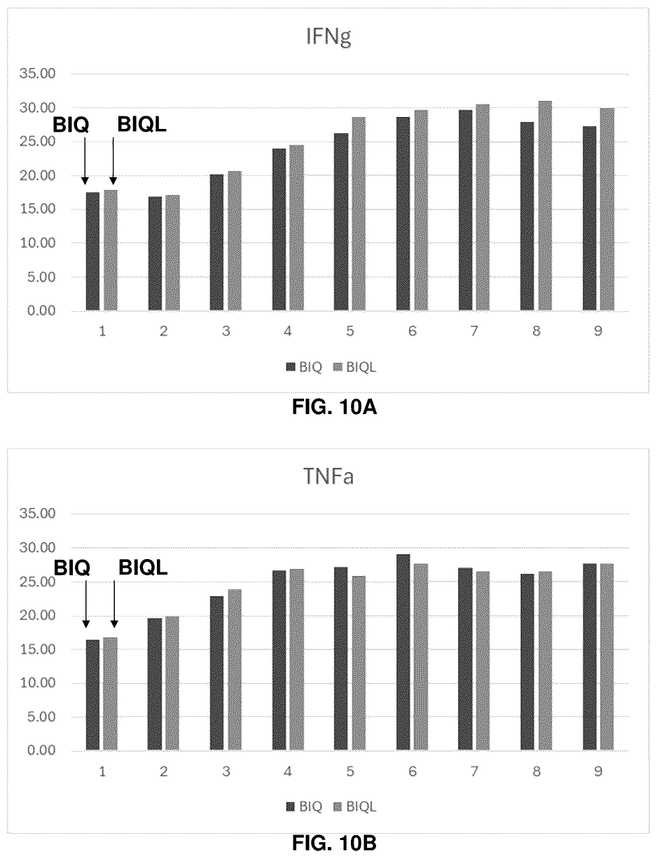

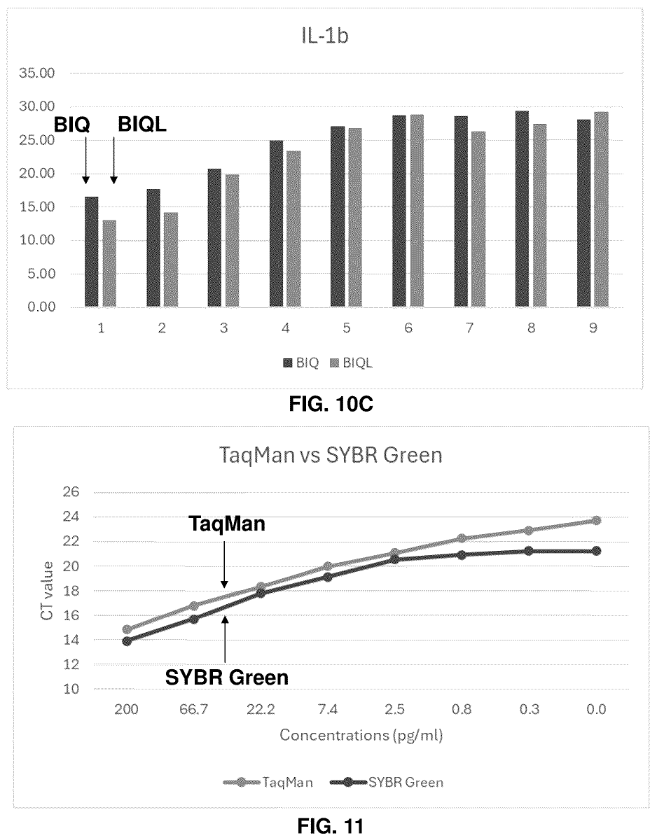

Across most targets, the cycle threshold (CT) values obtained from BIQL and BIQ were comparable, confirming similar detection sensitivity (FIGS. 10A-10C). Notably, IL-1β demonstrated significantly improved detection sensitivity in the BIQL assay compared to BIQ. This enhancement is likely due to reduced interference from suboptimal binding affinity between biotinylated IL-1β and its capture antibody, an issue that can compromise assay performance in conventional BIQ but is effectively mitigated in the BIQL system.

The detection sensitivity was determined based on standard curves generated using the BIQ kits. Among the five representative targets assessed, BIQL exhibited similar or superior sensitivity in comparison to BIQ, as summarized in Table 32. These results highlight the robustness and improved performance of BIQL, particularly for targets where antibody-antigen interactions may be compromised in traditional formats.

TABLE 32
Comparison of Detection Sensitivity
Between BIQ and BIQL for Selected Targets.

| | Detection | Detection | |
|---|---|---|---|
| Target | Sensitivity | Sensitivity | Performance |
| Protein | (BIQ) | (BIQL) | Comparison |
| IL-16 | 0.11 | 0.022 | Better |
| TNFa | 0.014 | 0.006 | Similar |
| IFNg | 0.07 | 0.04 | Similar |
| IL-6 | 0.03 | 0.05 | Similar |
| IL-1b | 0.006 | 0.001 | Better |

Note:
Detection sensitivity is expressed as the lowest detectable concentration (pg/mL) based on qPCR readouts.

### BIQL System Validation

Both HPX recombinant protein and one normal serum sample were biotinylated, and their concentrations were determined by the HPX BIQ kit. In the following experiment, the biotinylated HPX protein was then used as the standard in the BIQL system to determine the HPX concentration in the same biotinylated serum sample, as shown in Table 33. The calculated HPX concentration in the biotinylated serum were 360 μg/ml and 364 μg/ml for the BIQ and BIQL methods, respectively.

TABLE 33
BIQL system validation with HPX
recombinant protein and serum samples.

| Sample | BIQ | CT | BIQL | CT |
|---|---|---|---|---|
| Standard | 3000 | 16.82 | 4800 | 16.28 |
| | 1000 | 18.26 | 1600 | 17.64 |
| | 333.3 | 19.70 | 533.3 | 20.19 |
| | 111.1 | 21.42 | 177.8 | 22.18 |
| | 37.0 | 22.86 | 59.3 | 24.22 |
| | 12.3 | 24.55 | 19.8 | 25.28 |
| | 4.1 | 25.14 | 6.6 | 26.52 |
| | 0 | 26.71 | 0 | 27.42 |
| Bio-Serum | 400k | 18.46 | 400k | 20.27 |
| | 1600k | 20.42 | 1600k | 20.65 |
| | 6400k | 22.20 | 6400k | 23.96 |
| | 25600k | 24.20 | 25600k | 26.00 |
| | 25600k + Std2 | 19.24 | 25600k + Std2 | 19.19 |

### TaqMan Assay Offers Superior Sensitivity and Multiplexing Capability Compared to SYBR Green

TaqMan assays are widely used in molecular biology, primarily for quantitative PCR (qPCR) applications to detect and quantify nucleic acids. However, they are not traditionally used for direct protein detection. In Example 5, it was demonstrated that TaqMan technology can be successfully integrated into immuno-PCR for high sensitivity protein detection. The TaqMan assay and SYBR Green detection are two popular methods for real-time PCR. TaqMan assay uses sequence-specific probes to generate the fluorescence signal, while SYBR Green binds non-specifically to any double-stranded DNA. Since non-specific products like primer dimers or off-target amplicons may interfere with the SYBR Green assay, the TaqMan assay tends to have higher specificity and accuracy than SYBR Green.

To test this hypothesis, serially diluted biotinylated IL-6 samples were processed using the BIQL assay with two different detection chemistries: TaqMan probes and SYBR Green (FIG. 11). The CT values obtained from both systems demonstrated consistent behavior, showing a clear inverse correlation with IL-6 concentration. However, the SYBR Green assay exhibited higher background signals, resulting in lower apparent CT values for blank or low-concentration samples, consistent with its known susceptibility to non-specific amplification and primer-dimer artifacts. In contrast, the TaqMan assay maintained lower background and higher overall specificity. The calculated limit of detection (LOD) for TaqMan and SYBR Green are 0.005 μg/mL and 0.047 μg/mL respectively. These results confirmed that the TaqMan-based BIQL assay offers superior detection sensitivity compared to the conventional SYBR Green approach, further supporting the integration of TaqMan technology as a preferred method for highly sensitive protein detection within the BIQL platform.

Another key advantage of the TaqMan assay is its multiplexing capability. Unlike SYBR Green, which is limited to detecting a single amplicon per reaction due to its non-specific binding to all double-stranded DNA, the TaqMan platform allows simultaneous detection of multiple targets by using sequence-specific probes labeled with distinct fluorophores.

In this study, multiplex detection of five protein targets—IL-1β, IL-6, IL-12p70, IFN-γ, and TNF-α—was achieved by pairing uniquely DNA-barcoded detection antibodies with corresponding TaqMan probes, each labeled with a distinct fluorophore. Each antibody was conjugated to a unique DNA sequence, and a matching TaqMan probe was designed to hybridize specifically to that sequence during the qPCR assay. The multiplexed 5-plex assay was then run in parallel with the corresponding singleplex assays, using identical sets of standard protein concentrations for comparison. The results demonstrated that all five protein targets could be reliably detected in a single reaction, with performance metrics (sensitivity, dynamic range, and reproducibility) comparable to those of the individual assays, as summarized in Tables 34 and 35. This confirmed the robustness of TaqMan-based multiplexing within the BIQL system, offering a powerful tool for high-throughput, multi-analyte protein quantification from limited sample volumes.

The integration of TaqMan technology into immuno-PCR represents an advancement for protein detection. By leveraging TaqMan's sequence-specific, fluorescent probe detection, the BIQL system enhanced the accuracy, specificity, and reliability of traditional immuno-PCR while preserving its inherent high sensitivity. Moreover, the incorporation of TaqMan chemistry facilitates robust multiplex analysis, a significant improvement over conventional single-target assays. This application extends the utility of TaqMan technology beyond its traditional role in nucleic acid quantification, establishing a versatile platform for highly sensitive, multiplexed protein detection. The resulting BIQL-TaqMan system can be used for biomarker discovery, clinical diagnostics, and high-throughput protein analysis, addressing needs in both research and translational applications.

TABLE 34
Detection of protein targets using BIQL-TaqMan.

| TNFa | Single | Multiplex | IL-1b | Single | Multiplex | IL-6 | Single | Multiplex |
|---|---|---|---|---|---|---|---|---|
| 400 | 20.85 | 23.00 | 400 | 16.66 | 18.34 | 400 | 17.57 | 22.79 |
| 133.3 | 22.03 | 22.89 | 133.3 | 19.44 | 18.64 | 133.3 | 19.58 | 24.65 |
| 44.4 | 23.56 | 22.52 | 44.4 | 21.68 | 19.23 | 44.4 | 21.98 | 27.78 |
| 14.8 | 25.22 | 23.69 | 14.8 | 24.28 | 20.99 | 14.8 | 25.60 | 30.91 |
| 4.9 | 26.45 | 25.19 | 4.9 | 25.43 | 24.35 | 4.9 | 25.42 | 30.05 |
| 1.6 | 24.80 | 24.96 | 1.6 | 26.79 | 25.49 | 1.6 | 24.80 | 27.26 |
| 0.5 | 28.06 | 27.26 | 0.5 | 27.62 | 27.74 | 0.5 | 24.70 | 27.17 |
| 0 | 25.39 | 27.72 | 0 | 28.21 | 29.64 | 0 | 26.30 | 27.63 |

TABLE 35
Detection of protein targets using BIQL-TaqMan (continued).

| IFNg | Single | Multiplex | IL-12p70 | Single | Multiplex |
|---|---|---|---|---|---|
| 1000 | 22.88 | 21.94 | 1000 | 19.58 | 17.66 |
| 333.3 | 24.42 | 23.60 | 333.3 | 21.40 | 19.91 |
| 111.1 | 26.31 | 27.36 | 111.1 | 22.75 | 21.65 |
| 37.0 | 27.02 | 28.53 | 37.0 | 24.22 | 23.58 |
| 12.3 | 27.82 | 28.57 | 12.3 | 25.74 | 25.27 |
| 4.1 | 27.73 | 27.91 | 4.1 | 26.44 | 26.54 |
| 1.4 | 27.87 | 27.77 | 1.4 | 27.98 | 28.54 |
| 0 | 25.99 | 27.00 | 0 | 26.27 | 27.42 |

### NGS Results

The initial NGS run was designed to evaluate the feasibility of the system, including its specificity, sensitivity, multiplexing capability, and performance in serum samples. The NGS libraries were first demultiplexed using the i7 indices for the 20 samples and then decoded with the unique target-specific barcodes corresponding to the five cytokines.

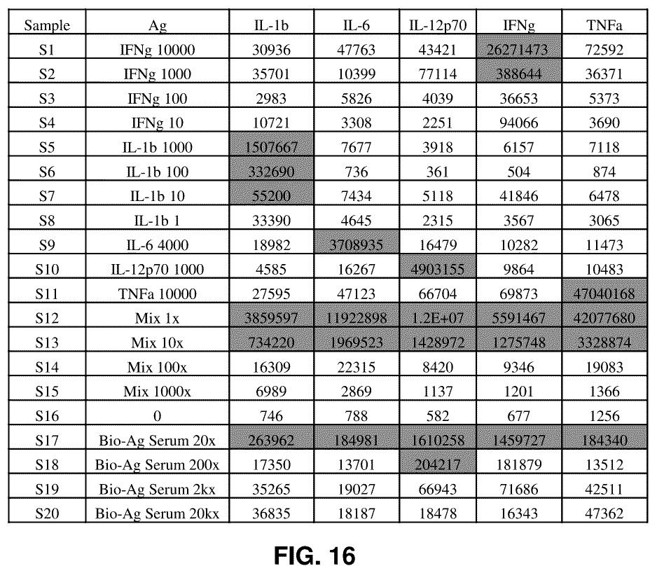

Although the background signal in this first test was relatively high, the results clearly demonstrated that all five cytokines were correctly detected. Sequence read counts correlated with cytokine concentrations, and multiplex detection showed comparable performance to singleplex detection (FIG. 16). In FIG. 16, the values significantly above the baseline for the same targets were highlighted in green, indicating the detection of these targets in the samples. For example, when the baseline of IL-1b is about 37000, cell values above 37000 for IL-1b were highlighted. Furthermore, all five cytokines were detectable in 20× diluted biotinylated serum samples, with IL-12p70 and IFN-γ remaining detectable even at 200× dilution.

Together, these findings demonstrated that NGS can be successfully integrated into the BIQ/BIQL platforms to achieve ultra-sensitive, specific, and multiplexed cytokine quantification. The system reliably distinguished individual targets through unique barcodes, maintained consistent performance between singleplex and multiplex assays, and allowed robust detection even in diluted biotinylated serum samples. These results established the feasibility of applying NGS-based readout in BIQ/BIQL systems, providing a scalable and high-throughput strategy for protein biomarker analysis.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

We claim:

1. A kit for detection of a target analyte in a sample, the kit comprising:
(a) a first reagent; and
(b) a second reagent,
wherein:
(i) the first reagent comprises magnetic particles modified with a first binding agent specific for the analyte and
(ii) the second reagent comprises a second binding agent specific for the analyte, conjugated to a nucleic acid barcode.

2. The kit of claim 1, wherein the kit is for detection of two or more target analytes in the sample, and wherein
(a) the modified magnetic particles comprise two or more first binding agents; and
(b) the second reagent comprises two or more second binding agent, each conjugated to a nucleic acid barcode,
wherein the two or more first binding agents and the two or more second binding agents form two or more binding sets,
wherein each binding set comprises one first binding agent and one second binding agent,
wherein the first binding agent and the second binding agent in each binding set are specific for the same target analyte, and
wherein the two or more analytes are different from each other.

3. The kit of claim 1, wherein the first binding agent and the second binding agent are independently an antibody, an aptamer, or a peptide, or a combination thereof.

4. The kit of claim 1, wherein the first binding agent is bound to the modified magnetic particle via an affinity binding pair selected form a group consisting of avidin-family protein/biotin, aptamer/target molecule pairs, receptor/ligand pairs, natural or synthetic receptor/ligand pairs, or amines and carbonyl compounds.

5. The kit of claim 4, comprising DAB/CAB pairs selected from Table 2, optionally herein the nucleic acid barcode is selected from the group consisting of SEQ ID NO:1-5 and SEQ ID NO: 20-24.

6. The kit of claim 1, wherein the nucleic acid barcode comprises:
(a) a region for hybridization with a TaqMan probe;
(b) a region for primer binding during PCR amplification; and
(c) a region for target specificity.

7. A kit for detection of one or more target analyte(s) in a sample, the kit comprising:
(a) a first reagent; and
(b) a second reagent,
wherein:
(i) the first reagent comprises magnetic particles modified with a member of an affinity binding pair, and
(ii) the second reagent comprises a second binding agent specific to the analyte and a nucleic acid barcode, wherein the second binding agent is conjugated to the nucleic acid barcode.

8. The kit of claim 7, wherein the kit is for detection of two or more target analytes in the sample, and wherein
(a) the magnetic particles are modified with a member of an affinity binding pair; and
(b) the second reagent comprises two or more second binding agents each specific to a target analyte, wherein each second binding agent is conjugated to a nucleic acid barcode,
wherein the two or more target analytes are different from each other.

9. The kit of claim 7, further comprising a protein labeling reagent.

10. The kit of claim 7, wherein the second binding agent is an antibody, an aptamer, or a peptide, or a combination thereof.

11. The kit of claim 7, wherein the member of the affinity binding pair is an avidin-family protein.

12. The kit of claim 9, wherein the protein labeling reagent is a biotinylation reagent.

13. The kit of claim 7, wherein the nucleic acid barcode is selected from the group consisting of SEQ ID NO: 1-5, SEQ ID NO:34, and SEQ ID NO:35.

14. A method of detecting one or more analytes in a sample using the kit of claim 1, the method comprising:
(i) incubating the sample with the modified magnetic particles and the second binding agent to form an immune complex; and
(ii) performing signal amplification and/or sequencing of the nucleic acid barcode, optionally wherein step (ii) is performed using qPCR or sequencing, or a combination thereof.

15. The method of claim 14, further comprising, after step (i),
(a) purifying the immune complex; and/or
(b) eluting the nucleic acid barcode from the immune complex using an elution buffer.

16. The method of claim 15, wherein step (a) comprises washing the immune complex with a wash buffer to remove unbound materials and separating the immune complex from the buffer using a magnetic field or centrifugation.

17. A method of detecting one or more analytes in a sample using the kit of claim 7, the method comprising:
(i) labeling proteins in the sample with the protein labeling reagent;
(ii) incubating the sample with the modified magnetic particles and the second reagent to form an immune complex; and
(iii) performing signal amplification and/or sequencing of the nucleic acid barcode.

18. The method of claim 17, wherein the protein labeling reagent is a biotinylation reagent.

19. The method of claim 17, further comprising, after step (ii),
(a) purifying the immune complex; and/or
(b) eluting the nucleic acid barcode from the immune complex using an elution buffer.

20. The method of claim 19, wherein step (a) comprises washing the immune complex with a wash buffer to remove unbound materials and separating the immune complex from the buffer using a magnetic field or centrifugation.
